(19) United States
(12) Patent Application Publication
(10) Pub. No.: US 2025/0060387 A1
(43) Pub. Date: Feb. 20, 2025

(54) A DEVICE FOR AN AUTOMATED DETERMINATION OF AN ANALYTE IN LIQUID PHASE AND A METHOD FOR AN AUTOMATED DETERMINATION OF AN ANAL YTE IN LIQUID PHASE USING THE DEVICE, IN PARTICULAR FOR MONITORING A PROGRESS OF A DIALYSIS

(71) Applicant: MICROANALYSIS Sp. z o.o., WARSZAWA (PL)

(72) Inventors: Lukasz TYMECK, WARSZAWA (PL); Michal MICHALEC, WARSZAWA (PL); Mateusz GRANICA, WARSZAWA (PL); Olga KOPACKA, WARSZAWA (PL); Lukasz JANUS, WARSZAWA (PL); Marcin GRZECZKOWICZ, WARSZAWA (PL); Agnieszka WIECKOWSKA, WARSZAWA (PL); Justyna BZURA, WARSZAWA (PL); Mateusz STAWICKI, WARSZAWA (PL)

(73) Assignee: MICROANALYSIS Sp. z o.o., WARSZAWA (PL)

(21) Appl. No.: 18/275,011

(22) PCT Filed: Jul. 12, 2023
(86) PCT No.: PCT/PL2023/000037

§ 371 Date: Jul. 30, 2023

(30) Foreign Application Priority Data
Jul. 12, 2022 (PL) P.441721
Jul. 11, 2023 (PL) P.445550
Jul. 11, 2023 (PL) P.445551
Jul. 11, 2023 (PL) P.445552

(51) Int. Cl. G01N 35/10; G01N 33/49; G01N 33/62; G01N 33/70; G01N 35/00; G01N 35/04

(52) U.S. Cl. CPC G01N 35/1016; G01N 33/491; G01N 33/4915; G01N 33/62; G01N 33/70; G01N 35/00732; G01N 35/1095; G01N 2035/009; G01N 2035/0434

(57) ABSTRACT

The invention relates to a device for the automatic determination of an analyte in the liquid phase by conducting a specific chemical reaction and subsequent optical measurement of the concentration of its products, using spectroscopic techniques, operating in stationary or flow mode. This device is equipped with a reaction-detection system with a replaceable cartridge. The device enables conducting chemical reactions in the liquid phase with the mixing of many streams of reagents, and also ensures the quantitative nature of the determination.

The invention also relates to a method for the automatic determination of an analyte in the liquid phase using the device according to the invention, in particular for monitoring the progress of the dialysis process.

The subject of the invention is a device is a device for the automated determination of an analyte in the liquid phase by conducting a specific chemical reaction and subsequent determination of its product using spectroscopic techniques, operating in stationary or flow mode. This device is equipped with a reaction-detection system with a replaceable cartridge. The device enables conducting chemical reactions in the liquid phase with the mixing of many streams of reagents, and also ensures the quantitative nature of the determination.

The progress in the automation of analytical processes and the miniaturisation of the equipment used has been observed over the last decades. There are known automated methods of conducting analytical and clinical tests, working in a flow mode, with a system controlled by pumps and valves, equipped with a sample changer. This allows to speed up the determination process, as well as to increase its precision and to eliminate human errors. The so-called lab-in-syringe method is known. This is an approach aimed at the mechanisation of the analytical procedure by collecting individual components of the reaction mixture into the syringe, as well as conducting a chemical reaction and determinations inside the syringe. Devices of this type work in conjunction with a system of valves directing the streams of solutions. Syringes are also used as pumps, but they do not constitute a reaction or detection space [Molecules 25 (2020) 1612; Molecules 26 (2021) 5358].

Parallel to the technological and apparatus progress, the progress of analytical techniques and their adaptation to flow systems is also observed. It is known to use ion-selective electrodes grouped into analytical blocks for flow determination of blood components [Analytical Chemistry 76 (2004) 6410-6418; Clinica Chimica Acta 411 (2010) 309-317]. It is also known to use screen-printed electrodes (e.g. reference electrodes, pH-metric electrodes, chloride electrodes) as detectors of analytes in physiological fluids in clinical studies carried out in flow conditions [Analytica Chimica Acta 526 (2004) 3-11]. Universal optical detectors are known, operating in the photometric and fluorometric regimes simultaneously, having a system of three optoelectronic elements, preferably LEDs, one of which acts as a light source, and the other two act as detectors [Talanta 198 (2019) 169-178].

The solutions mentioned above are used in many fields of modern technology, especially in analytical laboratories and medical diagnostic devices.

Haemodialysis progress tracking systems are known, aimed at determining the current blood purification status based on the analysis of the temporary concentration of uremic toxins in the post-dialysis fluid (e.g. urea, creatinine, phosphate ions, vitamin B12), which allows to determine the appropriate moment of the end of dialysis for a particular patient. This is extremely important because in the classical dialysis regime in medical centres, where patients receive a standard (non-personalised) dose of dialysis treatment and at standard length of the dialysis process, the effectiveness of dialysis (the degree of purification of the patient's blood) is determined post factum on the basis of the patient's blood composition collected after the dialysis treatment. This approach does not allow dialysis progress to be monitored in real time. Thus, it is not possible to prolong the dialysis treatment of the patients with high initial toxin levels (which would increase dialysis efficiency and patients' well-being), nor to terminate the dialysis treatment earlier for the patients with low initial toxin levels (which would increase patient comfort and save the operating time of haemodialysis machines).

A system for monitoring the level of urea in post-dialysis fluid based on the potentiometric determination of the concentration of ammonium ions is known (U.S. Pat. No. 5,442,969). Unfortunately, this system requires the use of a complicated pump system, as well as conducting of intermediate reactions to generate ammonium ions, which extends the measurement time and significantly reduces its accuracy.

There is known a device containing a multi-analyte enzymatic sensor for simultaneous monitoring of the concentration of many components of the post-dialysis fluid in real time, where the stream of this fluid is directed to a detection element equipped with in carriers containing enzymatic reagents deposited on them, reacting with the monitored components of the dialysate, and the products of these reactions are tracked using a detector, for example a colorimetric detector (WO 03097121 A2). Unfortunately, this device is burdensome to use due to the use of enzymatic reactions that require long-term determination, as well as ensuring proper measurement conditions (e.g. pH, temperature), proper carriers (e.g. membrane, paper) and proper preparation of enzyme reagents requiring storage at low temperature and their thermal pre-incubation before the measurement. Moreover, enzymatic sensors, due to the characteristics of enzymes deposited on the membrane, lose their activity during the measurement, which results in a systematic decrease in their sensitivity and requires frequent periodic exchange of the enzyme carriers.

There is known a microprocessor-controlled device for the biomedical analysis of body fluid, having a flow system to which the selected reagents are dosed from special containers in the form of bags, and the detection of the analyte is then carried out in real time (U.S. Pat. No. 5,258,314 A). Unfortunately, the use of this system is burdensome due to the method of dosing the reagents by squeezing them out of the bags by means of pressure applied by a set of squeezing rollers. The dosing system does not allow for smooth replenishment of the reagents, because each time the bags are refilled, the rollers must return to their starting position, which prevents continuous operation of the system. In addition, the device requires a one-time calibration at the beginning of the monitoring of a given patient, which requires strict temperature stabilisation throughout the entire measurement and significantly reduces its reliability.

There is known a device for monitoring the analyte level in the dialysate in real time, operating in the flow mode, which uses the classical reactions of inorganic reagents with uremic toxins (e.g. urea, creatinine, urease, phosphate ions) and colorimetric detection (PL 237447 B1). This device has a reservoir filled with a portion of pure dialysis fluid at the beginning of the treatment, which allows for additional calibration at each stage of the dialysis process, thanks to which the reliability of determinations increases, and the device does not require thermal stabilisation. The measurement process is greatly simplified thanks to the use of inorganic reagents that do not require special storage conditions, do not undergo aging and deactivation, do not require pre-incubation, and the reaction takes place quickly and without the use of additional functional elements, such as membranes. The system uses cartridges adapted to the determination of a specific analyte, equipped with reservoirs for appropriate chemical reagents and a diode optical detection system with a wavelength adapted to the determination of a specific analyte. The flow of the dialysate stream and the dosing of chemical reagents is carried out by means of a system of micropumps. Unfortunately, the low precision of the pumps significantly reduces the precision of determinations, and the system itself is characterised by low functional flexibility resulting from the need to change the cartridge when changing the analyte.

Automating the real-time liquid-phase analyte level monitoring process, especially in medical applications where samples are in the flow mode, requires careful protection of the source of the analytical material from contamination by the automated device for determination of the analyte during the sampling process. This is particularly important because the samples for testing are taken from a liquid stream with a system sensitive to microbial contamination upstream, such as a haemodialysis machine, and the well-being of dialysis patients requires that the haemodialysis machine is kept sterile. There is a risk of contaminants being transferred upstream along the walls of the vessels and hoses used in the system, especially when the analytical device is not disinfected between the serial dialysate determinations of subsequent patients.

Therefore, there is an unsatisfied need to develop a universal device for the automated determination of an analyte in the liquid phase by conducting specific chemical reactions and subsequent detection of their products, with high functional flexibility, operating in a stationary or flow regime, allowing for precision and accuracy of the measurements required by modern standards for analytical determinations and medical determinations, in particular for monitoring the progress of dialysis by tracking the changes in the composition of the dialysate, which will be characterised by simplicity of production and assembly, operational reliability, as well as simplicity and friendliness of use, while maintaining the sterility of the connection of this device with the sample source, especially in medical applications.

### SUMMARY

A device for the automated determination of an analyte in the liquid phase by conducting a specific chemical reaction and subsequent optical measurement of the concentration of its products, containing a hydraulic system equipped with a set of hoses for pumping liquid solutions and reactants, a system for sampling the tested solution, a reservoir for a standard solution, reservoirs for selected chemical reagents, and an optical detection system for the determination of the product of the specific reaction, where the reservoirs for liquids are placed in a replaceable cartridge, which device has a reaction space to mix the sampled portion of the tested solution or the reference solution or the standard solution with selected chemical reagents, as well as a space for optical detection of the products of the specific reaction, and the device is electronically controlled by the main controller, equipped with means for communication and information transfer with external electronic devices, characterised in that it has a reaction-detection system, where the reaction space takes the form of a cylinder (1) with pistons (2) moved by stepper motors (3), utilised to determine the selected analyte by conducting a specific chemical reaction and subsequent determination of its product using spectroscopic techniques, which system is equipped with a detection space (6) for quantitative determination of the product of the specific reaction in the post-reaction mixture, as well as equipped with an automated hydraulic system, allowing for precise sampling of the tested solution from the sample source (50,60,62), quantitative dosing of the tested solution and other reagents into the reaction space (1) and the detection space (6), as well as efficient gravitational pumping of the used liquid substances to the waste channel (61), whereby the flow of liquid in the hydraulic system is carried out pneumatically by changing the relative position of the pistons (2), and equipped with a replaceable cartridge (30/90) with reservoirs (20/95) to store reagents and standards, an optical detection system (70), an airlock (50) protecting the sample source against microbial contamination, and an alarm system (80) for verbalising messages regarding the determination process, equipped with a speaker (81), a light source (82) and means for remote communication (83).

According to the invention, the cylinder (1), with two pistons (2) moved by means of stepper motors (3) connected to them via the connectors (4), constitutes the reaction space in the housing block (5), which cylinder (1) has holes (10, 12,14,16) connected with channels (11,13,15,17) in the housing block (5) respectively, wherein these holes and channels have in pairs (10-11, 12-13, 14-15, 16-17) the same diameter, and each of the at least four openings (10) and corresponding channels (11) is detachably connected to one reservoir (20) in the cartridge (30) via ports (28) with stepped undercuts (29) equipped with side sealing gaskets (27) and a pressing lid (26), which detachably receive through pins (25) of the cartridge (30), connected by channels (24) to the sockets (23) detachably receiving the dispensing tips (22) of the reservoirs (20), where the hole (12) and the channel (13) equipped with a quick connect fitting (40) are detachably connected by a sampling hose (41) to the source of the sample, i.e. to the automatic sampling system (62), the pipe (60) with the sample stream or the airlock (50) on the pipe (60), while the hole (16) and the channel (17) equipped with a quick connect fitting (43) are detachably connected by a waste hose (44) to the waste channel (61), while the hole (14) and the channel (15) are connected to the detection chamber (6) in the form of a transverse through opening in the detection block (7), sealed from the outside with transparent windows (8) cooperating with the elements of the optical detection system (70), while the detection chamber (6) through the channel (18) equipped with the quick connect fitting (45) is detachably connected by a waste hose (46) to the hose (44) or to the waste channel (61). In the housing block (5), taking the form of a cuboid 85-105 mm long, 25-40 mm wide and 25-80 mm high, preferably with dimensions of 94×28×27 mm in the version with a separate detection block (7) or with dimensions of 94×28×57 mm in the version with an integrated detection block (7), the cylinder (1) is a through horizontal cavity, preferably with a circular cross-section, with an internal diameter in the range of 3-8 mm, preferably 4-7 mm, most preferably 6 mm, and a length in the range of 85-105 mm, preferably 94 mm, while its pistons (2), with a compatible outer diameter in the range of 3.2-8.2 mm, preferably 4.2-7.2 mm, most preferably 6.1 mm, tightly placed inside the cylinder (1), have piston rods made of a chemically inert, rigid plastic material such as polyethylene terephthalate (PTFE), polyetheretherketone (PEEK), poly(acrylonitrile-co-butadiene-co-styrene) (ABS), polyamide (PA) or polypropylene (PP), optionally in configuration with a gasket, preferably flat, and the guide holders of the piston rods made of metal such as brass, aluminium or steel, while the detection block (7) is in the form of a cuboid 30-50 mm long, 25-40 mm wide and 25-40 mm high, preferably with dimensions of 28×28×30 mm, or the detection block (7) is in the form of a cylinder with a diameter of 20-50 mm and a height of 25-40 mm, preferably 28 mm in diameter and 30 mm high, wherein the detection block (7) has at least one through opening constituting a detection chamber (6), preferably with a circular cross-section, with an internal diameter in the range of 3-10 mm, preferably 4-6 mm, most preferably 4 mm, sealed with transparent windows (8) made of a chemically inert material transparent in the range of determination of the product of specific reaction, preferably made of acrylic glass (PMMA), polycarbonate (PC) or polystyrene (PS), as well as a through channel (15) connecting the cylinder (1) with the detection chamber (6) and a through channel (18) connecting the detection chamber (6) with the quick connect fitting (45), wherein the channels (15,18) have a diameter in the range of 0.8-2 mm, preferably 1 mm, and preferably are perpendicular to the axis of the detection chamber (6), wherein the housing block (5) and the detection block (7) are made of a chemically inert, rigid material, preferably polyetheretherketone (PEEK), acrylic glass (PMMA), polyamide (PA) poly(acrylonitrile-co-butadiene-co-styrene) (ABS), aluminium or stainless steel, and preferably detection block (7) is rigidly and detachably connected to the housing block (5), and the detection chamber (6) is formed by two perpendicular, through openings, preferably perpendicular to the channels (15,18).

Alternatively, the cylinder (1), with two pistons (2) moved by means of stepper motors (3) connected to them via the connectors (4), is preferably horizontally oriented and tightly embedded inside the housing block (5) and tightly connected with it by its outer surface, is made of glass or quartz and equipped with at least one gasket (9), or is made of acrylic glass (PMMA), polystyrene (PS), polycarbonate (PC), poly(ethylene terephthalate) (PET) or polypropylene (PP), has holes (10, 12,14,16) connected with channels (11,13,15,17) in the housing block (5) respectively, wherein these holes and channels have in pairs (10-11, 12-13, 14-15, 16-17) the same diameter, and each of the at least four openings (10) and corresponding channels (11) is detachably connected to one reservoir (20) in the cartridge (30) via ports (28) with stepped undercuts (29) equipped with side sealing gaskets (27) and a pressing lid (26), which detachably receive through pins (25) of the cartridge (30), connected by channels (24) to the sockets (23) detachably receiving the dispensing tips (22) of the reservoirs (20), where the hole (12) and the channel (13) equipped with a quick connect fitting (40) are detachably connected by a sampling hose (41) to the source of the sample, i.e. to the automatic sampling system (62), the pipe (60) with the sample stream or the airlock (50) on the pipe (60), while the holes (14,16) and the channels (15,17) equipped with quick connect fittings (45,43) are detachably connected by a waste hose (46,44) to the waste channel (61), and furthermore, the housing block (5) has at least one a transverse through opening with a circular cross-section, constituting a detection chamber (6), revealing the transparent walls of the cylinder (1), allowing for the assembly of the elements of the optical detection system (70) on both its sides, while the detection chamber (6) and cylinder (1) are perpendicular to each other and their axes intersect, preferably directly above the outlet of the hole (14) and the channel (15). In the housing block (5) in the form of a cuboid 85-105 mm long, 25-40 mm wide and 25-80 mm high, preferably with dimensions of 94×28×33 mm, there is a cylindrical through opening with an internal diameter in the range of 5-15 mm receiving cylinder (1), preferably 10 mm or 12 mm per cylinder (1) or cylinder (1) with a seal (9), respectively, and the cylinder (1) has a length in the range of 85-105 mm, preferably 94 mm, and an internal diameter in the range of 3-8 mm, preferably 4-7 mm, most preferably 6 mm, and an outer diameter in the range of 4-14 mm, preferably 10 mm, where the preferred wall thickness of the cylinder (1) is 2 mm, while its pistons (2) having a compatible outer diameter in the range of 3.0-8.2 mm, preferably 3.15-7.2 mm, most preferably 6.1 mm, tightly and firmly seated inside the cylinder (1), have piston rods made of a chemically inert, rigid plastic material such as polyethylene terephthalate (PTFE), polyetheretherketone (PEEK), poly(acrylonitrile-co-butadiene-co-styrene) (ABS), polyamide (PA) or polypropylene (PP), optionally in configuration with a gasket, preferably flat, and the guide holders of the piston rods made of metal such as brass, aluminium or steel, while the housing block (5) has at least one through opening constituting the detection chamber (6), preferably with a circular cross-section, with an internal diameter in the range of 3-10 mm, preferably 4-6 mm, most preferably 4 mm, where the through channel (15) connects the cylinder (1) with the quick coupler (45), wherein the channel (15) has a diameter in the range of 0.8-2 mm, preferably 1 mm, while the housing block (5) is made of a rigid chemically inert material, preferably polyetheretherketone (PEEK), acrylic (PMMA), polyamide (PA), poly(acrylonitrile-co-butadiene-co-styrene) (ABS), aluminium or stainless steel.

Preferably, the inverse cartridge (30) has at least four reservoirs (20A,20B,20C,20D), preferably in the form of syringes with pistons (21), made of chemically inert materials, with a volume in the range of 5-12 ml, preferably 10 ml, with dispensing tips (22), preferably LUER, with outlets oriented downwards are embedded detachably in the sockets (23), preferably LUER or LUER LOCK, at the bottom of the housing (31) of the cartridge (30), wherein the cartridge (30) has a form of a container consisting of consists of at least a housing (31), a cover (32) and a lock (33), preferably a one-time lock, where the housing (31,32) of the cartridge (30) additionally has side sockets (34) for the forks (35) of the lift (36), made of one bent metal element fixed in four points on the lift (36), wherein the construction material of the cartridge (30) is thermoplastic, and additionally the cartridge (30) has an electronic system (39) equipped with a non-volatile memory (NFC RFID chip), wirelessly connected to the antenna of the electronic main controller (88) of the device when cartridge (30) docked in the device, which memory is recognised by the electronic main controller (88) to permit a single use of the cartridge (30).

Alternatively, the reaction space and the optical detection space is a cylinder (1) with transparent walls the range of determination of the product of the specific reaction, equipped with two opposing coaxial pistons (2) tightly sealing in on each side, moved by electronically controlled stepper motors (3), driving the pistons (2) in linear movement inside the cylinder (1), which is equipped with: a set of at least four hoses (99A,99B,99C,99D) embedded in the holes (10) in the wall of the cylinder (1), supplying liquid substances from at least four reservoirs (95A,95B,95C,95D) directly to the interior of the cylinder (1), including the tested solution from the sample source (50,60,62), as well as a hose (44) embedded in the hole (16) in the wall of the cylinder (1), removing liquid substances to the waste channel (61) directly from the interior of the cylinder (1), and at least one hose (47) embedded in the hole (14) in the wall of the cylinder (1), used to transfer gas and equalize the pressure inside the cylinder (1), while the fluid flow in the hydraulic system is carried out pneumatically by changing the relative mutual position of the pistons (2) generating gas pressure changes in a specific part of this system, forcing fluid movement to balance these changes, as well as at least one optical detection system (70), which components are placed around the cylinder (1) so that the optical path (72) connecting the light source (71) and the detector (74) passes through the interior of the cylinder (1).

Preferably, the normal cartridge (90) has at least four reservoirs (95A,95B,95C,95D), preferably in the form of conical vials, with a volume of 10-50 ml, preferably 25 ml, 50-100 mm high, preferably 78 mm, with the reservoirs (95) having a closure (96) at the top, preferably in the form of a cap or cork, with a hole of a diameter of 4-20 mm, preferably 6-14 mm, preferably closed with septum, having a 1 mm hole for pressure equalisation, where the reservoirs (95) are rigidly placed with the opening upwards in the sockets (97) receiving the closure (96) from the bottom, where the internal diameter of the sockets (97) corresponds to the external diameter of the closures (96), while the sockets (97) are preferably equipped with rigid tips (98) stabilising the outlets of the hoses (99) connecting the reservoirs (95) with the holes (10) in the cylinder (1), wherein the cartridge (90) has a form of a container having a frame (91) and elements (92,97) positioning the reservoirs (95), where the frame (91) cooperates with the guides (93) and the lift (94) for docking the cartridge (90) in the device, preferably the cartridge (90) consists of two interconnecting parts, where the first includes frame (91), cylinder (1) with pistons (2), connectors (4), hoses (99), gas hose/hoses (47), sockets (97) and guides with a stabilising element accommodating the second part (92) containing the positioning elements of the reservoirs (95), wherein the parts (91,92) are detachably connected to each other in an unambiguous manner, ensuring the concentricity of the reservoirs (95) and the sockets (97), thanks to which placing the reservoirs (95) is carried out in a repetitive manner, whereby the hose (41) connecting the cylinder (1) with the sample source (50,60,62) is divided into two fragments, one of which (41A), tightly seated in the hole (12), is located in the cartridge (90) and terminated with a connector (41B), and the other (41D) starts with a connector (41C) compatible with the connector (41B) and continues in the body of the device connecting to the sample source (50,60,62), while the hose (44), connecting the cylinder (1) with the waste channel (61), is divided into two fragments, one of which (44A), tightly seated in the hole (16), is located in the cartridge (90) and ends with a connector (44B), and the second one (44D) starts with a connector (44C), compatible with the connector (44B), and continues in the body of the device connecting to the waste channel (61).

Preferably, the optical detection system (70) consists of a light source (71), for example in the form of a diode, a fluorescent lamp or a light bulb, oriented with the front towards the interior of the detection space, and optionally one detector (74) or two detectors (74,75), for example in the form of a diode, photodiode, photoresistor, photomultiplier tube, CCD array or CMOS array, one of which (74), for photometric or turbidimetric detection, facing the interior of the detection space, is located on the axis of the optical path (72) of the light source (71) on the opposite side of the detection space, while the other detector (75), for fluorimetric or nephelometric detection, oriented with the front towards the interior of the detection space, is located on the axis of the optical path (73) crossing at 90° with the optical path (72) of the light source (71), wherein the light source (71) and detectors (74,75) can be guided to a desired location via optical fibres, wherein the light source (71) emitting light of adjustable wavelength, preferably equipped with a monochromator, or white light with a continuous spectrum, or monochromatic light in the range of absorption or excitation of the product of the specific reaction, or monochromatic light of several wavelengths in the range of absorption or excitation of the products of the specific reactions, while the detectors (74,75) are adapted to a specific analyte and a specific light source (71), and in particular the radiation of the light source (71) and the detectors (74,75) are adapted to the determination of creatinine, urea and phosphate ions, while monitoring the progress of the toxin removal process during the dialysis.

Preferably, the sample source is a classic sampling system (62) in the form of an automatic sample changer, or the sample source is a pipe with sample stream (60), or the sample source is an airlock (50) through which the sample stream is passing through the pipe (60), preferably the sample is taken from the accumulation reservoir (52) of the airlock (50) or its waste channel, wherein the airlock (50) is an open system and preferably the walls of the sample stream pipe (60) are not in contact with the housing of the main reservoir (51) and during the monitoring of the progress of the dialysis process, the sample source is an airlock (50) mounted on the pipe (60) with the dialysate stream flowing directly from the dialyser.

A method of automated determination of an analyte in the liquid phase by conducting a specific chemical reaction and subsequent optical measurement of the concentration of its products, characterised in that it uses the device for automated determination of an analyte in the liquid phase with a reaction-detection unit equipped with a replaceable cartridge (30/90), in particular for monitoring the progress of the dialysis process, described above, selecting a specific chemical reaction matching to a specific analyte and the wavelength for determining the product of this specific reaction, after which the device is adapted to the selected determination by adjusting the optical detection system (70), and adjusting the content of the cartridge (30/90) filling its first reservoir (20A/95A) with a standard solution, and two subsequent reservoirs (20B/95B,20C/95C) with chemical reagents necessary to carry out the specific reaction, where the fourth reservoir (20D) in the inverse cartridge (30) acts as a mixer, and the fourth reservoir (95D) in the normal cartridge (90) is used to store the matrix solution, wherein and then the cartridge (30/90) is placed in the device, and then the solution to be determined is taken by sucking its portion into the cylinder (1) through the sampling hose (41) from the sample source (60,62,50) or the standard solution from the reservoir (20A) or the matrix solution from the sample source (60,62,50) or the reservoir (95D) and then portions of chemical reagents are sequentially sampled from the two reservoirs (20B/95B,20C/95C) of the cartridge (30/90), into the cylinder (1), after which the reaction solution is mixed, and then the reaction solution is transferred to the area of the optical detection system (70), ensuring the liquid level in this area allowing the optical path (72) of the light source (71) to pass through the solution to be determined, preferably the level completely covering the optical path (72), and after a certain time the concentration of the product of the specific reaction is optically determined using the optical detection system (70) by photometric, turbidimetric, fluorimetric, nephelometric measurement, or their combination, and then the post-reaction solution is pumped out to the waste channel (61), cylinder (1) is cleaned by washing it with a fresh portion of the tested solution drawn into the cylinder (1) through the sampling hose (41), which is then pumped out from the cylinder (1) into the waste channel (61), wherein fluid flow in the hydraulic system is generated pneumatically by changing the relative position of the pistons (2) in the cylinder (1), and in cases where it is necessary to move the reaction solution to the desired area of the cylinder (1), the pistons (2) are moved in the hydraulic and pneumatic neutral mode with the same speed, direction and sense inside the cylinder (1), wherein while monitoring the progress of dialysis, when the analyte concentration readings indicate that the toxins have reached the normative level, which would be observed for a healthy person, indicating the effective purification of the patient's blood, the alarm system (80) is automatically activated, informing about the possibility of termination of the dialysis, or when the toxin level behaves abnormally, the alarm system (80) is automatically activated, informing about the possible errors in the dialysis process, and in both cases, the alarm system (80) automatically activates a sound and a light signal on the device, and sends information to peripheral devices such as a display on the device or the operator's phone, and the measurement for the proper sample is preceded by calibration measurements using the standard solution and the matrix solution.

Preferably, using the device in the variant III, immediately after sequential uptake of the tested sample and reagents from the reservoirs (20B,20C) of the cartridge (30) into the cylinder (1), they are sequentially pumped into the mixer (20D), and after taking and pumping all the solutions to the fourth reservoir (20D), the obtained reaction solution is mixed by its pumping between the cylinder (1) and the reservoir (20D), where the volume of the tested sample equals 30-90 μl, the volume of the reagents used equals 50-250 μl, which gives a reaction mixture of a volume of 240-320 μl, and after mixing the reaction solution, its portion, preferably 240 μl, is pumped from the mixer (20D) to the cylinder (1), and then through the hole (14) and channel (15) to the detection chamber (6), where the optical measurement is carried out allowing for the quantitative determination of the analyte, and after the determination, the reaction solution is pumped out form the detection chamber (6) and the reservoir (20D) through the channel (18) and a channel (17), respectively, to the waste channel (61).

Alternatively, using the device in the variant II, immediately after sequential uptake of the tested sample and reagents from the reservoirs (20B,20C) of the cartridge (30) into the cylinder (1), they are sequentially pumped into the mixer (20D), and after taking and pumping all the solutions to the fourth reservoir (20D), the obtained reaction solution is mixed by its pumping between the cylinder (1) and the reservoir (20D), where the volume of the tested sample equals 30-90 μl, the volume of the reagents used equals 50-250 μl, which gives a reaction mixture of a volume of 240-320 μl, and after mixing the reaction solution, its portion, preferably 240 μl, is pumped from the mixer (20D) to the cylinder (1), and then moved between the pistons (2) to the area of the detection chamber (6) in the cylinder (1), where the optical measurement is carried out allowing for the quantitative determination of the analyte, and after the determination, the reaction solution is pumped out form the detection chamber (6) and the reservoir (20D) through the channel (15) and a channel (17), respectively, to the waste channel (61).

Alternatively, using the device in the variant I, after sequential uptake of the test sample and reagents from the reservoirs (95B,95C) of the cartridge (90) into the cylinder (1) while replenishing or reducing the gas content in the cylinder (1) between the pistons (2) using the hole (14) and the gas hose (47), the obtained reaction solution is mixed by passing gas taken from the hose (47) through its volume, wherein the volume of the reaction solution being preferably 428-1040 μl in the system with a horizontal cylinder (1) or 565-1040 μl in the system with a vertical cylinder (1), where the width of the optical path (72) equals 5 mm and the distance between the projections of the holes (10,12,14,16) on the cylinder (1) axis equals 5 mm, and after mixing the reaction solution, a portion of the reaction solution is moved between the pistons (2) to the detection area in the cylinder (1), where the analyte is determined, and after the determination, the reaction solution is pumped out from the cylinder (1) from in the detection area through the opening (16) and the hose (44) to the waste channel (61).

According to the invention, to track the progress of blood dialysis by examining the changes in the level of the toxins in the stream of the post-dialysis fluid flowing out form artificial kidney through its waste channel (60), it is used
a device having one optical detection system (70) containing a light source (71) emitting light of adjustable wavelength or white light of a continuous spectrum, and a universal detector (74), or
a device having at least 3 optical detection systems (70) containing various light sources (71) emitting monochromatic light of a wavelength of 500-550 nm, preferably 525 nm, 410-460 nm, preferably 415 nm, and 550-900 nm, preferably 625 nm, and various detectors (74) of a wavelength of 525 nm, 460 nm and 625 nm, respectively, and identical detectors (75) of a wavelength of 625 nm, or
a device having a optical detection system (70) comprising a combined light source (71) emitting monochromatic radiation of a wavelength of 500-550 nm, preferably 525 nm, 410-460 nm, preferably 415 nm, and 550-900 nm, preferably 625 nm, and a universal detector (74),
wherein, before monitoring the progress of dialysis, the analyte (toxin) for the determination is selected from: creatinine, urea and phosphate ions, and then a standard solution is placed in the reservoir (20A/95A), and chemical reagents to conduct the specific reaction are placed in the reservoirs (20B/95B,20C/95C), respectively:
   when determining creatinine: standard creatinine aqueous solution [CAS 60-27-5], picric acid aqueous solution [CAS 88-89-1], and aqueous solution of NaOH[CAS 1310-73-2],
   for the determination of urea: standard aqueous solution of urea [CAS 54-13-6], aqueous-ethanolic solution of 4-(dimethylamine)benzaldehyde [CAS 100-10-7] and hydrochloric acid [CAS 7647-01-0], and aqueous solution of HCl [CAS 7647-01-0],
   and for the determination of phosphate ions: a standard aqueous solution of sodium phosphate [CAS:7601-54-9], an aqueous solution containing ammonium orthomolybdate [CAS 236-031-3], potassium antimonyl tartrate [CAS 28300-74-5], sulfuric acid [CAS 76664-93-9] and an aqueous solution of ascorbic acid [CAS: 50-81-7],

and then the cartridge (30/90) is mounted in the device, wherein the matrix solution is the pure dialysis fluid, which before starting the actual dialysis is sampled from the waste stream of the artificial kidney (60) through the airlock (50), after which a preliminary calibration measurement is carried out, and then the post-dialysis fluid is sampled from the waste stream of the artificial kidney (60) through the airlock (50), at given intervals, e.g. every 5-15 minutes, and the temporary concentration of the analyte in the dialysate stream (60) is determined, preferably washing the cylinder (1) between the sequential samplings and determinations of the analyte with a portion of the dialysate stream of the current composition, and preferably by carrying out calibration measurements between successive samplings and determinations of the analyte, using the standard solution from the reservoir (20A), simultaneously tracking on an external electronic device the decrease in the toxin content in the dialysate in the function of time.

### BRIEF DESCRIPTION OF DRAWINGS

The present invention describing the device for automated analyte determination in the liquid phase, equipped with a reaction-detection system with a replaceable cartridge, and the method of automated determination of the analyte in the liquid phase with the use of this device, in particular for monitoring the progress of the dialysis process, is described in detail below with reference to the drawings, FIGS. 1-17 for variant I, FIGS. 18-36 for variant II, FIGS. 33, 37-55 for variant III, and FIGS. 55-61 for the airlock.

FIG. 1 shows a spatial projection of the functional elements of
A) the device in variant I with a horizontal cylinder (1) and four reservoirs (95), with pistons (2), stepper motors (3) with lead screws and nuts positioning the connectors (4), holes (10,12,14,16), hoses (99,41,47,44), tips (98), reservoirs (95), sample channel (60) and waste channel (61) in the form of a flow pipe, and normal cartridge (90) in the basic version,
B) the normal cartridge (90) in the basic version with guides (93) and a set of sockets (97) with tips (98) for reservoirs (95);

FIG. 2 shows a spatial projection of the functional elements of
A) the devices in variant I with a horizontal cylinder (1) and four reservoirs (95), with pistons (2), stepper motors (3) with lead screws and nuts positioning the connectors (4), holes (10,12,14,16), hoses (99,41,47,44), reservoirs (95), sample channel (60) and waste channel (61) in the form of a flow pipe of the tested solution, and a normal cartridge (90) in the preferred version with two-element (91,92) construction,
B) the normal cartridge (90) in a two-piece version (91, 92), where the part (92) comprises a set of sockets (98) for the reservoirs (95);

FIG. 3 shows a scheme of the device in variant I with the cylinder (1) in a horizontal orientation with visible pistons (2), stepper motors (3) with lead screws and nuts positioning the connectors (4), hoses (99,41,47,44), reservoirs (95), sample channel (60) and waste channel (61) in the form of a flow pipe of the tested solution, and normal cartridge (90);

FIG. 4 shows a diagram of the device variant I with a cylinder (1) in a vertical orientation with visible pistons (2), stepper motors (3) with lead screws and nuts positioning the connector (4), hoses (99,41,47,44), reservoirs (95), sample channel (60) and waste channel (61) in the form of a flow pipe of the tested solution, and normal cartridge (90);

FIG. 5 shows the view of the cylinder (1):
A) in variant I in a horizontal orientation with visible pistons (2), hoses (99) running to reservoirs (95), hose (41) running to the sample source (50,60,62), hose (47) for gas transfer and hose (44) for removing the liquid substances to the waste channel (61),
B) in variant I in a vertical orientation with visible pistons (2), hose (41) running to the sample source (50,60,62), hoses (99) running to reservoirs (95), hose (44) removing the liquid substances to the waste channel (61);

FIG. 6 shows a scheme of the way that the optical path (72) passes through the reaction solution located between the pistons (2)
A) in the cylinder (1) in a horizontal orientation, with the minimum possible amount of the solution completely covering the optical path (72) passing through the axis of the cylinder (1) in the transverse view to the axis of the cylinder (1),
B) and in the view along the cylinder axis (1),
C) and also in the cylinder (1) in a vertical orientation, with the minimum possible amount of this solution completely covering the optical path (72) passing through the axis of the cylinder (1) in the transverse view to the axis of the cylinder (1),
D) and in the view along the cylinder axis (1);

FIG. 7 shows an exemplary sequence of the movements of the pistons (2) in the cylinder (1) in the horizontal orientation (A) and in the vertical orientation (B), of the device in variant I, consisting of with subsequent manipulations:
exemplary starting position of the pistons (2),
movement of the pistons (2) to the area of the hose (41),
uptake of a portion of the test solution from the sample source (50,60,62),
movement the pistons to the area of the hose (99B),
uptake of a portion of the reagent from the reservoir (95B),
movement the pistons (2) to the area of the hose (47),
drawing gas from the hose (47) and mixing the solution between the pistons (2),
movement of the pistons to the hose area (99C),
removal of an excess air, raising the level of the reaction solution,
movement of pistons (2) to the area of hose (44) and optical detection system (70),
determination of the analyte using the optical detection system (70),
removal of the reaction solution into the waste channel (61);

FIG. 8 shows the view of the cylinder (1) in variant I in a horizontal orientation with visible hoses (99,41,47,44) and an optical detection system (70) consisting of a light source (71) emitting light of an adjustable wavelength, in the form of a set comprising a light bulb and a monochromator, and two detectors (74,75) in the form of diodes, one of which (74) is located on the axis of the optical path (72) of the light source (71), and the other (75) is located on an optical path axis (73) oriented at 90° to the optical path (72);

FIG. 9 shows the view of the cylinder (1) in a horizontal orientation with visible hoses (99,41,47,44) and an optical detection system (70) consisting of a light source (71) emitting white light with a continuous spectrum, and a detector (74) in the form of a CMOS matrix;

FIG. 10 shows the view of the cylinder (1) in a horizontal orientation with visible hoses (99,41,47,44) and three optical detection systems (70), consisting of a light source (71) emitting monochromatic light in the form of a diode, and two detectors (74,75) in the form of diodes, one of which (74) is located on the axis of the optical path (72) of the light source (71), and the other (75) is located on the axis of the optical path (73) oriented at 90° to the optical path axis (72);

FIG. 11 shows the view of the cylinder (1) in a horizontal orientation with visible hoses (99,41,47,44) and an optical detection system (70), consisting of three monochromatic diode light sources (71) and three diode detectors (74) located in one plane, with their optical axes (74) crossing at one point on the axis of the cylinder (1) with an optical path (73) of a single diode detector (75);

FIG. 12 shows an exemplary spectrophotometric spectrum of the reaction solution for the determination of creatinine by the Jaffe method together with the corresponding calibration curve obtained with a conventional spectrophotometer;

FIG. 13 shows the dynamics of changes in the concentration of creatinine in the dialysate during the blood dialysis procedure, determined on the basis of:
A) measurements with a conventional spectrophotometer
B) measurements according to the invention through the cylinder (1) in the detection system (70) with a 525 nm diode as the light source (71) and a 525 nm diode as the detector (74);

FIG. 14 shows an exemplary spectrophotometric spectrum of the reaction solution for the determination of urea by the photometric method using Ehrlich reagent, together with the corresponding calibration curve obtained with a conventional spectrophotometer;

FIG. 15 shows the dynamics of changes in the concentration of urea in the dialysate during the blood dialysis procedure, determined on the basis of:
A) measurements with a conventional spectrophotometer
B) measurements according to the invention through the cylinder (1) in the detection system (70) with a 415 nm diode as the light source (71) and a 460 nm diode as the detector (74);

FIG. 16 shows an exemplary spectrophotometric spectrum of the reaction solution for the determination of phosphate ions by the phosphomolybdenate method together with the corresponding calibration curve obtained with a conventional spectrophotometer;

FIG. 17 shows the dynamics of changes in the concentration of phosphate ions in the dialysate during the blood dialysis procedure, determined on the basis of:
A) measurements with a conventional spectrophotometer
B) measurements according to the invention through the cylinder (1) in the detection system (70) with a 625 nm diode as the light source (71) and a 625 nm diode as the detector (74);

FIG. 18 shows a spatial view of the functional elements of the device in variant II with the cylinder (1) equipped with pistons (2) moved by stepper motors (3) with lead screws and positioning nuts of the connector (4), where the cylinder (1) is placed in the housing block (5) and is detachably connected through the holes (10,12,14,16) directly to the reservoirs (20) in the cartridge (30), a sample source (50,60,62), and a waste channel (61), while the detection chamber (6) is perpendicular to the cylinder (1);

FIG. 19 A) shows a spatial view of the device in a housing with the cartridge (30),
B) shows a spatial view of the device in a housing with the cartridge (30), placed on a cart having an airlock (50) inside;

FIG. 20 shows a scheme of the device in variant II with a cylinder (1) with pistons (2) moved by stepper motors (3) with lead screws and the nuts positioning the connectors (4), wherein the cylinder (1) is glued into a dedicated cavity in the one-piece detection block (5), and is connected through holes (10,12,14,16) with reservoirs (20) in the cartridge (30), the sample source (50,60,62) and the waste channel (61), while the detection chamber (6) is a transverse opening in the housing block (5) in the central part of the cylinder (1);

FIG. 21 shows a scheme of the device in variant II with a cylinder (1) with pistons (2) moved by stepper motors (3) with lead screws and the nuts positioning the connectors (4), wherein the cylinder (1) with a gasket (9) is placed in a dedicated cavity in a two-element detection block (5), and is connected through holes (10,12,14,16) directly to the reservoirs (20) in the cartridge (30), the sample source (50,60,62) and a waste channel (61), while and the detection chamber (6) is a transverse opening in the housing block (5) in the central part of the cylinder (1);

FIG. 22 shows the cartridge (30) docked through the lid (26) in the ports (27) at the housing block (5) of the cylinder (1), immobilised by the positioning pillars (38) on the upper surface of the housing block (5), entering the through holes (37) in the cartridge (30), wherein the four reservoirs (20) in the form of syringes with pistons (21) and central dispensing tips (22) of the LUER type, mounted in sockets (23) are connected through the channels (24) to the through pins (25) extending beyond the bottom outer surface of the housing (31) of the cartridge (30), docked in the ports (27) at the outlet of the channels (11) in the housing block (5) of the cylinder (1)
A. in a spatial realistic view,
B. in a spatial view with transparent walls of the presented elements;

FIG. 23 shows an exploded view of cartridge (30) with a housing (31), a lid (32) and a lock (33), equipped with four reservoirs (20) in the form of syringes with pistons (21) and central dispensing tips (22) of the LUER type, embedded in sockets (23) of the LUER type, connecting through channels (24) with through pins (25) extending beyond the bottom outer surface of the housing (31) of the cartridge (30):
A) in a spatial realistic view,
B) in a spatial view in vertical cross-section of the presented elements of the housing (31,32) of the cartridge (30);

FIG. 24 shows the housing block (5) of the cylinder (1) with pistons (2) and a lid (26), equipped with ports (27) with stepped undercuts (29) and gaskets (28) at the outlets of channels (11) of holes (10), with a transverse detection chamber (6) in the central part of the cylinder (1), glued into a dedicated cavity in the detection block (5):
A) in a front realistic view,
B) in a cross-section along a vertical plane passing through the axis of the cylinder (1),
C) in a front view with transparent walls of the presented elements,
D) in a top realistic view;

FIG. 25 shows the housing block (5) of the cylinder (1) with pistons (2) and a lid (26), equipped with ports (27) with stepped undercuts (29) and gaskets (28) at the outlets of channels (11) of holes (10), with a transverse detection chamber (6) in the central part of the cylinder (1), placed in a dedicated cavity in the detection block (5) with a gasket (9):
A) in a front realistic view,
B) in a cross-section along a vertical plane passing through the axis of the cylinder (1),
C) in a front view with transparent walls of the presented elements,
D) in a top realistic view;

FIG. 26 shows a view of a fragment of the cuboid housing block (5) with a detection chamber (6) in a form of a perpendicular circular opening perpendicular to the cylinder (1) and the channel (15), which axes are crossing with the axis of the detection chamber (6), equipped with an optical detection system (70) consisting of a light source (71) emitting light of adjustable wavelength, in the form of a light bulb and a monochromator, as well as a detector (74) in the form of a CCD matrix located on the axis of the optical path (72) of the light source (71);

FIG. 27 shows a view of a fragment of the cuboid housing block (5) with a detection chamber (6) in a form of a perpendicular circular opening perpendicular to the cylinder (1) and the channel (15), which axes are crossing with the axis of the detection chamber (6), equipped with an optical detection system (70) consisting of a light source (71) emitting white light of a continuous spectrum, and a detector (74) in the form of a CMOS matrix located on the optical path axis (72) of the light source (71);

FIG. 28 shows a view of a fragment of the cuboid housing block (5) with a detection chamber (6) in a form of a perpendicular circular opening perpendicular to the cylinder (1) and the channel (15), which axes are crossing with the axis of the detection chamber (6), equipped with an optical detection system (70) consisting of a light source (71) emitting monochromatic light, in the form of a diode, and a detector (74) in the form of a diode, located on the axis of the optical path (72) of the light source (71),

FIG. 29 shows a view of a fragment of the cuboid housing block (5) with a detection chamber (6) in a form of a perpendicular circular opening perpendicular to the cylinder (1) and the channel (15), which axes are crossing with the axis of the detection chamber (6), equipped with an optical detection system (70) consisting of a light source (71) emitting monochromatic light of several wavelengths, in the form of a integrated SMD diode, and a detectors (74) in the form of a CCD matrix with RGB filter, located on the optical path axis (72) of the light source (71;

FIG. 30 shows a scheme of the way the optical path (72) passes through the detection chamber (6) as an opening in the housing block (5), perpendicular to the cylinder (1) and the channel (15):
A) a side view along the axis of the optical path (72) of the light source (71),
B) in the view along the axis of the cylinder (1);

FIG. 31 shows a scheme of the way the optical path (72) passes through the detection chamber (6) in the form of two intersecting perpendicular circular openings in the housing block (5), perpendicular to the cylinder (1), oriented at an angle of 45° to the channel (15):
A) in a transverse view to the axis of the cylinder (1),
B) in the view along the axis of the cylinder (1);

FIG. 32 shows an exemplary sequence of movement of the pistons (2) in the cylinder (1) during a routine determination of an analyte, consisting of successive manipulations, including:
I. uptake of the sample:
   movement of the pistons (2) from the starting position to the area of the hole (12),
   uptake of a portion of the tested solution from the sample source (50,60,62),
   movement of the pistons (2) to the area of the hole (10D),
   pushing out the entire solution from between the pistons (2) to the reservoir (20D),
II. uptake of the first reagent:
   movement the pistons (2) to the area of the hole (10B),
   uptake of a portion of the reagent from the reservoir (20B) to the cylinder (1),
   movement the pistons (2) to the area of the hole (10D),
   pushing out the entire solution from between the pistons (2) to the reservoir (20D),
III. uptake of the second reagent:
   movement the pistons (2) to the area of the hole (10C),
   uptake of a portion of the reagent from the reservoir (20C) to the cylinder (1),
   movement the pistons (2) to the area of the hole (10D),
   pushing out the entire solution from between the pistons (2) to the reservoir (20D),
IV. mixing of the reaction solution:
   uptake of the entire solution from the reservoir (20D) to the cylinder (1),
   pushing out the entire solution from between the pistons (2) to the reservoir (20D),
   uptake of the entire solution from the reservoir (20D) to the cylinder (1),
   pushing out the entire solution from between the pistons (2) to the reservoir (20D),
V. optical detection:
   uptake of a portion of the solution from the reservoir (20D) to the cylinder (1),
   movement the pistons (2) to the area of the detection chamber (6),
   determination of the analyte using the optical detection system (70),
   pushing out the entire solution to the waste channel (61) through the hose (46),
VI. removal of the excess of the post-reaction solution:
   movement the pistons (2) to the area of the hole (10D),
   uptake of the entire solution from the reservoir (20D) to the cylinder (1),
   movement the pistons (2) to the area of the hole (16),
   pushing out the entire solution from between the pistons (2) to the waste channel (61),
VII. washing the reaction system (1,20D) with fresh sample:
   movement of the pistons (2) to the area of the hole (12),
   washing the cylinder (1) by uptake of the solution from the sample source (50,60,62),
   movement of the pistons (2) to the area of the hole (14),
   pushing out the solution through the hole (14),
   movement of the pistons (2) to the area of the hole (12),
   washing the cylinder (1) by uptake of the solution from the sample source (50,60,62),
   movement the pistons (2) to the area of the hole (10D),
   washing the reservoir (20D) by pushing out the solution to the reservoir (20D),
   uptake of the entire solution from the reservoir (20D) to the cylinder (1),
   movement the pistons (2) to the area of the hole (16),
   pushing out the entire solution to the waste channel (61) through the hose (44),
   movement the pistons (2) to the starting position,

FIG. 33 shows the functional elements of the lift (36) equipped with forks (35), compatible with sockets (34) of the cartridge (30), used for its docking in ports (28) in spatial views with a transparent cartridge (30) mounted on the forks (35):
A) side view,
B) front view,
C) spatial view;

FIG. 34 A) shows the graph of correlation of the results of creatinine determinations in the dialysate obtained using the device according to the invention (y-axis) with the results obtained in the hospital laboratory using Hitachi-Roche Cobas 6000 analyser (x-axis),
B) exemplary course of dialysis with monitoring of creatinine level in dialysate (device according to the invention) and in blood (Hitachi-Roche Cobas 6000 method);

FIG. 35 A) shows the graph of correlation of the results of urea determinations in the dialysate obtained using the device according to the invention (y-axis) with the results obtained in the hospital laboratory using Hitachi-Roche Cobas 6000 analyser (x-axis),
B) exemplary course of dialysis with monitoring of urea level in dialysate (device according to the invention) and in blood (Hitachi-Roche Cobas 6000 method);

FIG. 36 A) shows the graph of correlation of the results of phosphate ions determinations in the dialysate obtained using the device according to the invention (y-axis) with the results obtained in the hospital laboratory using Hitachi-Roche Cobas 6000 analyser (x-axis),
B) exemplary course of dialysis with monitoring of phosphate ions level in dialysate (device according to the invention) and in blood (Hitachi-Roche Cobas 6000 method);

FIG. 37 shows a spatial view of the functional elements of the device in a preferred variant III with a cylinder (1) equipped with pistons (2) moved by stepper motors (3) with lead screws and nuts positioning the connectors (4), where the cylinder (1) is a cavity in the housing block (5) and is detachably connected to the detection block (7), which cylinder (1) through the holes (10,12,14,16) is directly connected to the reservoirs (20) in the cartridge (30), a sample source (50,60,62), a detection chamber (6) and a waste channel (61);

FIG. 38 A) shows a spatial view of the device in a housing with the cartridge (30),
B) shows a spatial view of the device in a housing with the cartridge (30), placed on a cart having an airlock (50) inside;

FIG. 39 shows a scheme of the device in variant Ill with a cylinder (1) equipped with pistons (2) moved by stepper motors (3) with lead screws and nuts positioning the connectors (4), where the cylinder (1) is a cavity in the housing block (5) and is detachably connected to the detection block (7), which cylinder (1) through the holes (10,12,14,16) is directly connected to the reservoirs (20) in the cartridge (30), the sample source (50,60,62), detection chamber (6) and the waste channel (61);

FIG. 40 shows a diagram of the device in variant Ill with a cylinder (1) equipped with pistons (2) moved by stepper motors (3) with screws and nuts positioning the connector (4), where the cylinder (1) is a cavity in the housing block (5) and is permanently connected with the detection block (7), which cylinder (1) through the holes (10,12,14,16) is directly connected to the reservoirs (20) in the cartridge (30), the sample source (50,60,62), detection chamber (6) and the waste channel (61);

FIG. 41 shows the cartridge (30) docked through the lid (26) in the ports (27) at the housing block (5) of the cylinder (1), immobilised by the positioning pillars (38) on the upper surface of the housing block (5), entering the through holes (37) in the cartridge (30), wherein the four reservoirs (20) in the form of syringes with pistons (21) and central dispensing tips (22) of the LUER type, mounted in sockets (23) are connected through the channels (24) to the through pins (25) extending beyond the bottom outer surface of the housing (31) of the cartridge (30), docked in the ports (27) at the outlet of the channels (11) in the housing block (5) of the cylinder (1)
C. in a spatial realistic view,
D. in a spatial view with transparent walls of the presented elements;

FIG. 42 shows an exploded view of cartridge (30) with a housing (31), a lid (32) and a lock (33), equipped with four reservoirs (20) in the form of syringes with pistons (21) and central dispensing tips (22) of the LUER type, embedded in sockets (23) of the LUER type, connecting through channels (24) with through pins (25) extending beyond the bottom outer surface of the housing (31) of the cartridge (30):
C) in a spatial realistic view,
D) in a spatial view in vertical cross-section of the housing (31,32) of the cartridge (30);

FIG. 43 shows the housing block (5) of the cylinder (1) with the pistons (2) and the lid (26), equipped with ports (27) at the outlets of the channels (11) of the holes (10), detachably connected to the detection block (7) using a gasket (19):
A) in a front realistic view,
B) in a cross-section along a vertical plane passing through the axis of the cylinder (1),
C) in a front view with transparent walls of the presented elements,
D) in a top realistic view;

FIG. 44 shows the housing block (5) of the cylinder (1) with the pistons (2) and the lid (26), equipped with ports (27) with stepped undercuts (29) and seals (28) at the outlet of the channels (11) of the holes (10), integrated with the detection block (7):
A) in a front realistic view,
B) in a cross-section along a vertical plane passing through the axis of the cylinder (1),
C) in a front view with transparent walls of the presented elements,
D) in a top realistic view;

FIG. 45 shows a view of the cuboid detection block (7) with the detection chamber (6) in a form of two perpendicular circular openings blinded with transparent windows (8), perpendicular to the coaxial channels (15,18), equipped with an optical detection system (70) consisting of a light source (71) emitting light of adjustable wavelength, in the form of a light bulb and a monochromator, as well as two detectors (74,75) in the form of CCD matrices, one of which (74) is located on the optical path axis (72) of the light source (71) and the other (75) is located on the optical path axis (73) perpendicular to the optical path (72);

FIG. 46 shows a view of the cuboid detection block (7) with the detection chamber (6) in a form of two perpendicular circular openings blinded with transparent windows (8), perpendicular to the coaxial channels (15,18), equipped with an optical detection system (70) consisting of a light source (71) emitting white light of a continuous spectrum, and a detector (74) in the form of a CMOS matrix located on the optical path axis (72) of the light source (71);

FIG. 47 shows a view of the cuboid detection block (7) with the detection chamber (6) in a form of two perpendicular circular openings blinded with transparent windows (8), perpendicular to the coaxial channels (15,18), equipped with an optical detection system (70) consisting of a light source (71) emitting monochromatic light, in the form of a diode, and two detectors (74,75) in the form of diodes, one of which (74) is located on the axis of the optical path (72) of the light source (71) and the other (75) is located on the axis of the optical path (73) perpendicular to the optical path (74);

FIG. 48 shows a view of the cuboid detection block (7) with the detection chamber (6) in a form of two perpendicular circular openings blinded with transparent windows (8), perpendicular to the coaxial channels (15,18), equipped with an optical detection system (70) consisting of a light source (71) emitting monochromatic light of several wavelengths, in the form of a integrated SMD diode, and a detector (74) in the form of a CCD matrix with an RGB filter, located on the axis of the optical path (72) of the light source (71);

FIG. 49 shows a scheme of the way the optical path (72) passes through the detection chamber (6) in the form of a circular opening in the detection block (7), perpendicular to the coaxial channels (15,18):
A) a side view along the axis of the optical path (72) of the light source (71),
B) a top view along the axis of the channels (15,18);

FIG. 50 shows a scheme of the way the optical path (72) passes through the detection chamber (6) in the form of two intersecting perpendicular circular cavities in the detection block (7), perpendicular to the coaxial channels (15,18):
A) a side view along the axis of the optical path (72) of the light source (71),
B) a top view along the axis of the channels (15,18);

FIG. 51 shows an exemplary sequence of movement of the pistons (2) in the cylinder (1) during a routine determination of an analyte, consisting of successive manipulations, including:
I. uptake of the sample:
   movement of the pistons (2) from the starting position to the area of the hole (12),
   uptake of a portion of the tested solution from the sample source (50,60,62),
   movement of the pistons (2) to the area of the hole (10D),
   pushing out the entire solution from between the pistons (2) to the reservoir (20D),
II. uptake of the first reagent:
   movement the pistons (2) to the area of the hole (10B),
   uptake of a portion of the reagent from the reservoir (20B) to the cylinder (1),
   movement the pistons (2) to the area of the hole (10D),
   pushing out the entire solution from between the pistons (2) to the reservoir (20D),
III. uptake of the second reagent:
   movement the pistons (2) to the area of the hole (10C),
   uptake of a portion of the reagent from the reservoir (20C) to the cylinder (1),
   movement the pistons (2) to the area of the hole (10D),
   pushing out the entire solution from between the pistons (2) to the reservoir (20D),
IV. mixing of the reaction solution:
   uptake of the entire solution from the reservoir (20D) to the cylinder (1),
   pushing out the entire solution from between the pistons (2) to the reservoir (20D),
   uptake of the entire solution from the reservoir (20D) to the cylinder (1),
   pushing out the entire solution from between the pistons (2) to the reservoir (20D),
V. optical detection:
   uptake of a portion of the solution from the reservoir (20D) to the cylinder (1),
   movement the pistons (2) to the area of the hole (14),
   pushing out the entire solution from between the pistons (2) to the detection chamber (6),
   determination of the analyte using the optical detection system (70),
VI. removal of the excess of the post-reaction solution:
   movement the pistons (2) to the area of the hole (10D),
   uptake of the entire solution from the reservoir (20D) to the cylinder (1),
   movement the pistons (2) to the area of the hole (16),
   pushing out the entire solution from between the pistons (2) to the waste hose (44),
VII. washing the reaction system (1,20D) and detection chamber (6) with fresh sample:
   movement of the pistons (2) to the area of the hole (12),
   washing the cylinder (1) by uptake of the solution from the sample source (50,60,62),
   movement of the pistons (2) to the area of the hole (14),
   washing the detection chamber (6) by pushing out the solution through the hole (14),
   movement of the pistons (2) to the area of the hole (12),
   washing the cylinder (1) by uptake of the solution from the sample source (50,60,62),
   movement the pistons (2) to the area of the hole (10D),
   washing the reservoir (20D) by pushing out the solution to the reservoir (20D),
   uptake of the entire solution from the reservoir (20D) to the cylinder (1),
   movement the pistons (2) to the area of the hole (16),
   pushing out the entire solution to the waste channel (61) through the hose (44),
   movement the pistons (2) to the starting position,

FIG. 52 A) shows the graph of correlation of the results of creatinine determinations in the dialysate obtained using the device according to the invention (y-axis) with the results obtained in the hospital laboratory using Hitachi-Roche Cobas 6000 analyser (x-axis),
B) exemplary course of dialysis with monitoring of creatinine level in dialysate (device according to the invention) and in blood (Hitachi-Roche Cobas 6000 method);

FIG. 53 A) shows the graph of correlation of the results of urea determinations in the dialysate obtained using the device according to the invention (y-axis) with the results obtained in the hospital laboratory using Hitachi-Roche Cobas 6000 analyser (x-axis),
B) exemplary course of dialysis with monitoring of urea level in dialysate (device according to the invention) and in blood (Hitachi-Roche Cobas 6000 method);

FIG. 54 A) shows the graph of correlation of the results of phosphate ions determinations in the dialysate obtained using the device according to the invention (y-axis) with the results obtained in the hospital laboratory using Hitachi-Roche Cobas 6000 analyser (x-axis),
B) exemplary course of dialysis with monitoring of phosphate ions level in dialysate (device according to the invention) and in blood (Hitachi-Roche Cobas 6000 method);

FIG. 55 shows a scheme of the connection of the device to the sample source (60) through the airlock (50) located on the channel (60) upstream to the sampling point by a hose (41);

FIG. 56 shows a scheme of the airlock (50) and the way it is use to connect the device for the automated determination of an analyte in the liquid phase to the channel with the sample stream (60) via this airlock (50) located on the channel (60) above the sampling point by a hose (41) located:
A. on the waste channel of the main reservoir (51) below the valve (55),
B. on the waste channel of the accumulation reservoir (52) below the valve (54);

FIG. 57 shows a scheme of the preferred airlock (50) and the way it is use to connect the device for the automated determination of an analyte in the liquid phase to the channel with the sample stream (60) via this airlock (50) located on the channel (60), wherein the sampling aby the hose (41) takes place directly from the accumulation reservoir (52) upstream to the valve (54);

FIG. 58 shows a visualization of the airlock (50), in the preferred embodiment:
A. in a spatial realistic view,
B. in a spatial view with transparent walls of the main reservoir (51);

FIG. 59 shows a detailed view of the main reservoir (51) of the airlock (50), in the preferred embodiment:
A. in a spatial realistic view,
B. in a spatial view with transparent walls of the main reservoir (51);

FIG. 60 shows axonometric views of structural elements of the main reservoir (51) connected to its side walls, with functional drillings visible, in the preferred embodiment:
A. top lid of the main reservoir (51) in the outside view,
B. top lid of the main reservoir (51) in the inside view,
C. bottom lid of the main reservoir (51) in the outside view,
D. bottom lid of the main reservoir (51) in the inside view;

FIG. 61 shows a spatial view of the airlock (50) placed in a cart on which the device for the automated determination of an analyte in the liquid phase, according to the invention, is placed while monitoring the dialysis process,
A. rear view of the cart with back cover closed,
B. rear view of the cart with back cover open.

### DETAILED DESCRIPTION

The device according to the invention allows for automated determination of the analyte in liquid samples of any characteristics, both in the stationary and the flow regime. According to the invention, the device can be used in particular for monitoring changes in the concentration of uremic toxins in the dialysate during the blood dialysis process of the patients with renal failure using haemodialysis machines. The use of the device then allows for quick detection of dangerous situations resulting from complications of the dialysis process, as well as for determining the optimal end point of the dialysis, at the time of actual purification of the blood from toxins, which improves the well-being of the patients and allows for optimisation of the use of the operating time of haemodialysis machines. The present device is an improvement of analogous devices known from the state of the art and an improvement of the so-called lab-in-syringe method, considering the replacement of a number of components controlling the flow of fluids in the hydraulic system with one set of precise stepper motors with accessories, cooperating with a single cylinder (1) as a reaction space. Innovative use of a single cylinder (1), equipped with a pair of pistons (2) and a set of holes (10,12,14,16) enabling direct injection of fluids into its interior and pumping the fluids out from its interior allows the use of this cylinder (1) at the same time as the pump and the reaction space, as well as an efficient transfer of the postreaction solution of the detection space without the need of using further valves, pumps and other hydraulic components, which is a common inventive idea for all the variants of the current invention described below.

The device is equipped with a reaction-detection system with a replaceable cartridge (30/90) for storing the reagents and other liquids necessary during the determination. In addition, the device is equipped with an optical detection system (70), an airlock (50), an alarm system (80) and an electronic main controller (88) controlling the course of the determination process.

The invention, considering the reaction-detection system and construction of the cartridge can be implemented in the following embodiments.

The reaction-detection system is in the form of a cylinder (1) equipped with pistons (2), generating fluid flow in the hydraulic system of the device. The cylinder (1) can be both a reaction space (variants I, II, III) and a detection space (variants I and II). Then, the detection chamber (6) is the area of the cylinder (1) at its intersection with the optical detection system (70), which requires a cylinder (1) with a transparent side wall. Alternatively, the reaction space inside the cylinder (1) can be separated from the detection space (variant III). Then, the detection chamber (6) is located at the intersection of the channel (15,18) coming directly from the cylinder (1) with the optical detection system (70), which allows the use of the cylinder (1) with an opaque side wall, while providing the detection chamber (6) with transparent walls.

The replaceable cartridge has a structure conditioned by the type of reservoirs for liquids and reagents. The reservoirs (95) in the normal cartridge (90) have the outlet directed upwards, which is results in the need of exchanging the fluids with the cylinder (1) in the conditions of an open system, generating periodic need for gas exchange in the reaction-detection system during operation (variant I). Preferably, the reservoirs (20) in the inverse cartridge (30) have an outlet directed downwards, which ensures the possibility of exchanging the fluids with the cylinder (1), without the need for gas exchange in the reaction-detection system during operation (variants II and III).

The reaction-detection system in variant I with a replaceable normal cartridge (90) enables automated determination of the analyte in the liquid samples of any characteristics, both in the stationary and flow regime, and in particular this solution can be used in monitoring changes in the concentration of uremic toxins in the dialysate during the blood dialysis procedure of the patients with renal failure using haemodialysis machines. The cylinder (1) is made of a transparent material, which enables optical detection in the detection space which is a selected fragment of the reaction space inside it.

The reaction-detection system in variant II with a replaceable inverse cartridge (30) is characterised by increased mechanical strength of the structural elements and their greater resistance durability, and at the same time allows for a significant simplification of production and assembly, and ensures greater user-friendliness for the end users. At the same time, the reaction-detection system conducts all the technical and analytical functions of the system in variant I, and it is possible to use it in monitoring changes in the concentration of uremic toxins in the dialysate during the blood dialysis treatment of the patients with renal failure using haemodialysis machines. The housing block (5) of the cylinder (1) allows to increase the strength and tightness of the system. The use of the inverse cartridge (30) allows to simplify the topography of the hydraulic system, which allows for greater precision in dosing reagents for the reaction. In addition, the method of conducting the determinations has been improved, which now uses the fourth reservoir (20D) of the cartridge (30) as a mixer, which allows to increase the volume of the reagent portions used, and thus results in the increase in the precision of the determinations. The introduction of the housing block (5) as a key element ensuring mechanical protection and rigidity of the transparent cylinder (1) allows for production of the key elements of the hydraulic system with simple and well-known classical methods of machining the rigid materials, which reduces production costs and reduces the manufacturer's requirements.

The reaction-detection system in the preferred variant III, with a replaceable inverse cartridge (30), is characterised by increased mechanical strength of the structural elements and greater durability, and at the same time allows for a significant further simplification of production and assembly. At the same time, the reaction-detection system conducts all the technical and analytical functions of the system in variant I and II, and it is possible to use it in monitoring the changes in the concentration of uremic toxins in the dialysate during the blood dialysis treatment of the patients with renal failure using haemodialysis machines. The reaction space, realised in a non-transparent cylinder (1) in the housing block (5), is separated from the detection space, realised in the detection chamber (6) in the detection block (7). The introduction of the housing block (5) and the detection block (7) allows to increase the strength and tightness of the system, as well as to exclude the need of machining the cylinders (1) of transparent materials. The use of the inverse cartridge (30) allows to simplify the topography of the hydraulic system, which allows for greater precision in dosing the reagents for the reaction. Elimination of the transparent cylinder (1) and replacement of its role by a dedicated cavity in the housing block (5), as well as separation of the reaction area in the cylinder (1) from the detection area in the detection chamber (6) allows for production of the key elements of the hydraulic system with simple and well-known classical methods of machining the rigid materials, which reduces production costs and reduces the manufacturer's requirements.

The airlock (50), cooperating with each of the above variants of the device, allows to maintain the microbiological safety of the source of the analytical material that produces an uninterrupted stream of sample, such as a haemodialysis machine during the blood dialysis process, while ensuring reliability and simplicity of construction, and smooth sample flow. In addition, the sampling system in the form of a hose (41) located inside the accumulation reservoir (52) above the valve (54) allows to maximise the efficiency of the use of the accumulated portion of the sample, while ensuring the possibility of repeating the measurement of a given sample collected at a specific point in the measurement sequence.

### DETAILED DESCRIPTION OF THE INVENTION

The device for the automatic determination of an analyte in the liquid phase by conducting specific chemical reactions and subsequent optical measurement of the concentration of their products solves the nuisance known from the state of the art, consisting of low precision of determination, low functional flexibility as well as high price and high degree of complexity of known devices of this type.

The device according to the present invention provides high precision of determination thanks to the unique hydraulic system and unprecedented functional flexibility thanks to the use of the optical detection system (70) operating in various modes of optical determination. The simplicity of the design of the device ensures easy production, low price and the ability to adapt to specific analytical or medical applications. Thanks to the use of an airlock (50), it allows the use of the device in medical analyses while ensuring microbiological safety for the patients and medical equipment.

### Reaction-Detection System

The key element of the hydraulic system is the cylinder (1) with two coaxial opposing pistons (2) closing this cylinder (1) from both sides. The pistons (2) are moved by precise, standardised identical stepper motors (3) equipped with lead screws with the same pitch and characteristics, with the same positioning nuts. The invention considers trapezoidal lead screws, ball lead screws or other type lead screws, depending on the chosen solution, while the type of the positioning nuts is adapted to the type of lead screws used. Preferably, ball lead screws are used. It is possible to use different stepper motors (3) and different lead screws with different positioning screws, but this unnecessarily complicates the process of controlling the movement of the pistons (2) and pumping fluids in the hydraulic system. The positioning nuts are seated in a non-rotating connector (4) which is tightly connected to the pistons (2). The pistons (2) slide in a linear motion inside the cylinder (1) along its axis. The stepper motors (3) are electronically controlled by the electronic main controller (88), which controls the entire device, while the movement of the lead screws generates a linear movement of the pistons (2) inside the cylinder (1). In a preferred variant, the lead screws are arranged parallel or coaxially with the pistons (2), thanks to which the movement of the positioning nuts is the same as the movement of the pistons (2). The solution with parallel but misaligned pistons (2) and lead screws is particularly advantageous because it allows a significant reduction in the geometric dimensions of the entire device by avoiding the need of addition the length of the cylinder (1), the length of two pistons (2), the length of two stepper motors (3) with the length of two lead screws.

Reaction-Detection System with Cartage According to the First Embodiment (Variant I)

The cylinder (1) is also a pump that controls the flow of the fluids in the hydraulic system, as well as a space for conducting a specific reaction and a space for optical detection of the products of this reaction. The walls of the cylinder (1) are transparent in the range of optical determination of the product of the specific reaction, preferably, they are transparent in the range of visible light, near infrared and near ultraviolet, which ensures the possibility of conducting a variety of specific reactions. The cylinder (1) has an internal diameter in the range of 4-20 mm, preferably 8-16 mm, most preferably 12 mm. Pistons (2), one-piece or two-piece (i.e. equipped with a gasket), have an outer diameter matching to the size of the cylinder (1) ensuring tightness of the system, in the range of 4-20 mm, preferably 8-16 mm, most preferably 12 mm, i.e. the dimension of the pistons (2) is the same as the dimension of the cylinder (1). The cylinder (1) is made of glass, quartz, polypropylene (PP), polyethylene terephthalate (PET), acrylic (PMMA), polycarbonate (PC), polyamide (PA) or other transparent inert material, and the pistons (2) are preferably made of glass, quartz, polypropylene (PP), polyethylene terephthalate (PET), polyethylene terephthalate (PTFE), polyetheretherketone (PEEK), steel or other inert material, with or without the gasket. In a preferred variant, a body of a commercially available syringe devoid of the bottom with the needle seat is used as the cylinder (1), while the pistons of the same commercially available syringe are used as the pistons (2).

The present invention is an extension of the state-of-the-art idea of conducting determinations inside an ordinary syringe (so-called lab-in-syringe) [Molecules 25 (2020) 1612; Molecules 26 (2021) 5358]. Unlike earlier solutions, however, the present invention uses a modified syringe with two pistons (2) instead of a single piston, not only to force the flow of liquids in the hydraulic system, but also to fully control the stoichiometry of the reaction and determine its products without the need of using additional valves and other components to control the flow of the liquids. According to the present invention, in variant I, the cylinder (1) is directly connected to the reagent reservoirs (95) in the normal cartridge (90) through the holes (10) and the hoses (99) embedded therein. In turn, through the hole (12) and the hose (41), the cylinder (1) is connected to the sample source in the form of an automatic sampling system or sample changer (62), or a pipe (60) through which the stream of the tested (monitored) sample flows, or an airlock (50) on the pipe (60), while through the hole (16) and the hose (44) it is connected to the waste channel (61). The cylinder (1) also has the gas hose (47) embedded in the hole (14) to equalise the pressure in the hydraulic system. Preferably, the hose (47) is open to the environment and provides a source of atmospheric air (63), but it can also provide access to a specific gas by connecting it to a gas source (63), for example in the form of a high-pressure cylinder equipped with a pressure gauge working in the range of pressures close to atmospheric pressure, preferably operating in the range of slight overpressure. The hoses (99,41,44,47) are made of a flexible but rigid chemically inert material, for example perfluorinated polymers, preferably of poly(tetrafluoroethylene) (PTFE), fluorinated ethylene-propylene (FEP) or NAFION (copolymer of tetrafluoroethylene and perfluorinated oligovinyl ether terminated with a sulfone group). The hoses (99,41,47,44) have an outlet directly to the cylinder (1) through the holes (10,12,14,16) in its wall respectively, where the hoses are sealed. The diameter of the holes (10,12,14,16) is equal to the outer diameter of the hoses (99,41,47,44), which is 0.4-2.0 mm, preferably 0.8 mm. The hoses (99,41,47,44) are seated in the holes (10,12,14,16) tightly detachably, preferably sealing the connection with a flexible waterproof tape, for example with a waterproof flexible double-sided foam tape with an adhesive mass, 1-3 mm thick, preferably 1.110 mm. What is extremely important, the holes (10,12,14,16) are located in different parts of the cylinder (1) to ensure that the fluid or gas can be transferred to each of these hoses separately. The distance between the projections of the positions of the holes (10,12,14,16) on the axis of the cylinder (1) in variant I is 2-10 mm, preferably 5 mm. The spacing of the holes (10,12,14,16) by 5 mm is beneficial because it allows for minimisation of the dimensions of the cylinder (1) while ensuring a sufficiently large working volume for the determinations. Using a 5 mm spacing and hoses with an internal diameter of 0.8 mm, the maximum volume of the working space between the pistons (2), ensuring contact with a single hole (10,12,14,16) in the cylinder (1) with a diameter of 12 mm, equals 1040 μl, which corresponds to the opening of the pistons (2) to 9.2 mm, and the maximum working volume for moving the pistons in hydraulically and pneumatically neutral conditions, it is 470 μl, which corresponds to the opening of the pistons (2) at 4.2 mm, i.e. the opening ensuring exposure to a single hole (10, 12, 14, 16) in each position of the pistons (2) in the system.

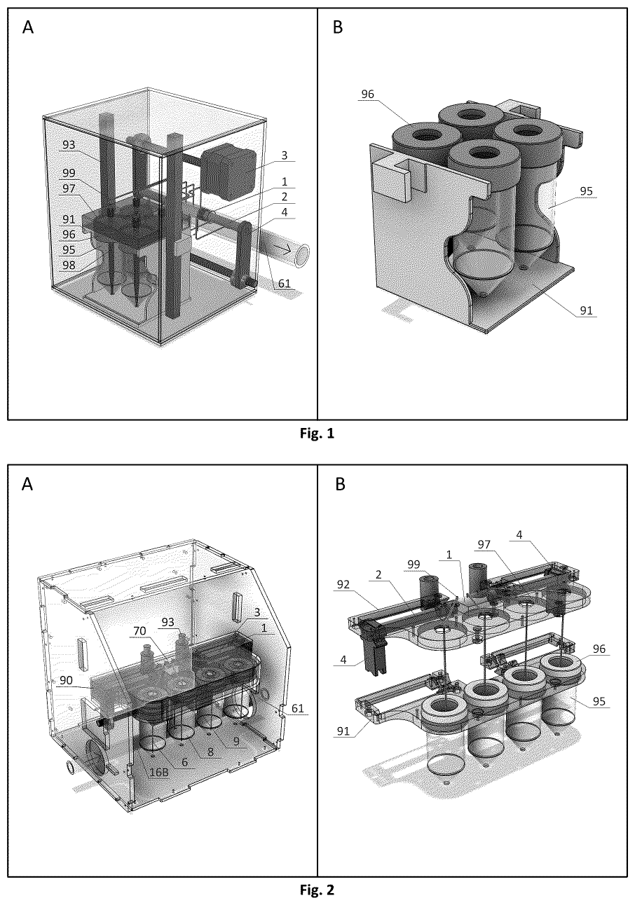

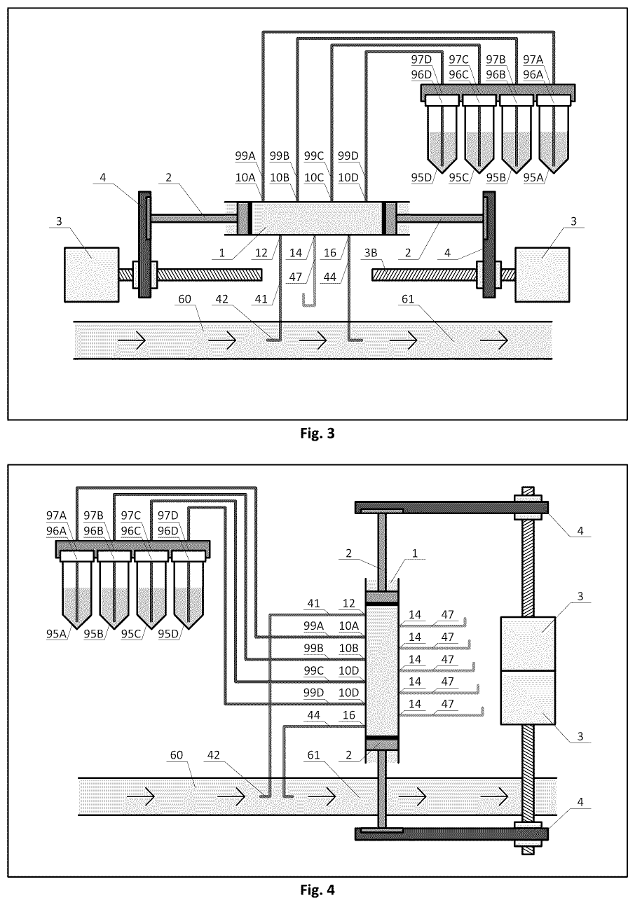

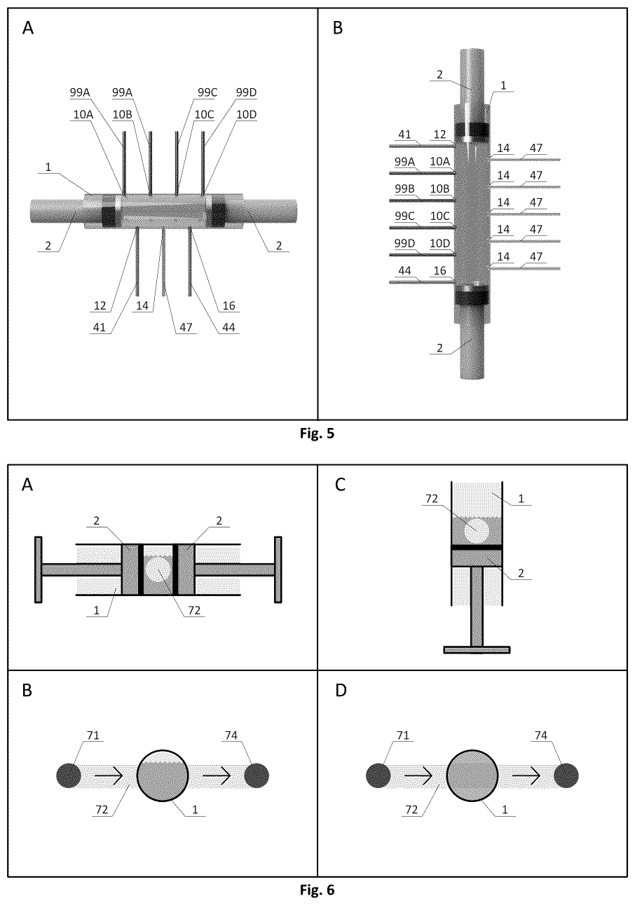

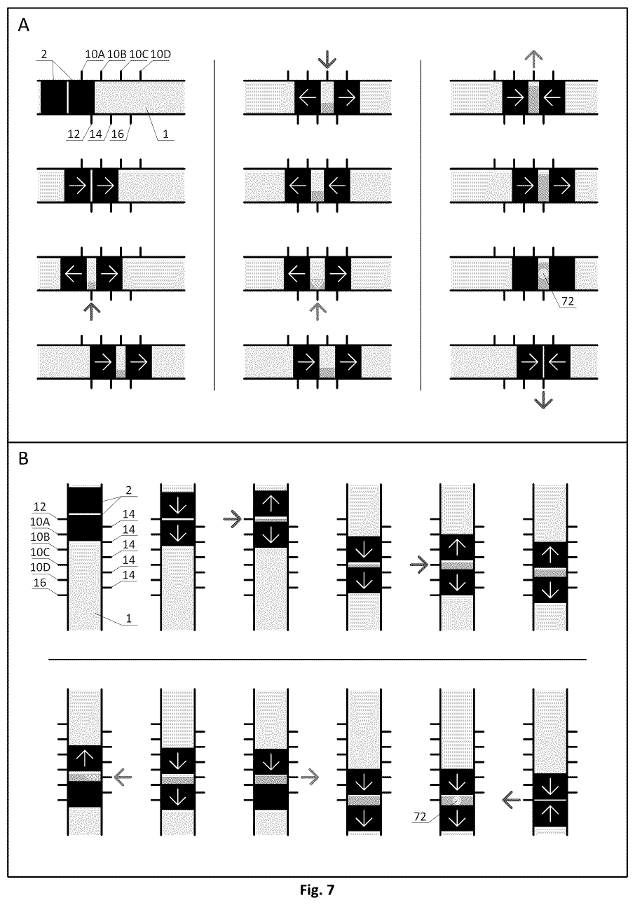

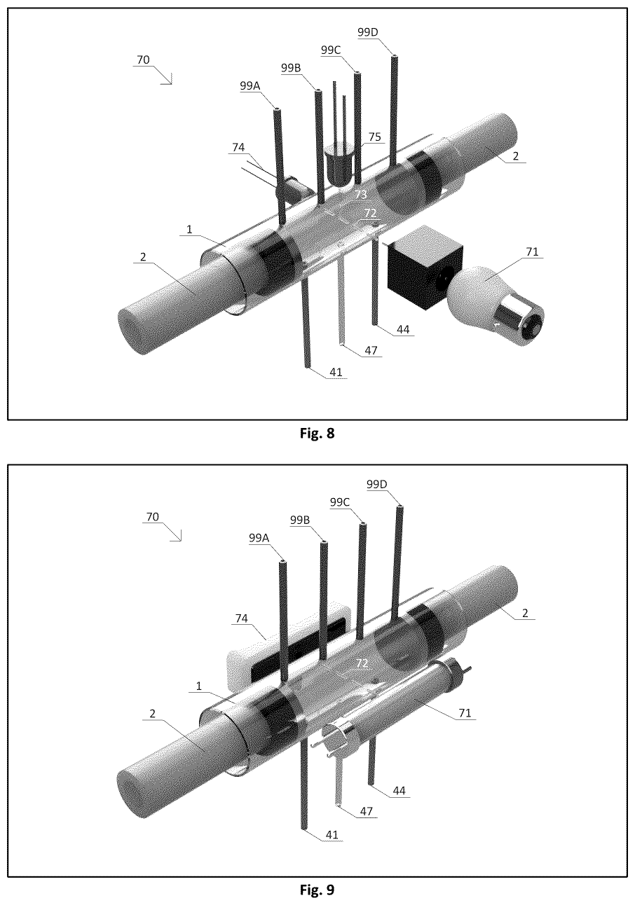

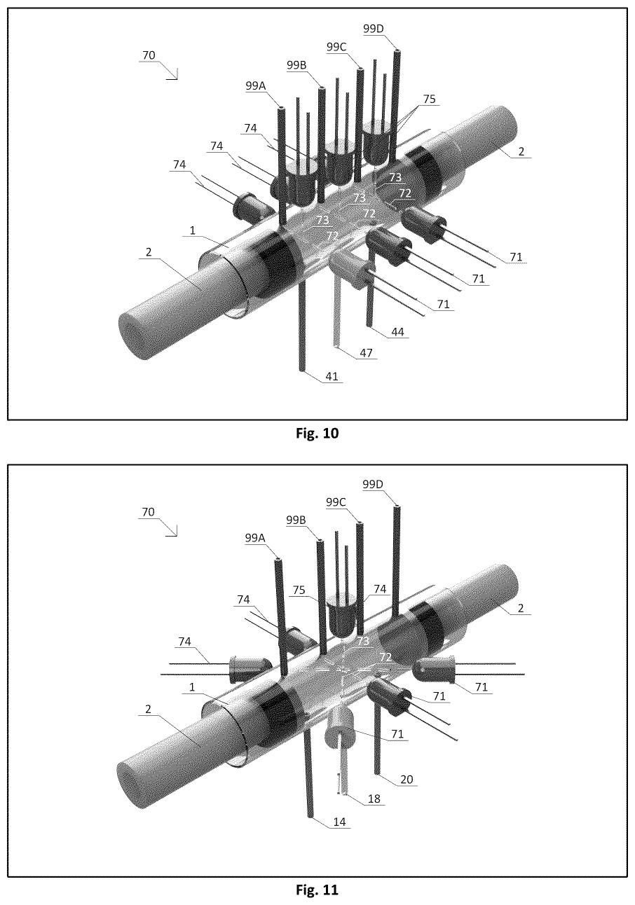

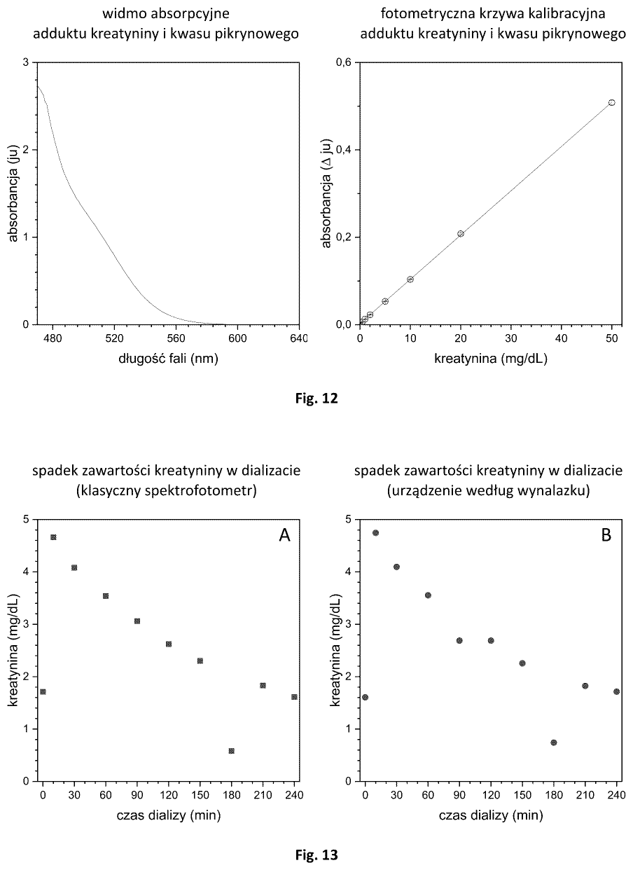

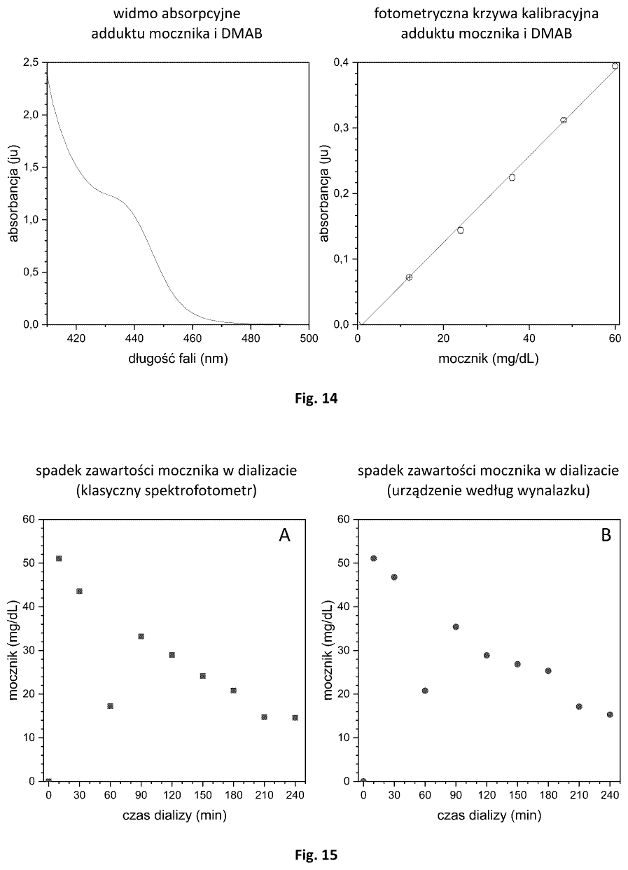

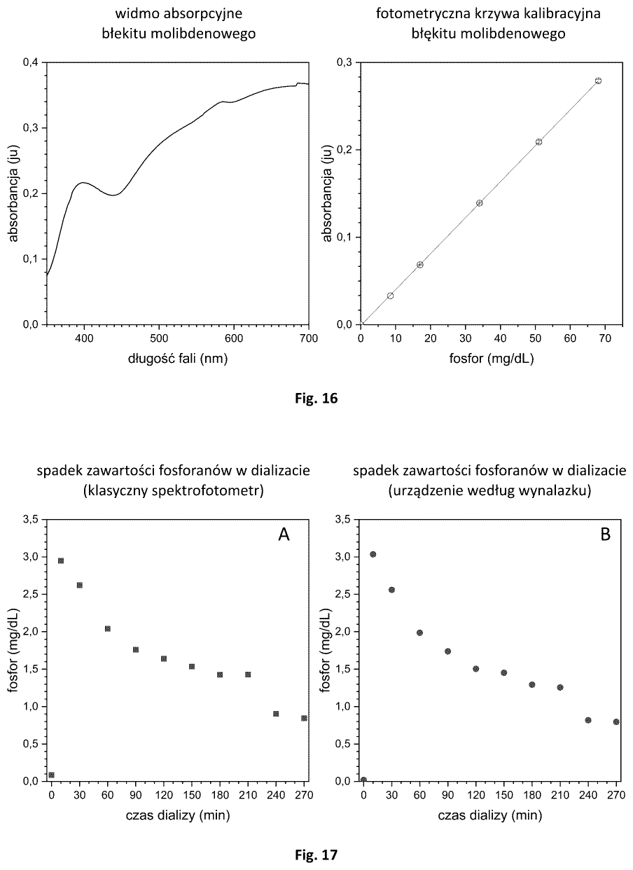

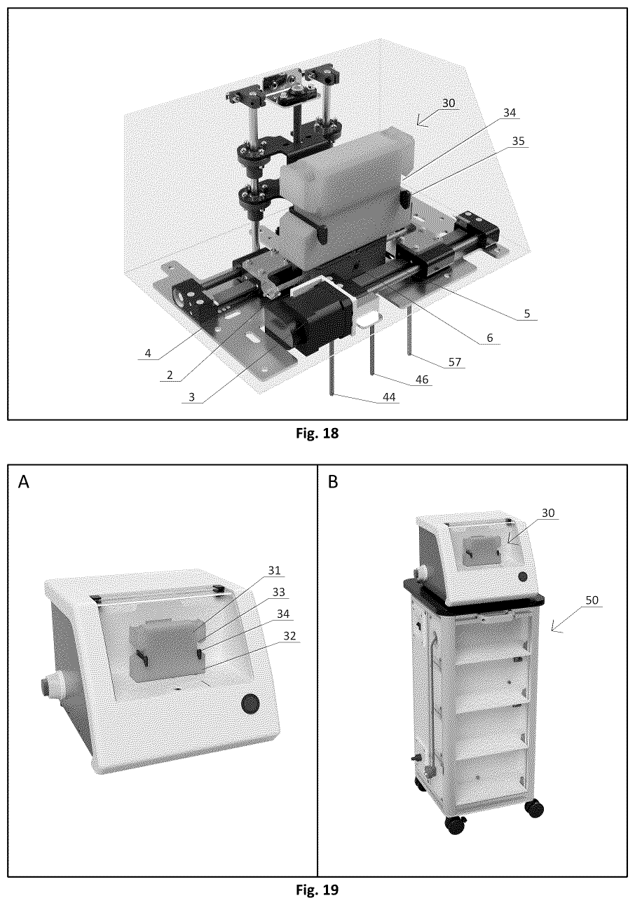

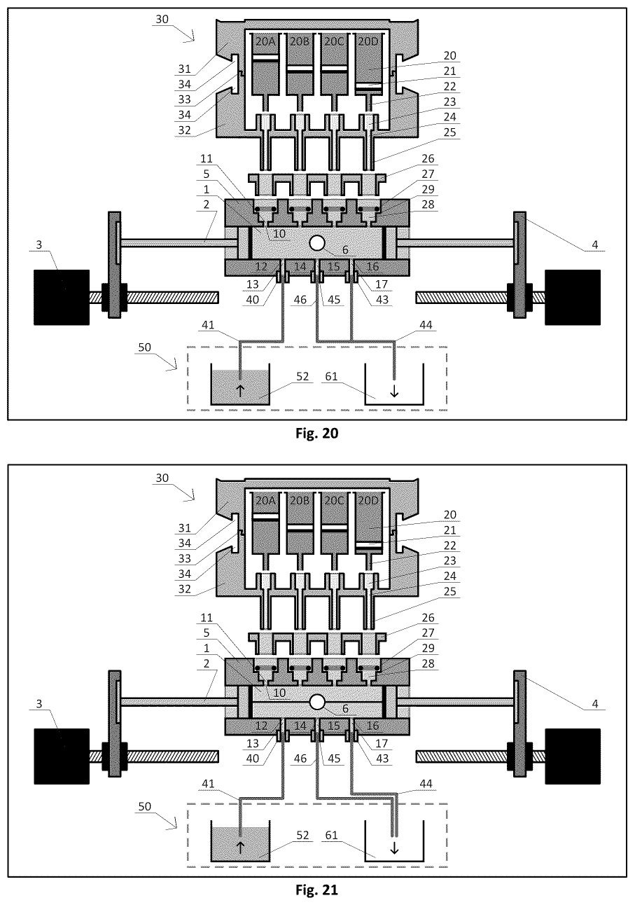

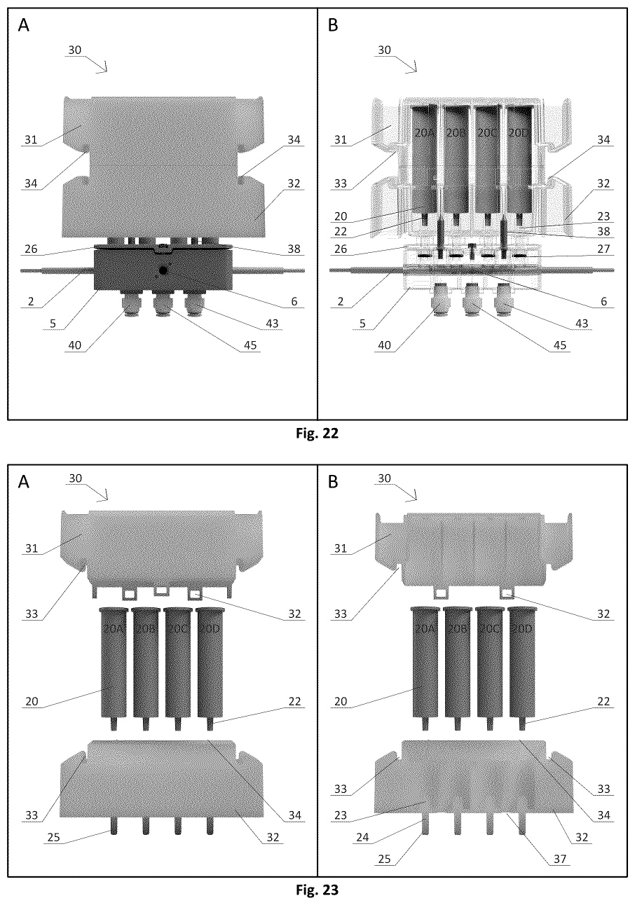

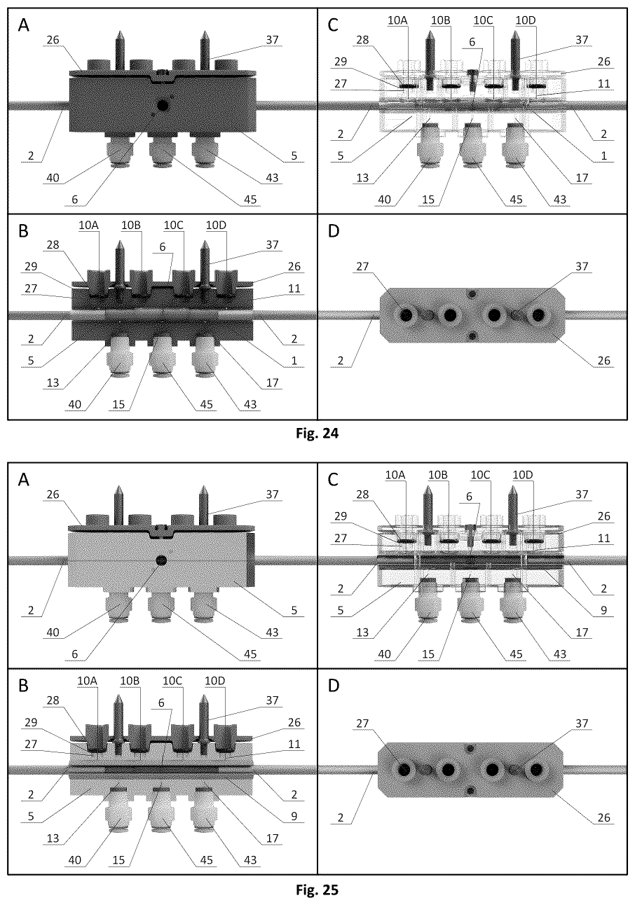

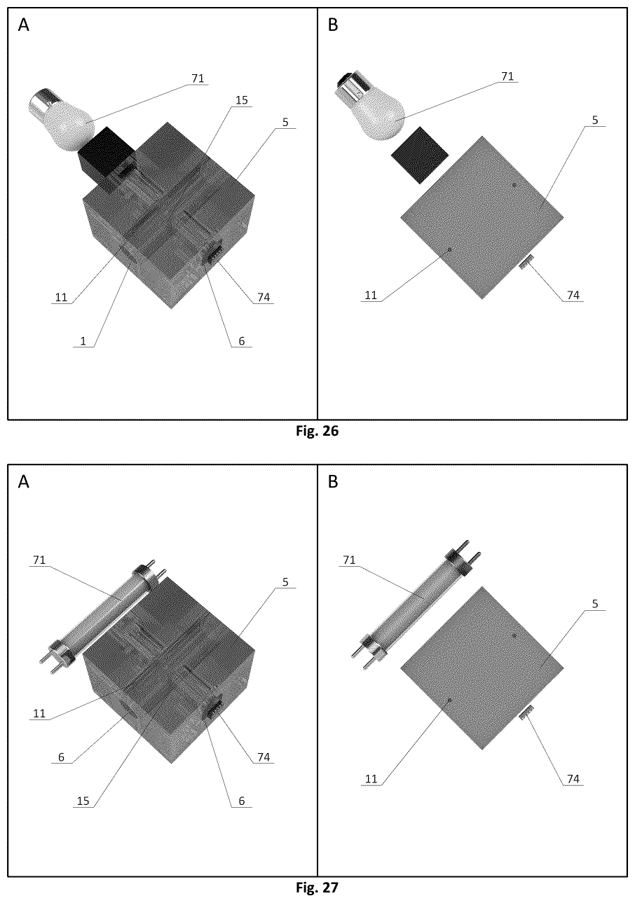

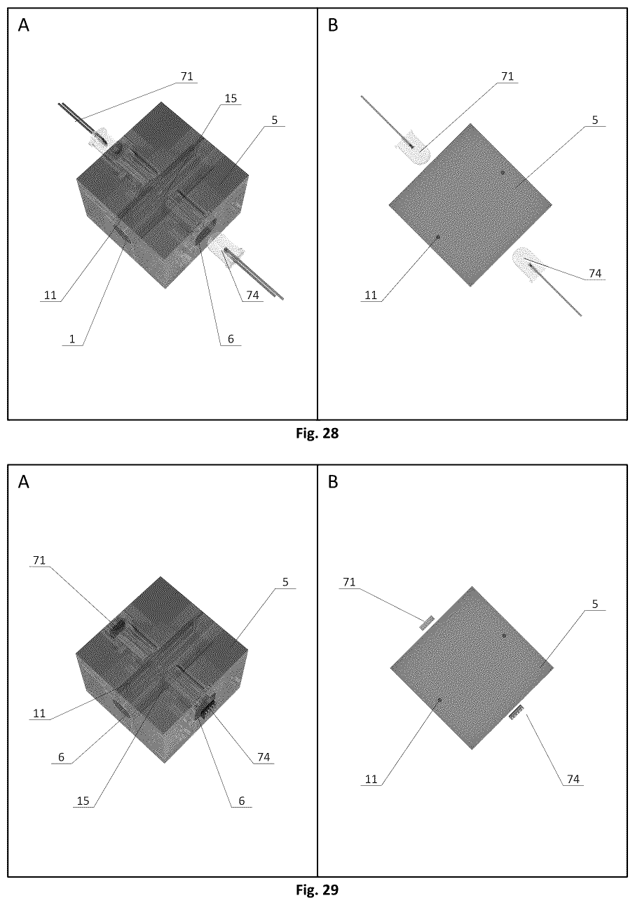

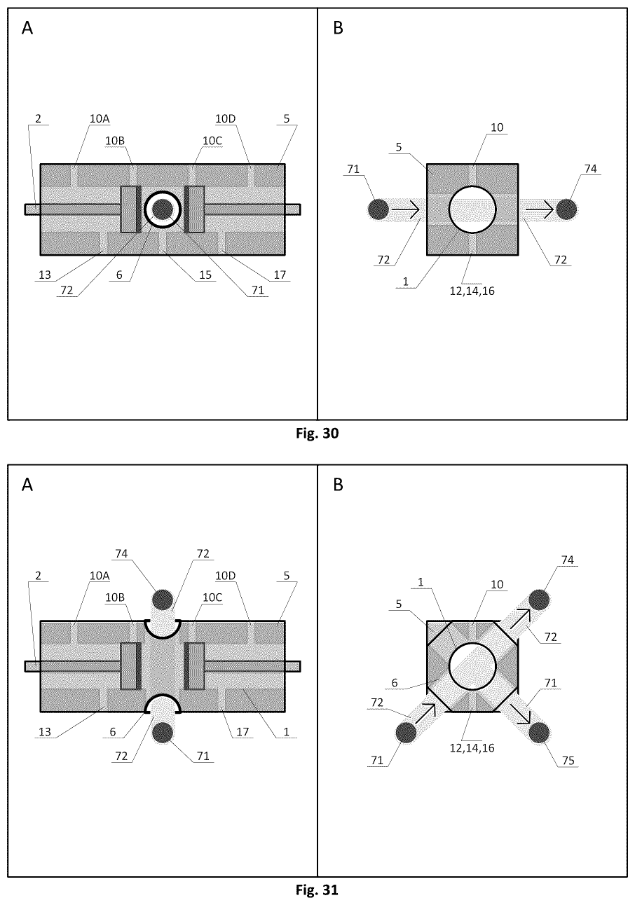

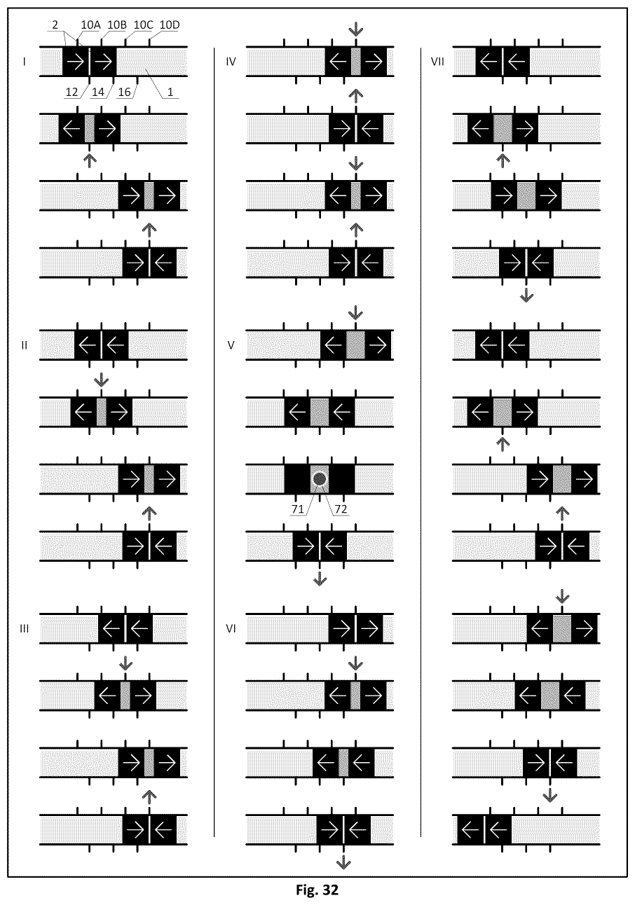

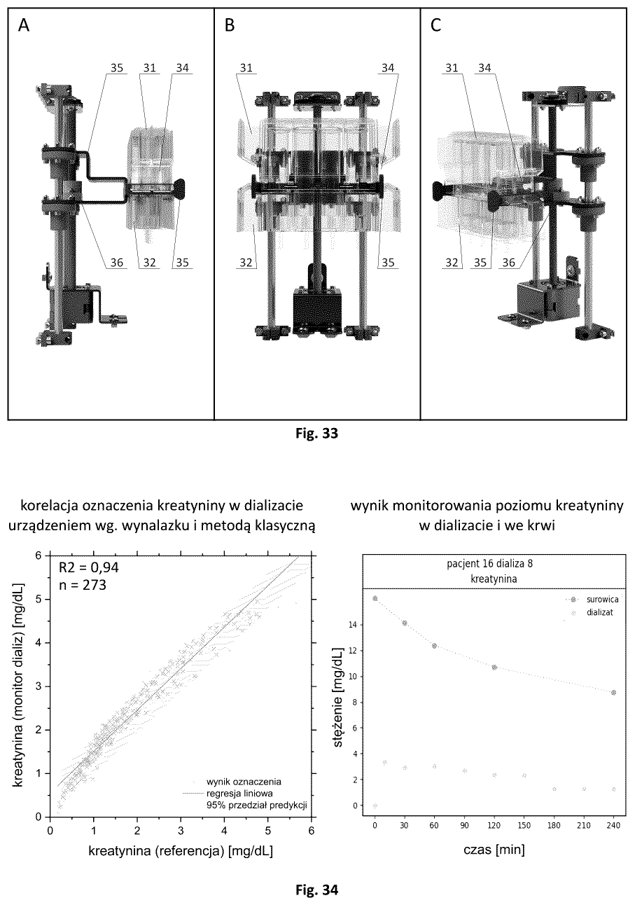

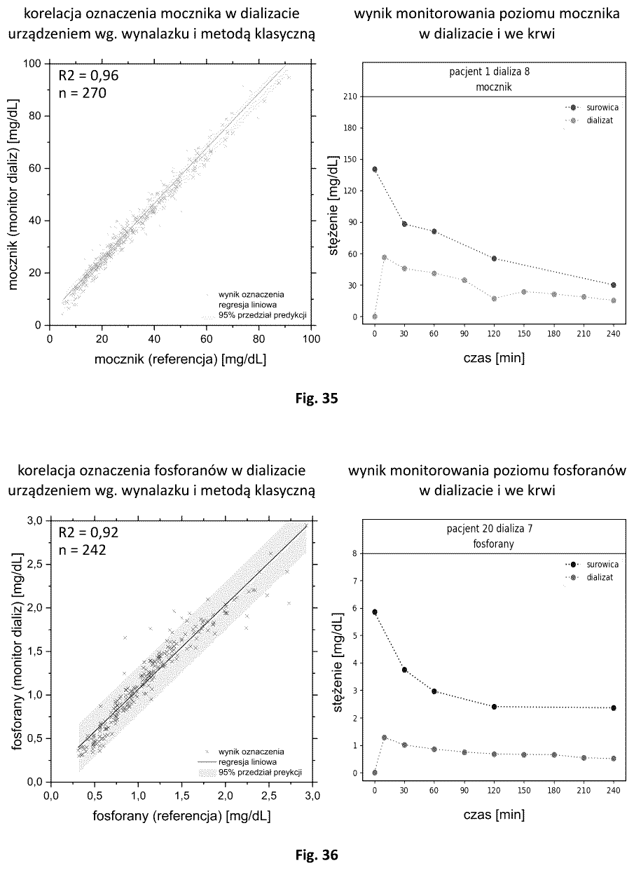

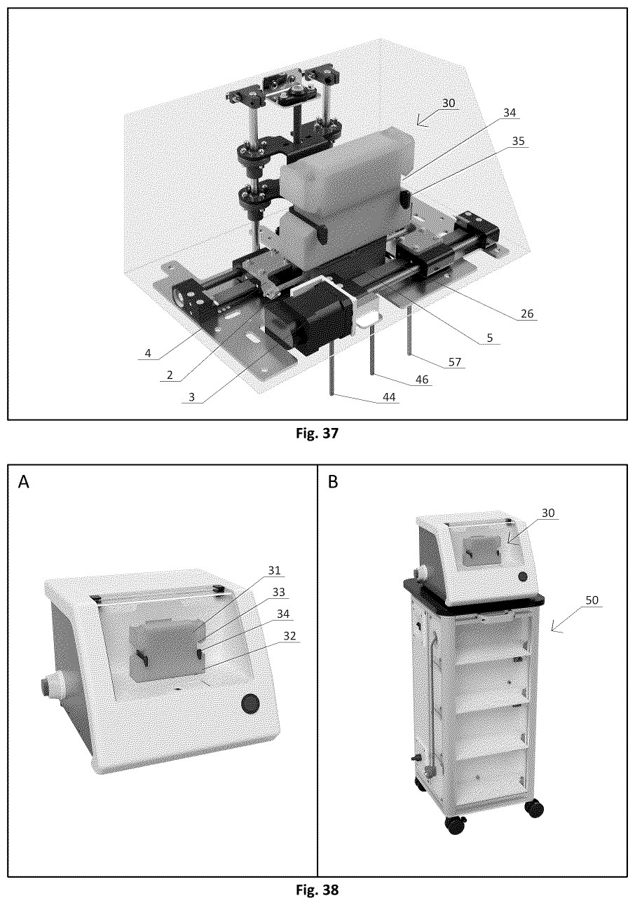

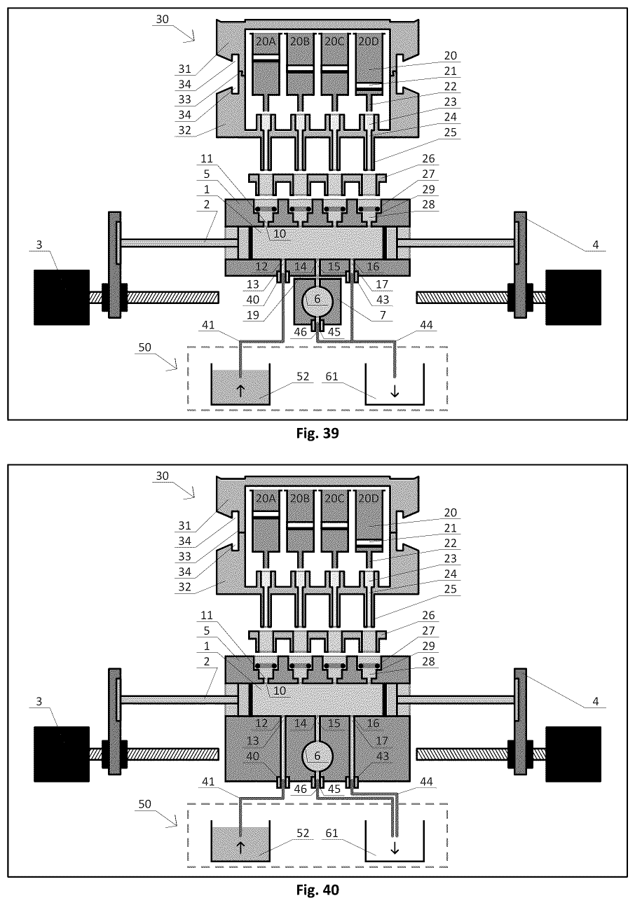

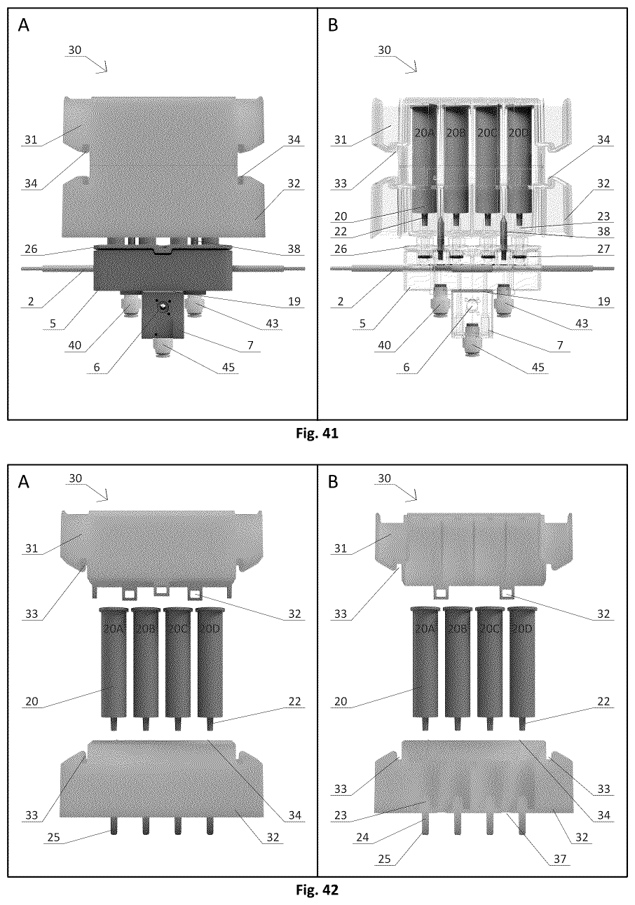

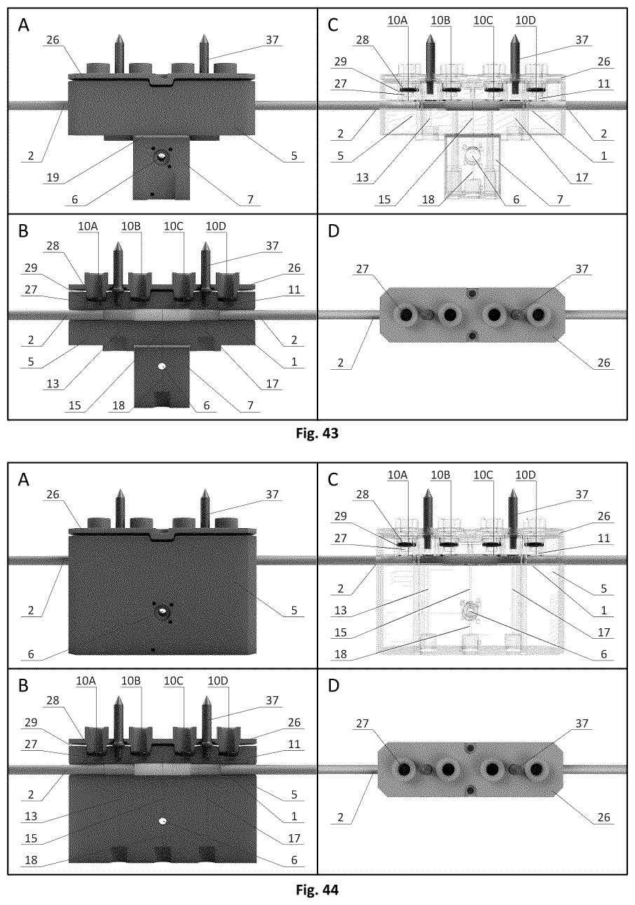

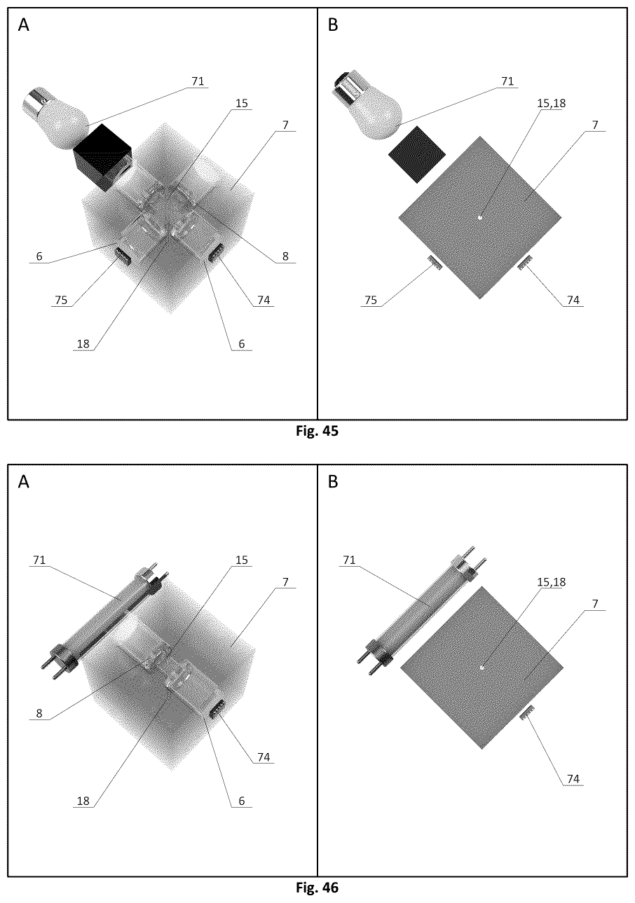

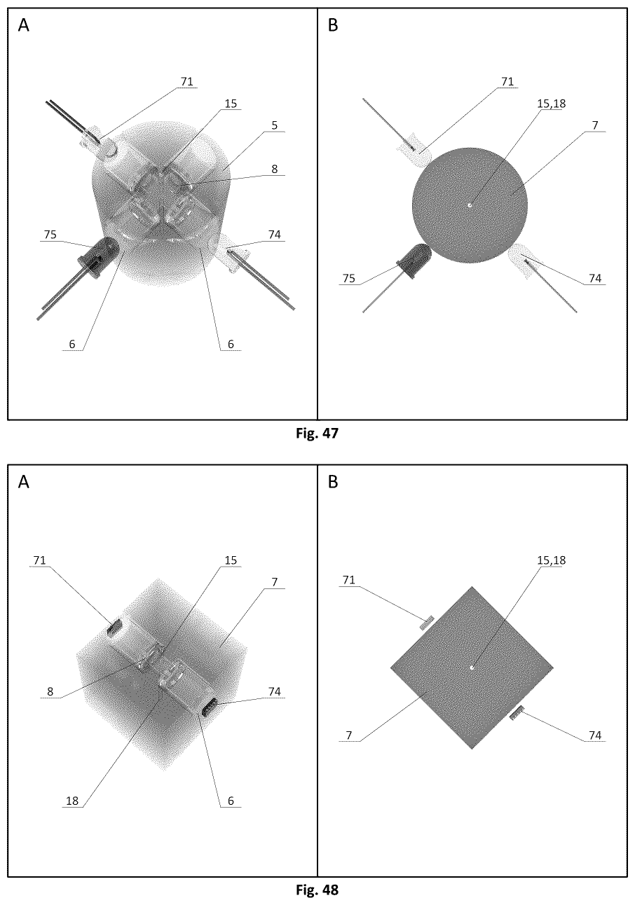

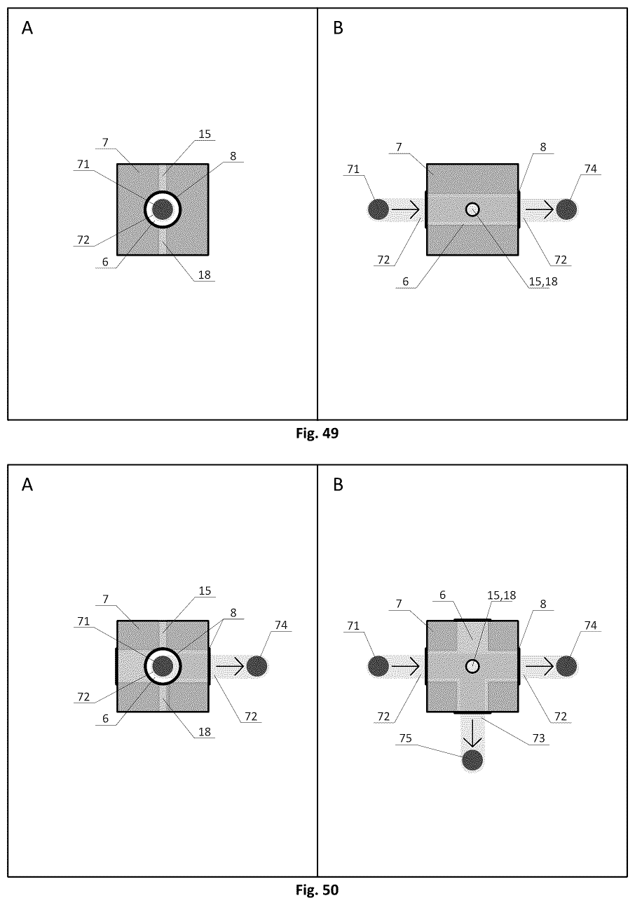

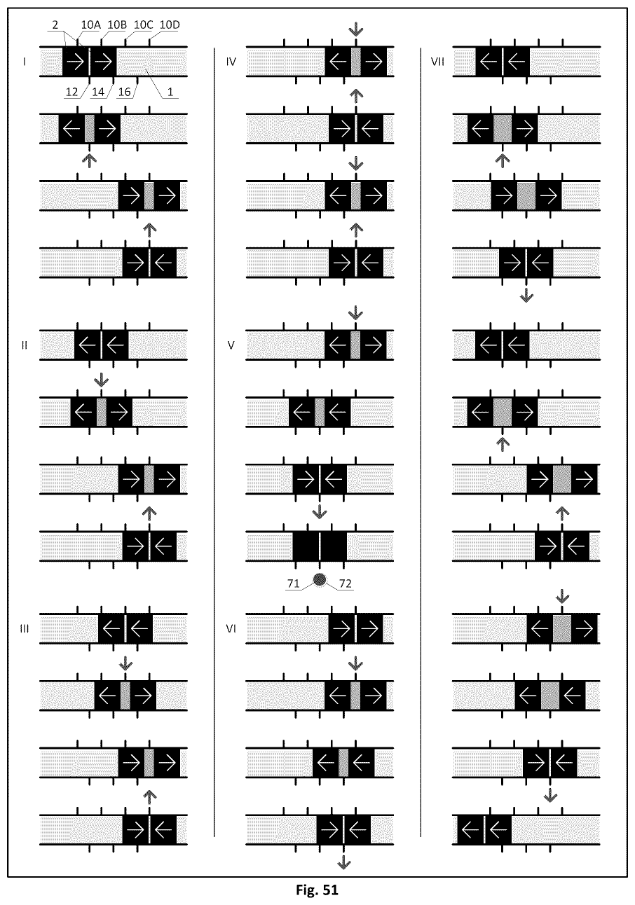

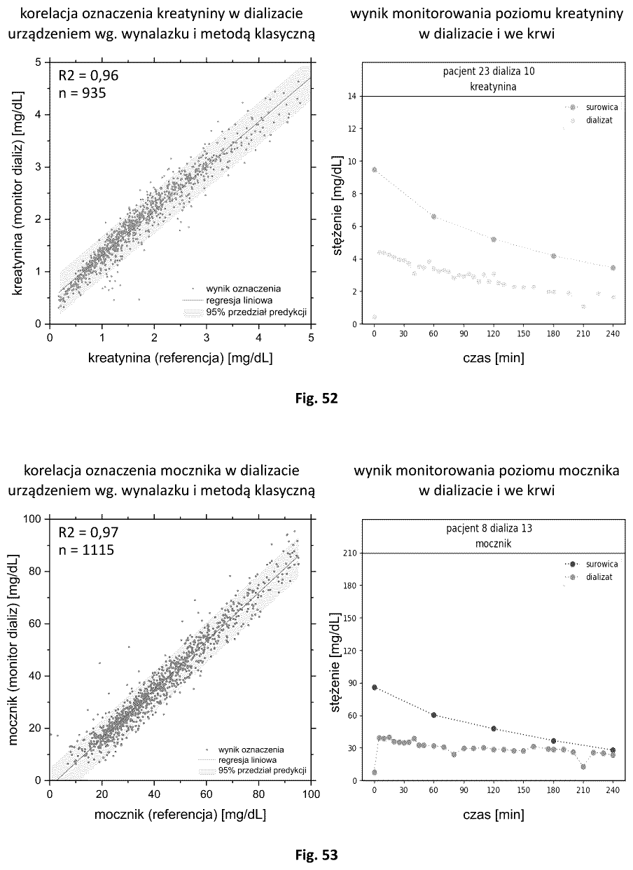

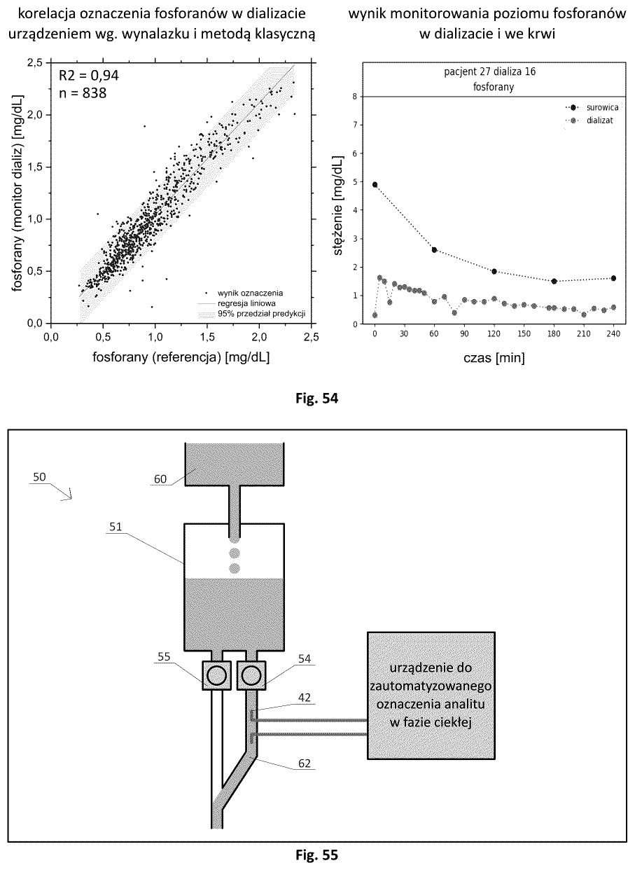

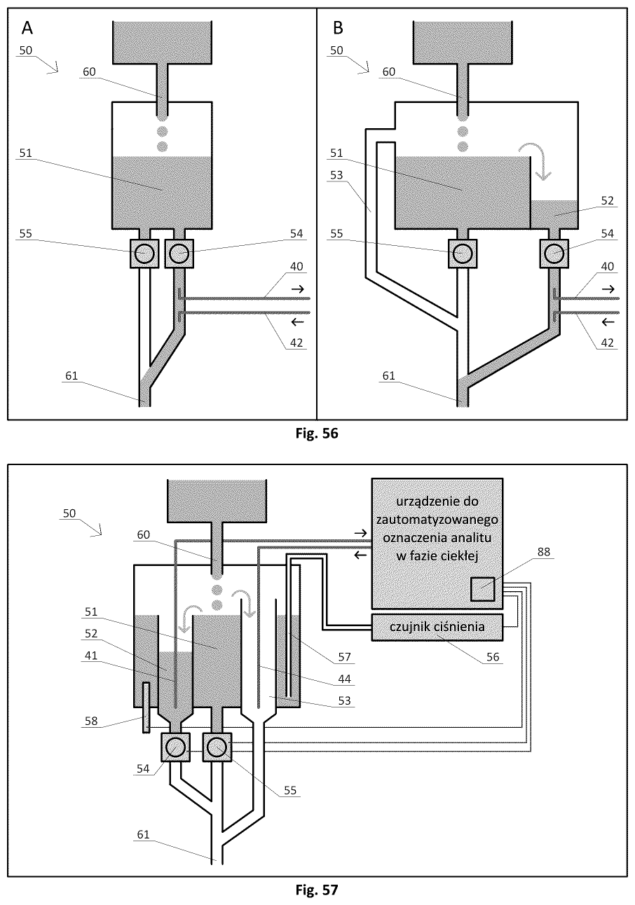

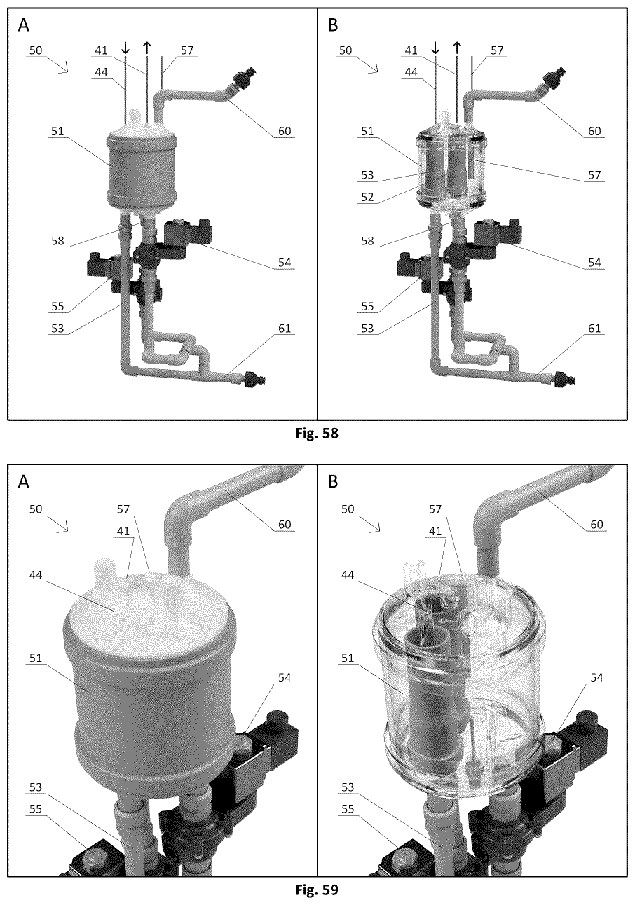

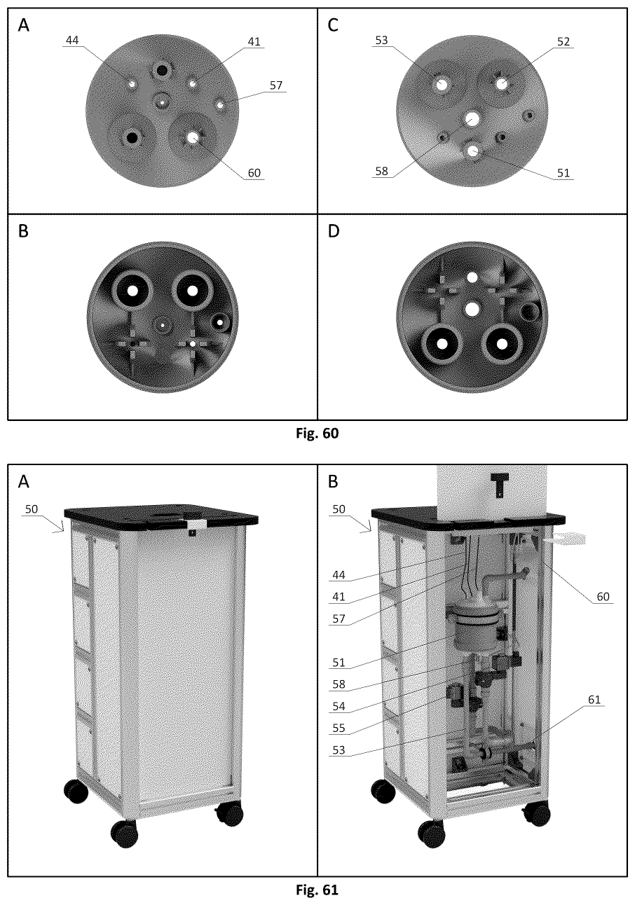

In variant I, the cylinder (1) can be placed in the device at any angle to the surface, but the horizontal orientation (FIG. 3, FIG. 5A, FIG. 6A-6B, FIG. 7A) and vertical orientation (FIG. 4, FIG. 5B, FIG. 6C-6D, FIG. 7B) are considered particularly preferred.

The cylinder (1) in a horizontal orientation, according to the invention, provides the possibility of minimising the risk of contamination of the reagents stored in the reservoirs (95) by placing the outlets of the hoses (99) connecting the reservoirs (95) with the cylinder (1) in the holes (10) located on the top of the cylinder (1), i.e. preferably in a way that the diameters of the holes (10) lie in a vertical plane passing through the axis of the cylinder (1) at its intersection with the side surface of the cylinder (1) located above its axis. Thanks to that, the risk of accidental pumping the reaction solution into the reservoirs (95) is practically impossible. In turn, the holes (12,14,16) connecting the cylinder (1) with the sample source (50,60,62), the gas source (63) and the waste channel (61) are located at the bottom of the cylinder (1), i.e. preferably in a way that the diameters of the holes (12,14,16) lie at the intersection of the vertical plane passing through the axis of the cylinder (1) with the side surface of the cylinder (1) below its axis. Such an orientation of the hole (12) allows the tested solution to be pumped into the cylinder (1) from the bottom, i.e. from a different direction than the flow of reagents coming from the reservoirs (95), which additionally reduces the risk of their contamination. What's more, it allows for efficient rinsing the cylinder (1) with larger portions of the tested solution. Placing the hole (14) at the bottom of the cylinder (1) enables efficient mixing of the reaction solution with a portion of gas drawn from the bottom. In turn, placing the hole (16) at the bottom of the cylinder (1) allows for efficient removal of all post-reaction solution from the cylinder (1) to the waste channel (61) (FIG. 7A). According to the invention, the holes (10) in the upper part of the cylinder (1) alternate with the holes (12,14,16) in the lower part of the cylinder (1). The preferred sequence of holes in any direction in the cylinder wall (1), using four reservoirs (95), is as follows: 10A, 12, 10B, 14, 10C, 16, 10D.

The cylinder (1) in an alternative vertical orientation, according to the invention, provides the possibility of accelerating the process of analyte determination in the tested solution thanks to the use of an appropriate sequence of holes (10,12,14,16) allowing for the subsequent stages of the determination with unidirectional progressive movement of the pistons (2) in the cylinder (1). The cylinder (1) has more than one hole (14) with a hose (47) for equalising the gas pressure, preferably the holes (14) are located between the individual holes (12,14,16) to facilitate the process of refilling or reducing the amount of gas in the cylinder (1). The outlets of the hoses (99,41,47,44) may exit on either side of the cylinder (1), but it is preferable to arrange the holes (10,12,16) on one side and the holes (14) on the other side. The risk of contamination of the reagents in the reservoirs (95) is eliminated thanks to the method of pumping gas into the hoses (99) after the uptake of the reagents. Preferable sequence of the holes in the wall of cylinder (1) from top to bottom, using four reservoirs (95), is as follows: 12, 14, 10A, 14, 10B, 14, 10C, 14, 10D, 14, 16. Such a sequence of holes (10,12,14,16) allows for easy injection of the tested solution into the cylinder (1), and every rinsing of the cylinder (1) with the tested solution before the measurements. Then, the pistons (2) moving in one direction downwards (FIG. 7B) are able to uptake the appropriate reagents from the reservoirs (95) each time refilling or reducing the amount of gas between the pistons (2). It is also possible to mix the reaction solution with the gas from the holes (14) in multiple regions of the cylinder (1). Placing the hole (16) at the end of the sequence, at the bottom of the cylinder (1), allows for efficient removal of the entire post-reaction solution from the cylinder (1) to the waste channel (61), and removal of the solution after washing the cylinder (1) between the determinations.

Variant I with a horizontal cylinder (1) is designed mainly for flow testing of changes of the analyte concentration in the stream of the tested solution, where successive portions of the solution are sampled at given intervals directly into the cylinder (1), and the sampling is preceded by washing the cylinder (1) with a larger portion of the tested solution, which is available in large quantities without restrictions, for example, to determine the level of toxins in the dialysate stream. In such arrangement, the sampling system is then the tip (42) or extension of the hose (41) connecting the cylinder (1) with the sample source (50,60,62), which in this case is a pipe (60) through which the dialysate stream is pumped, curved in the opposite direction to the stream of the tested solution. It is also possible to sample with a hose (41) from the accumulation reservoir (52) of the airlock (50) located on the channel (60) with sample stream. On the other hand, variant I with a vertical cylinder (1) can be used to determine the samples using any sampling system, preferably as described above or a classic sample changer (62) for stationary measurements.

According to the invention, the device comprises at least four reservoirs (95A,95B,95C,95D) for the reagents, oriented in a normal manner, i.e. with the outlet pointing upwards. The first reservoirs (95A) is filled with a portion of the analyte standard solution, which is used to carry out calibration measurements (ST). Two subsequent reservoirs (95B,95C) are filled with chemical reagents necessary to carry out the specific reaction, which often cannot be stored in one vessel due to their mutual chemical instability (R1,R2). It is also possible to use these reagent reservoirs for various specific reactions, if there is such an analytical possibility. The last reservoir (95D) can act as a backup vessel, can store another reagent, remain empty or be filled with a portion of a matrix solution free of analyte, which can be used for washing the cylinder (1) and performing calibration measurements (MATRIX). According to the invention, it is possible to use more reservoirs in the normal cartridge (90) (e.g. 95E,95F,95G, etc.) for further chemical reagents, if necessary, but four reservoirs, for current applications, is the optimal number to minimise the dimensions of the cartridge. Each of the reservoirs (95A,95B,95C,95D) is connected to the cylinder (1) using its own dedicated hose (99A,99B,99C,99D) which is embedded in its own dedicated hole (10A,10B,10C,10D) in the wall of the cylinder (1).

In variant I, the reservoirs (95) are preferably shaped like vials with conical bottoms, thanks to which it is possible to uptake the liquids efficiently even with their small amount in the reservoirs (95). Preferably, commercially available eppendorf vials for centrifuges are used as the reservoirs (95). The volume of the reservoirs (95) can vary, but must be large enough to provide enough portion of the reagents for the planned series of determinations (e.g. 20-50 measurements), while being relatively small to ensure compactness of the device. According to the invention, the reservoirs (95) have a volume of 10-50 ml, preferably 25 ml, with a height in the range of 50-100 mm, preferably 78 mm, which is the size of a 25 mm eppendorf vial including the cap. The reservoirs (95) has a closure (96) at the top, preferably in the form of a cap or plug, with an opening of a diameter of 4-20 mm, preferably 6-14 mm, enabling the insertion of a hose (99) for drawing the reagents. Optionally, the opening in the closures (96) is closed with a septum membrane, and the closures (96) themselves have an additional opening with a diameter of less than 1 mm to equalise the pressure in the reservoirs (95) when drawing the solutions.

The reservoirs (95) with closures (96) are rigidly seated in round sockets (97) that receive the closure (96) from the bottom. The inner diameter of the sockets (97) corresponds to the outer diameter of the closures (96).

According to the invention, the solutions are taken from the reservoirs (95A,95B,95C,95D) by means of appropriate hoses (99A,99B,99C,99D). Plastic two-piece through-nuts are used over the closure openings (96) to ensure hose rigidity and their immobility. The length of the hoses (99) is larger than the depth of the reservoirs (95) to ensure that the ends of the hoses are self-laid on the bottom of the reservoirs (95). In a preferred embodiment, the cups (96), as well as the nuts and hoses (99), are permanently seated in the sockets (97). Then, the reservoirs (95) are mounted by screwing them from the bottom into the cups (96) placed in the sockets (97).

Optionally, the ends of the hoses (99A,99B,99C,99D) are embedded in dedicated tips (98) enabling puncturing the septum in the closures (96), which are inserted coaxially into the reservoirs (95). The tips (98) are cylindrical in shape with a diameter of 4-8 mm, preferably 6 mm. To ensure the ease of puncturing the septum, the diameter of the tips (98) decreases towards the bottom. The outlets of the hoses (95) are slightly above the lower edge of the fittings (98), preferably 0-2 mm above this edge, and the inner diameter of the lower edge of the tips (98) is equal to the outer diameter of the hoses (99). Each of the tips (98) is made of a conductive material (e.g., carbon doped PP) and is connected to the main controller (88) which allows the impedance of the tips (98) to be measured and the fluid level in each reservoir (95) to be determined while the device under operation. Knowing the level of the reagents is needed for precise uptake when conducting the reactions. The tips (98) are rigidly seated from below in the centre of the circular sockets (97) receiving the closures (96) of the reservoirs (95) inserted into these sockets from below. The length of the tips (98) is matching the size of the reservoirs (95) in such a way that the distance between the lower edge of the hoses (99) and the bottom of the reservoirs (95), after they are properly seated in the socket (97), equals 0.5-2.0 mm, preferably 1.0 mm. Thanks to this, the outlets of the hoses (99) are reproducibly positioned inside the reservoirs (95), and it is also possible to uptake the reagent even at a very low level in the reservoir (95). In the preferred variant of using commercially available eppendorf vials (95) with a volume of 25 ml, height of 78 mm and a conical bottom, the length of the tips (98) is 74-76 mm, taking into account the thickness of the bottom of the vial and 0.5 mm spacing between the surface of the socket (97) and the surface of the caps (96).

The device, according to the invention, in variant I, is equipped with a replaceable normal cartridge (90) containing elements requiring replenishment between the determinations, and in a preferred embodiment, consumables requiring frequent replacement. The cartridge (90) is in the form of a container having a frame (91) and positioning means for the reservoirs (95). The cartridge (90) is inserted into the device bed provided with guides (93) for receiving the frame (91) of the cartridge (90). The device bed also has means (94) for locking the cartridge (90) in the correct in position, preferably in the form of a lift, an automatic lift, a lock with a release mechanism, a magnet assembly, an electromagnet assembly, or a closable door. Particularly advantageous is the variant with an automatic lift (94) driven by a dedicated stepper motor controlled by the main controller (88), because it allows to automate and standardise the process of placing the cartridge (90) into the device, and to exclude the possible human error. In addition, the cartridge (90) has reservoirs (95) for chemical reagents. In a preferred variant, the cartridge (90) has four reagent reservoirs (95A,95B,95C,95D) containing sequentially: an analyte standard, a first chemical, a second chemical, and an analyte-free reference matrix solution. It is possible to use a cartridge (90) with more reservoirs (95) if there is an analytical need, which requires appropriate adjustment of the cartridge structure (90), the device bed and the location of the sockets (97), as well as the hydraulic system and the number of holes (10,12,14,16) and their arrangement of the cylinder (1).

In various embodiments of the device, in variant I, the cartridge (90) and the bed in the device may take different forms depending on the number of components placed in the cartridge and the method of drawing solutions (a variant with the tips (98) housing the outlets of the hoses (99) introducing them to the reservoirs (95), or a variant only with hoses (99) introduced to the reservoirs (95)).

The normal cartridge (90), in a simplified version (FIG. 1), acts only as a carrier for the reservoirs (95), preferably four reservoirs (95A,95B,95C,95D), facilitating only the replenishment/replacement of the reagents for carrying out determinations and calibration. In this variant, according to the invention, the bed of the device in its upper plane has a set of four sockets (97), optionally with tips (98) for the hoses (99), coaxial with the reservoirs (95A,95B,95C,95D) in the cartridge (90). The tips (98) puncture the septum membranes in the closures (96) of the reservoirs (95) when inserting the cartridge (90) into the device. Proper, parallel placement of the cartridge (90) in the device is ensured by the guides (93) receiving the

The normal cartridge (90), in the preferred version (FIG. 2), contains also the cylinder (1) and pistons (2), as well as other necessary components for the efficient operation of the device, i.e. connectors (4), hoses (99), tips (98) in the sockets (97), hose/hoses (47) and fragments of the hoses (41,44). The advantage of this solution is the easier access to the cylinder (1) and pistons (2), thanks to which it is possible to treat it as a disposable reactor and exchange it between the successive measurement series. Both horizontal and vertical orientation of the cylinder (1) is possible. The pistons (2) are embedded in the connectors (4) detachably connecting to the positioning nuts on the lead screws of the stepper motors (3). When inserting the cartridge (90) into the device, the connectors (4) located on the fully extended pistons (2) overlap the fully extended positioning nuts. In turn, as the cartridge (90) is ejected, the connectors (4) slide off the positioning nuts. Optionally, the cartridge (90) consists of two interconnecting structural elements, the first (91) comprising a frame, a space for housing the cylinder (1) with pistons (2) and connectors (4), hoses (99), hose/hoses (47), sockets (97), and guides with a stabilising element receiving a second structural element (92) containing the positioning elements for the reservoirs (95). The elements (91,92) are detachably connected to each other in a unique way, ensuring the coaxiality of the reservoirs (95) and the sockets (97). Alternatively, the cartridge (90) is one piece and includes a frame (91), a space to accommodate the cylinder (1) with pistons (2) and connectors (4), hoses (99), hose/hosed (47) and sockets (97). In this variant, preferably the reservoirs (95) are permanently placed in the sockets (97), and the cartridge (90) is a disposable element that can be regenerated. This simplifies the operation of the device and facilitates the work of the operator. Alternatively, the reservoirs (95) are placed in the sockets (97) manually prior to measurement.

Regardless of the construction of the cartridge (90), the remaining hoses (41,44) used to draw the tested solution from the sample source (50,60,62) and to remove the post-reaction solution to the waste channel (61), i.e. connecting the cylinder (1) in the cartridge (90) to them, must be divided into at least two sections joined together when placing the cartridge (90) in the device using standard connectors. Any type of connectors can be used, preferably two-element connectors having compatible elements: male and female. Thus, the hose (41) is divided into two sections, one of which (41A), sealed in the hole (12) of the cylinder (1), is located in the cartridge (90) and ends with a connector (41B) (male or female), and the other (41D) starts with a connector (41C) (compatible with the connector (41B)) embedded in the bed receiving the cartridge (90), and continues in the body of the device connecting to the sample source (50,60,62). Similarly, the hose (44) is divided into two sections, one (44A) terminating in a male or female connector (44B) located in the cartridge (90) and the other (44D) starting with a connector (44C) (compatible with the connector (44B)) in the bed receiving the cartridge (90), and continues to the waste channel (61).

The flow of liquid in the hydraulic system is forced by the mutual movement of the pistons (2) inside the cylinder (1), generating overpressure or underpressure of gas in a specific part of the hydraulic system, which forces the movement of the liquid to balance the pressure in the system. It should be noted that the reservoirs (95), the sample source (50,60,62) and the waste channel (61), connected to the cylinder (1), are open to the outside environment or are open to a protective atmosphere with a gas supply, i.e. are capable of equalising the gas pressure. The area of influence of the generated gas pressure changes in the hydraulic system, understood as the whole system connected to the cylinder (1), is limited by the position of the pistons (2) which can move freely inside the cylinder (1). The fluid flow in the system is thus controlled pneumatically, not hydraulically as in the prior art. A change in the relative position of the pistons (2) in the cylinder (1) generates the mentioned changes in the gas pressure in the hydraulic system. Refill or reduction of the amount of gas is possible only when the hole (16), supplying gas directly to the interior of the cylinder (1) through the hose (47), is located in the working space between the pistons (2), and then the gas is sucked in or pushed out of the cylinder (1) by the appropriate mutual movement of the pistons (2), i.e. when the pistons (2) move apart, the gas is sucked into the cylinder (1), and when the pistons (2) are pushed together, the gas is pushed out of the cylinder (1). On the other hand, if in the working space between the pistons (2) there is an outlet of one of the hoses (10,12,14,16), the movement of the liquid is generated in the direction that balances the pressure changes, i.e. when the pistons (2) are moved apart, the liquid is sucked into the cylinder (1), and when the pistons (2) are pushed back, the liquid is pushed out of the cylinder (1). It should be emphasised that in the variant with the horizontal cylinder (1), the gas cannot be pushed out through the hole (14) because the solution would be pushed out first. In this case, the removal of an excess gas is managed by any of the holes (10), then the gas leaves the system through a reservoir (95). It is worth noting that when the pistons (2) move in the cylinder (1) in the same direction with the same speed, the so-called movement in a hydraulically and pneumatically neutral mode occurs, which does not generate fluid or gas pumping through the hoses (10,12,14,16) and allows the reaction solution to be transferred to the desired area of the cylinder (1), for example, to uptake of a specific reagent, exchange gas, carry out a determination or remove the solution from the cylinder (1) to the waste channel (61) by the hose (44).

The optical detection system (70), according to the invention, consists of a light source (71) and a pair of detectors (74,75) positioned at an angle of 90° to each other. This arrangement of detectors (74,75) enables photometric or turbidimeter detection using the detector (74) placed on the axis of the optical path (72) of the light source (71), on the opposite side of the cylinder (1), as well as carrying out fluorimetric or nephelometric detection using the detector (75) located on the axis of the optical path (73) perpendicular to the optical path (72), preferably crossing it on the axis of the cylinder (1). As the light source (71), for example, diodes, fluorescent lamps or light bulbs are used, oriented towards the interior of the cylinder (1). As detectors (74,75), for example, diodes, photodiodes, photoresistors, photomultipliers, CCD matrices or CMOS matrices are used, directed towards the interior of the cylinder (1). The light source (71) and detectors (74,75) can be brought to the desired location around the cylinder (1) via optical fibres. The width of the optical paths (72,73) is 1-10 mm, preferably 5 mm, which corresponds to the diameter of a standard diode and allows to use its maximum power. The width of the optical path is determined by the diameter of the hole in the light source housing or elements separating it from the cylinder (1). The axes of the optical paths (72,73) pass through the interior of the cylinder (1), preferably intersecting the axis of the cylinder (1), and also, the optical paths (72,73) preferably pass through the cylinder (1) with their entire width.

The optical detection system (70), according to the invention, may have different components depending on the application of the device. In the universal version, the optical detection system (70) has a light source (71) emitting light of an adjustable wavelength, preferably using a monochromator, and any pair of detectors (74,75), preferably in the form of diodes (FIG. 8). In another version, the optical detection system (70) has a light source (71) emitting white light with a continuous spectrum, and only one detector (74) in the form of a CMOS matrix (FIG. 9). When using a continuous-spectrum white light source (71), fluorimetric and nephelometric measurements are ineffective. In the device adapted to the determination of a specific analyte, the optical detection system (70) has a light source (71) emitting monochromatic light in the absorption or excitation range of the product of the specific reaction, and preferably, the light sources (71) and detectors (74,75) are optoelectronic elements, diodes and LED detectors, respectively. In the version for the determination of more than one specific analyte, the device comprises more than one optical detection system (70), and each of them has a light source (71) with different characteristics, adapted to the determination of a different product of a different specific reaction. It is possible to arrange optical detection systems (70) with appropriate diodes along the axis of the cylinder (1) (FIG. 10). Alternatively, a set of light sources (71) is created, for example three, which optical axes (72) intersect at one point, and three detectors (74) are located in their extension, and one common detector (75) is located on the axis of the optical path (73) at an angle of 90° to the axis of all the optical paths (72) (FIG. 11).

The above-described embodiment of the present invention can be characterised as follows:

1. A device for the automated determination of an analyte in the liquid phase by conducting a specific chemical reaction and subsequent optical measurement of the concentration of its products, containing a hydraulic system equipped with a set of hoses for pumping liquid solutions and reagents, a system for sampling the tested solution, a reservoir for collecting and storing of a reference portion of the matrix solution, a container for the analyte standard, containers for appropriately selected chemical reagents, and an optical detection system for determining the product of a specific reaction, where the containers for liquids are placed in a replaceable cartridge, which device has a reaction space in which a sampled portion of the tested solution or a reference sample or a standard solution with appropriately selected chemical reagents is placed, as well as a space for optical detection of the product of the specific reaction, and the device is electronically controlled by means of a main controller equipped with means for communication and transmission of information with electronic external devices, in which the device, according to the invention, the cylinder (1) acts as the reaction space and the optical detection space, with transparent walls in the range of the determination of the product of the specific reaction, which cylinder (1) is equipped with two opposing coaxial movable pistons (2) tightly closing it on each side, driven by electronically controlled stepper motors (3) equipped with lead screws with positioning nuts, which are connected to the pistons (2) by the connectors (4), setting the pistons (2) in linear motion inside the cylinder (1), which device has: a set of at least four hoses (99A,99B,99C,99D) tightly embedded in the holes (10A,10B,10C,10D) in the wall of the cylinder (1), supplying liquid substances from at least four reservoirs (95A,95B,95C,95D) directly to the interior of the cylinder (1), a hose (41) tightly embedded in the hole (12) in the wall of the cylinder (1), supplying the tested solution from the sample source (50,60,62) directly to the interior of the cylinder (1), at least one hose (47) tightly embedded in the hole (14) in the cylinder wall (1), used for gas transmission and pressure equalisation inside the cylinder (1), a hose (44) tightly embedded in the hole (16) in the cylinder wall (1), draining liquid substances to the waste channel (61) directly from the interior of the cylinder (1), and at least one optical detection system (70) equipped with a light source (71) and a detector (74), the components of which are located around the cylinder (1) so that the optical path (72) connecting the light source (71) and the detector (74) passed through the interior of the cylinder (1), where the holes (10,12,14,16) in the cylinder wall (1) are arranged along its axis so that the minimum distance between their projections on the axis of the cylinder (1) is 2 mm, and the fluid flow in the hydraulic system is controlled pneumatically by changing the relative position of the pistons (2), which generates changes in gas pressure in a specific parts of this system forcing fluid movement to balance these changes, and the device is equipped with an alarm system (80) for verbalising the messages regarding the determination process.

2. In the present embodiment of the invention, the cylinder (1) has an internal diameter in the range of 4-20 mm, preferably 8-16 mm, most preferably 12 mm, while the cylinder (1) is made of glass, quartz, polypropylene or PET, and the pistons (2) one-piece or two-piece with an appropriately selected outer diameter in the range of 4-20 mm, preferably 8-16 mm, most preferably 12 mm are made of glass, quartz, polypropylene, PET, PEEK or steel, in a configuration with or without a gasket, wherein a body of a commercially available syringe without the bottom with a needle seat acts as the cylinder (1), while are pistons of the same commercially available syringe act as the pistons (2).

The distance between the projections of the position of the holes (10,12,14,16) on the axis of the cylinder (1) is 2-10 mm, preferably 5 mm, and the diameter of the holes (10,12,14,16) is equal to the outer diameter of the hoses (95,41,47,44).

3. Preferably, the cylinder (1) is oriented horizontally, wherein the diameters of the holes (10) lie on a vertical plane passing through the axis of the cylinder (1) at the intersection with the side surface of the cylinder (1) located above its axis, and the diameters of the holes (12,14,16) lie at the intersection of this vertical plane with the side surface of the cylinder (1) below its axis, with the upper holes and the lower holes located alternately, with their preferred sequence in any direction: 10A, 12, 10B, 14, 10C, 16, 10D.

Alternatively, the cylinder (1) is vertically oriented, the cylinder having at least 5 holes (14) connected to the hoses (47) for gas transfer and pressure equalisation which alternate with the holes (10A,10B,10C,10D,12,16), preferably located on the opposite side of the cylinder (1) to the holes (14), with their preferred sequence from top to bottom: 12, 14, 10A, 14, 10B, 14, 10C, 14, 10D, 14, 16.

4. In the present device, the hoses (95,41,47,44) are made of a chemically inert material, preferably PTFE or FEP, and have an internal diameter in the range of 0.4-2.0 mm, preferably 0.8 mm, and their wall thickness is 0.1-0.8 mm, preferably 0.25 mm.

5. The sample source for stationary regime measurements is in the form of a classic automatic sample changer (62), where the outlet of the hose (41), or the outlet of the tube being its extension, is placed sequentially in successive vials filled with successive tested samples.

The sample source for flow regime measurements is in the form of a sampling system consisting of a rigid curved tube (42) of a corresponding size as the hose (41), being its extension, which is inserted into the sample stream moving through the channel (60), wherein the curved tube (42) is directed upstream of the liquid sample stream.

6. In this embodiment, the device comprises at least four reservoirs (95A,95B,95C,95D) to store liquid chemical reagents, with the outlet directed upwards, preferably in the shape of conical bottom vials, preferably commercially available eppendorf vials for centrifuges, with a volume of 10-50 ml, preferably 25 ml, with a height of 50-100 mm, preferably 78 mm, wherein the reservoirs (95) have a closure (96) at the top, preferably in the form of a cap or a plug, with a hole of a diameter of 4-20 mm, preferably 6-14 mm, preferably closed with a septum membrane, and a 1 mm hole for pressure equalisation, wherein the reagents are taken from the reservoirs (95) by the hoses (99) inserted from the top, where the reservoirs (95) with closures (96) are rigidly embedded in the round sockets (97) receiving the closures (96) from the bottom, where the inner diameter of the sockets (97) corresponds to the outer diameter of the closures (96).

7. The present device includes the tips (98) embedded from the bottom in the sockets (97) which, after puncturing the septum membranes in the closures (96) of the reservoirs (95), are located inside these reservoirs (95), which tips (98) have the shape of cylinders with a diameter of 4-8 mm, preferably 6 mm, decreasing downwards, with hoses (99) passing through the tips (98), the outlets of which is located above the lower edge of the tips (98), preferably 0-2 mm above this edge, and the inner diameter of the lower edge of the tips (98) is equal to the outer diameter of the hoses (99), each of the tips (98) being made of a conductive material, preferably carbon-doped polypropylene, and is connected to an electronic system that measures their impedance, which allows the determination the fluid level in each reservoirs (95) during operation of the device, while the tips (98) are rigidly mounted from the bottom in the centre of the round sockets (97) receiving the closures (96) of the reservoirs (95) placed in these sockets (97) from the bottom, where the inner diameter of the sockets (97) corresponds to the outer diameter of the closures (96), and the distance of the lower edge of the tips (98) from their bottom of the reservoirs (95), after their proper placement in the sockets (97), equals 0.5-2.0 mm, preferably 1.0 mm.

8. In this embodiment, the cartridge (90) is in the form of a container having a frame (91) and elements positioning the reservoirs (95), containing at least four reservoirs (95A,95B,95C,95D), and seat receiving the cartridge (90), equipped with the guides (93) receiving the frame (91) of the cartridge (90) and means (94) for immobilising the cartridge (90) in the seat, preferably in the form of a lift, a lock with a release mechanism, a magnet assembly, an electromagnet assembly or a lockable door.

The seat for the cartridge (90) its upper plane has a set of sockets (97A,97B,97C,97D) coaxial with the reservoirs (95A,95B,95C,95D) in the cartridge (90).

Preferably, the cartridge (90) consists of two interconnecting parts, the first of which (91) contains the frame, cylinder (1) with pistons (2), connectors (4), hoses (99), hose/hoses (47), sockets (97) and guides with a stabilising element that receives the second part (92) containing positioning elements of the reservoirs (95), wherein the parts (91,92) are detachably and unambiguously connected, ensuring the coaxiality of the reservoirs (95) and sockets (97), thanks to which the placement of the reservoirs (95) is carried out in a repeatable manner, while the hose (41) connecting the cylinder (1) with the sample source (50,60,62) is divided into two fragments, one of which (41A), tightly embedded in the hole (12), is located in the cartridge (90) and ends with a connector (41B), and the other (41D) starts with a connector (41C) compatible with the connector (41B), embedded in the bed receiving the cartridge (90), and further runs in the body of the device, connecting to the sample source (50,60,62), while the hose (44), connecting the cylinder (1) with the waste channel (61), is divided into two fragments, one of which (44A), tightly embedded in the hole (16), is located in the cartridge (90) and ends with a connector (44B), and the other (44D) starts with a connector (44C), compatible with the connector (44B), embedded in the bed receiving the cartridge (90), and continues in the body of the device connecting to the waste channel (61).

9. In this preferred embodiment, the optical detection system (70) consists of a light source (71), for example in the form of a diode, a fluorescent lamp or a light bulb, oriented towards the interior of the cylinder (1), and two detectors (74,75), for example in the form of a diode, photodiode, photoresistor, photomultiplier, CCD matrix or CMOS matrix, one of which (74), used in photometric or turbidimetric detection, is located on the axis of the optical path (72) if the light source (71) on the opposite side of the cylinder (1) facing towards the interior of the cylinder (1), while the other detector (75), used in fluorimetric or nephelometric detection, is located on the axis of the optical path (73) oriented at 90° to the axis of the optical path (72), facing towards the interior of the cylinder (1), wherein the light source (71) and the detectors (74,75) can be brought to the desired place via optical fibres, while the width of optical paths (72,73) equals 1-10 mm, preferably 5 mm, and the axes of the optical paths (72,73) pass through the interior of the cylinder (1), preferably, the axes of the optical paths (72,73) intersect with the axis of the cylinder (1), and the optical paths (72,73) preferably pass through the cylinder (1) with their entire width.

The optical detection system (70) has a light source (71) emitting light of an adjustable wavelength, preferably using a monochromator, or light source (71) emitting white light with a continuous spectrum, while having any pair of detectors (74,75), preferably in the form of diodes or the detector (74) is a CMOS matrix respectively.

Alternatively, the optical detection system (70) has a light source (71) emitting monochromatic light in the range of absorption or excitation of the product of the specific reaction, wherein the light source (71) and the detectors (72,73) are preferably optoelectronic elements, diodes and LED detectors, respectively.

Preferably, the present device has more than one optical detection system (70), and each of them has a light source (71) and a detector (74) with different characteristics, adapted to the determination of a different product of a different specific reaction, with the optical detection systems distributed along the axis of the cylinder (1) or concentrated in one area thereof, in a way where the optical axes (72,73) of the systems cross at one point.

10. In the special case, the version of the device for tracking the progress of blood dialysis by examining changes in the level of toxins in the stream of post-dialysis fluid, adapted to determine creatinine, urea and phosphate ions, the device has one optical detection system (70) containing a light source (71) emitting light of an adjustable wavelength or white light with a continuous spectrum, or has at least 3 optical detection systems (70) containing different light sources (71) emitting monochromatic light, and different detectors (74,75), where the first optical detection system (70) for the determination of the adduct of creatinine with picric acid comprises a 500-550 nm diode, preferably 525 nm, as a light source (71), a 525 nm diode as a detector (74) and a 625 nm diode as a detector (75), the second optical detection system (70) for the determination of urea adduct with 4-(dimethylamino) benzaldehyde, comprises a 410-460 nm diode, preferably 415 nm, as a light source (71) and a 460 nm diode as a detector (74) and a 625 nm diode as a detector (75), while the third optical detection system (70), for the determination of phosphoromolybdenum blue, comprises a 550-900 nm diode, preferably 625 nm, as a light source (71) and 625 nm diodes as a detector (74) and a detector (75).

11. According to the invention, the device is connected to the sample source (60) through an airlock (50), preferably the airlock (50) is located on the waste stream channel of the artificial kidney (60) above the sampling point with a hose (41).

12. According to the invention, the alarm system (80), equipped with a speaker (81), a light source (82) and means of remote communication (83), connected to the main controller (88) of the device, which automatically sends an appropriate message, for example after achieving the assumed analytical effect or in the event of deviation of the analytical result from the expectations in relation to a given measurement, automatically triggering a sound and a light signal on the device, and sending information about the achievement of the assumed analytical effect to the peripheral devices, preferably to the display on the device and to the operator's phone.

13. The above-described embodiment of the device, according to the present invention, is used in the method of automated determination of an analyte in the liquid phase by conducting a specific chemical reaction and subsequent optical measurement of the concentration of its products, which method uses the above device, whereby a specific chemical reaction is selected for a specific analyte, and then the wavelength is selected to determine the product of this specific reaction, and then the device is adapted to the given determination by selecting the appropriate light source (71) and the detector (74), the content of the cartridge (90) is adjusted by filling the reservoir (95A) with the analyte standard solution and reservoirs (95B,95C) with chemical reagents necessary to carry out the specific reaction, and then the cartridge (90) is placed in the dedicated bed in the device, after which a portion of the solution to be determined is sampled from the sample source (50,60,62) by dragging its portion through the hose (41) to the cylinder (1), and then portions of reagents are taken sequentially from the reservoirs (95B,95C) in the cartridge (90) to the cylinder (1), refilling or reducing the gas content in the cylinder (1) between the pistons (2) using the port (14) and the hose (47), wherein the volume of the reaction solution is preferably 428-1040 μl in the system with a horizontal cylinder (1), with the optical path width (72) of 5 mm and the distance between the projections of the holes (10A,12,10B,14,10C,16,10D) on its axis of 5 mm, or 565-1040 μl in a system with a vertical cylinder (1), with an optical path width (70) of 5 mm and a distance between the projections of the holes (12,14,10A,14,10B,14,10C,14,10D,14,16) on its axis of 5 mm, wherein the reaction solution is mixed by passing the gas through the reaction solution by drawing it into the cylinder (1) from the hole (14), and then the reaction solution is moved between the pistons (2) to the area of the appropriate detection system (70), ensuring the level of the liquid batch in the cylinder (1) enabling the passage of the optical path (72) through the solution to be determined, preferably the level completely covering the optical path (72), after which, after a certain time, the concentration of the specific reaction product is optically determined using the optical detection system (70) by photometric, turbidimetric, fluorimetric or nephelometric measurement, or a combination thereof, and then the post-reaction solution is pumped out form of the cylinder (1) to the waste channel (61), and then the cylinder (1) is cleaned by washing it with the matrix solution from the reservoir (95D) or with a fresh portion of the test solution from the sample source (50,60,62), which is then pumped out of the cylinder (1) to the waste channel (61), while the fluid flow in the hydraulic system is pneumatically generated by changing the relative position of the pistons (2) in the cylinder (1) when the hole (14) is located outside the reaction space between the pistons (2), and when it is necessary to refill or reduce the amount of gas between the pistons (2), without generating liquid flow in the system, the pistons (2) are moved when the hole (14) is located in the space between the pistons (2), while when it is necessary to move the reaction solution to the desired area of the cylinder (1), the pistons (2) are moved in a hydraulically and pneumatically neutral mode with the same speed and direction inside the cylinder (1), wherein when the readings are consistent with the assumed final analytical effect or they are indicating a deviation from the intended procedure, the alarm system (80) is triggered.

The matrix solution devoid of analyte is placed inside the reservoir (95D) in the cartridge (90) before placing the cartridge (90) in the device or the matrix solution is sampled from the sample source (50,60,62) by drawing a portion of it through the hose (41) to the cylinder (1), and then pumping this portion of matrix solution into the reservoir (95D) through the hole (10D) and the hose (99D) and the sampling and pumping process repeated until the desired level of the matrix solution is reached in reservoir (95D).

The measurement for the proper sample is preceded by calibration measurements using the standard solution from the reservoir (95A) and the matrix solution from the reservoir (95D).

The alarm system (80) equipped with a speaker (81), a light source (82) and means of remote communication (83) automatically sends a message about the achievement of the assumed analytical effect or about deviations of the analytical result from the expectations in relation to the given measurement, automatically activating the sound and light signal on the device, and sending the information about the achievement of the assumed analytical effect to the peripheral devices such as the display on the device or the operator's phone.

In the particular case, to track the progress of blood dialysis by examining changes in the level of toxins in the stream of post-dialysis fluid flowing out of the waste channel from the haemodialysis machine,
a device having one optical detection system (70) comprising a light source (71) emitting light with an adjustable wavelength or white radiation with a continuous spectrum, or
a device having at least 3 optical detection systems (70) comprising different light sources (71) emitting monochromatic light of a wavelength of 500-550 nm, preferably 525 nm, 410-460 nm, preferably 415 nm, and 550-900 nm, preferably 625 nm, with different detectors (74) of a wavelength of 525 nm, 460 nm and 625 nm, respectively, as well as identical detectors (75) of a wavelength of 625 nm,
where, prior to monitoring the progress of dialysis, the analyte (toxin) for determination is selected from creatinine, urea and phosphate ions, and then a standard solution is placed in the reservoir (95A), and chemical reagents are placed in the reservoirs (95B,95D) to carry out a specific reaction, respectively:
   when determining creatinine: an aqueous standard solution of creatinine [CAS 60-27-5], an aqueous solution of picric acid [CAS 88-89-1], and an aqueous solution of NaOH [CAS 1310-73-2],
   for the determination of urea: an aqueous standard solution of urea [CAS 54-13-6], an aqueous ethanolic solution of 4-(dimethylamine)benzaldehyde [CAS 100-10-7] and of hydrochloric acid [CAS 7647-01-0], and an aqueous solution of hydrochloric acid [CAS 7647-01-0],
   and for the determination of phosphate ions: an aqueous standard solution of phosphate ions, an aqueous solution containing ammonium orthomolybdate [CAS 236-031-3], potassium antimonyl tartrate [CAS 28300-74-5] and sulfuric acid [CAS 76664-93-9] and an aqueous solution of ascorbic acid [CAS: 50-81-7],
and then the cartridge (90) is placed in the dedicated bed in the device, the matrix solution being a pure dialysis fluid, which is placed in the reservoir (95D) before placing the cartridge (90) in the dedicated bed or prior to starting the actual dialysis the dialysis fluid is sampled from the waste stream (60) of the artificial kidney connected to the artificial kidney through the airlock (50), wherein an initial calibration measurement is carried out, and then the post-dialysis fluid is sampled from the waste stream (60) of the artificial kidney at regular intervals, for example every 5-15 minutes, and the temporary concentration of the analyte in the dialysate stream is determined, while the cylinder (1) is preferably washed between the successive samples and determinations of the analyte with the matrix solution from the reservoir (95D) or with a portion of the dialysate stream of the current composition, and preferably calibration measurements are conducted between the successive samples and determinations of the analyte using the standard solution from the reservoir (95A), wherein the dependence of the decrease of the toxins' content in the dialysate as a function of time is tracked on the electronic external device, and when the toxin level, successively decreasing, reaches the normative level that would be observed for a healthy person, indicating the patient's blood has been effectively purified, the alarm system (80) is activated informing about the possibility of termination of the dialysis, or when the toxin level behaves abnormally, the alarm system (80) is activated informing about possible errors in the dialysis process.
   Reaction-Detection System with Cartage According to the Second Embodiment (Variant II)

As in variant I, the cylinder (1) controls the flow of fluids in the hydraulic system of the device, and also constitutes the space for conducting the specific reaction and the detection space, which is located at the intersection of the cylinder (1) with the detection chamber (6), which is a round-section opening in the housing block (5), perpendicular to the cylinder (1), located in a way that the axes of the cylinder (1) and the detection chamber (6) intersect directly above the hole (14) and the channel (15). The cylinder (1) is still used as an independent element with transparent walls, which, unlike the solution in variant I, is stabilised by the housing block (5). In the case of using brittle structural material of the cylinder (1), such as glass or quartz, a gasket (9), preferably a two-piece gasket, is used between the cylinder (1) and the housing block (5), preferably flat, made of ethylene-propylene-diene monomer rubber (EPDM), silicone or polyurethane. The two-piece housing block (5) is clamped on the cylinder (1) in a configuration with a gasket (9) (FIG. 21, FIG. 25), using assembly screws passing through the assembled housing block (5), which ensures tightness of the connection of the holes (10,12,14,16) in the cylinder (1) with the channels (11,13,15,17) in the housing block (5). Alternatively, the cylinder (1) is made of a transparent polymeric material such as acrylic glass (PMMA), polystyrene (PS), polycarbonate (PC) or polypropylene (PP). Then, there is no need to use a gasket (9), but the cylinder (1) is permanently connected to the housing block (5) using an adhesive chemically inert to the reagents used, for example an acrylic or a silicone adhesive. Embedding the cylinder (1) in the housing block (5) ensures greater mechanical durability of the system, while ensuring satisfactory spectral transparency in the standard detection range, preferably in the range of visible light, near infrared and near ultraviolet region, which ensures the possibility of conducting of a variety of specific reactions. The housing block (5), produced by simple, classical machining techniques, has a circular cavity in which the cylinder (1) is embedded, optionally with a gasket (9).

The housing block (5) is made of a chemically inert, rigid material with good machinability characteristics, such as polyetheretherketone (PEEK), acrylic glass (PMMA), poly(acrylonitrile-co-butadiene-co-styrene) (ABS) or polyamide (PA). It is also possible to machine the housing block (5) from metal, for example stainless steel or aluminium, wherein aluminium is used only if alkaline reagents are not used. The pistons (2) have rods made of a chemically inert, rigid plastic material such as polyethylene terephthalate (PTFE), polyamide (PA), poly(acrylonitrile butadiene styrene) (ABS), polyether ether ketone (PEEK) or polypropylene (PP), and their guide holders are made of metal such as brass, aluminium or steel. The piston rods may be equipped with a gasket, preferably a flat gasket, made of ethylene propylene diene monomer rubber (EPDM), silicone or polyurethane. The cylinder (1) and the pistons (2) are precisely made so that the system remains tight. The cylinder (1) has an internal diameter in the range of 3.00-8.00 mm, preferably 4.00-7.00 mm, most preferably 6.00 mm. The pistons (2) can be single-piece or multi-piece (i.e. equipped with a gasket) and have an outer diameter matching to the size of the cylinder (1) ensuring tightness of the system and the possibility of its movement. The outer diameter of the pistons (2) is in the range of 3.00-8.20 mm, preferably 3.15-7.20 mm, most preferably 6.10 mm, i.e. the dimension of the pistons (2) is the same as the dimension of the cylinder (1) or is 0.05-0.15 mm, preferably 0.10 mm, larger than the dimension of the cylinder (1). The cylinder (1) has an outer diameter in the range of 4.00-14.00 mm, preferably 10.00 mm, i.e. the preferred wall thickness of the cylinder (1) is 2 mm. The gasket (9) is preferably 1 mm thick, which ensures that it is flexible enough to assemble the two-piece housing block (5). Preferably, two flat gaskets are used, preferably with a length corresponding to the length of the housing block (5) and a width not greater than half of the circumference of the cylinder (1), and they are applied to the cylinder (1) from above and below in such a way as to seal the connection area of the holes (10,12,14,16) in the cylinder (1) with the channels (11,13,15,17) in the housing block (5). Alternatively, a single gasket is used, preferably with a length corresponding to the length of the housing block (5) and a width greater than half the circumference of the cylinder (1), which is applied to the cylinder (1) in such a way as to seal the connection area of the holes (10,12,14,16) in the cylinder (1) with the channels (11,13,15,17) in the housing block (5). The one-piece gasket also has a through opening coaxial with the opening of the detection chamber (6), with dimensions not smaller than the opening of the detection chamber (6). The gasket (9) has holes coaxial with the holes (10,12,14,16), with an internal diameter equal to their diameter.

The cylinder (1) is equipped with two pistons (2), thanks to which it can be used not only to force the flow of the liquids in the hydraulic system, but also to fully control the stoichiometry of the reaction and the determination of its products without the need of using additional valves and other components to control the liquid flow. According to the invention, the cylinder (1) is directly connected through the holes (10) to reservoirs (20) in the inverted cartridge (30), which are used to store the standard solution (for example, reservoir 20A), the reagents (for example, reservoirs 20B and 20C) and to mix the working solution (for example, reservoir 20D). In addition, the cylinder (1) is directly connected through the hole (12) to the sample source in the form of an automatic sampling system or sample changer (62) or a pipe (60) through which the stream of the tested (monitored) sample flows, or an airlock (50) on the pipe (60). The use of an airlock (50) is particularly advantageous as it allows to avoids the risk of contamination of the tested sample stream. An airlock (50) is preferably used. The cylinder (1) is also connected via the holes (14,16) to the waste channel (61) for removing waste fluids from the reaction space. The drain using the hole (14) is used to remove the reaction solution after detection without having to move it to another area of the cylinder (1), while the drain using the hole (16) is used to quickly remove the residual reaction solution from the mixer (20D). In the current solution, there is no need to uptake gas from the outside in order to equalise the pressure inside the hydraulic system.

The connection of the cylinder (1) through the holes (10) with the reservoirs (20) in the cartridge (30) is carried out by channels (11) in the housing block (5), ports (28), with a stepped undercut (29) and side-sealing gaskets (27) at the housing block (5), cooperating with the movable lid (26), receiving through pins (25) at the outlet of the channels (24), leading to sockets (23) of the dispensing tips (22) of the reservoirs (20) in the cartridge (30). The connection of the cylinder (1) to the sample source (50,60,62) is carried out through the hole (12) and the channel (13), and its connection to the waste channel (61) is carried out through the holes (14,16) and the channels (15,17). The channels (13,15,17) are equipped with quick connect fittings (40,45,43) at their outlet, respectively, receiving the hoses (41,46,44). Preferably, the quick connect fittings (40,45,43) are of the FESTO/SMC type, mounted in threaded holes in the housing block (5), which end the channels (13,15,17) on the outer surface of the housing block (5), preferably on the bottom surface, preferably on the tips sticking beyond the housing block (5). Hoses (41,44,46) are made of a flexible, rigid, chemically inert material, for example perfluorinated polymers, preferably poly(tetrafluoroethylene) (PTFE), fluorinated ethylene propylene (FEP) or NAFION (copolymer of tetrafluoroethylene and sulfone-terminated perfluorinated oligovinyl ether).

The housing block (5) has external dimensions ensuring the stability of the reaction area, i.e. stiffness, dimensional stability and tightness of the cylinder (1) with the pistons (2). The internal dimensions of the cylinder (1) ensure freedom of movement of the pistons (2) with the connectors (4). The housing block (5) is preferably in the form of a cuboid 85-105 mm long, 25-40 mm wide and 25-80 mm high, preferably the housing block (5) has dimensions of 94×28×33 mm. It is possible to use the housing block (5) in a different shape, but this unnecessarily complicates the production process and the way of mutual arrangement of the functional elements of the device inside its housing. The housing block (5) has a circular cavity with a diameter of 5-15 mm, preferably 10 mm or 12 mm, respectively for the cylinder (1) or the cylinder (1) with the seal (9). From this opening, through channels (11,13,15,17) extend towards reservoirs (20) in the cartridge (30), the sample source (50,60,62), and the waste channel (61), respectively. The channels (11,13,15,17) have a diameter in the range of 0.8-2.0 mm, preferably 1.0 mm, and are preferably perpendicular to the axis of the cylinder (1).

The detection chamber (6) is a through opening in the housing block (5), preferably with a circular cross-section, perpendicular to the axis of the cylinder (1). Such arrangement of the detection chamber (6) allows the use of an optical detection system (70) with one detector (74) located on the axis of the optical path (72) of the light source (71), which allows for photometric or turbidimetric detection. The detection chamber (6) has an internal diameter in the range of 3-10 mm, preferably 4-6 mm, most preferably 4 mm. The detection chamber (6) has no windows (8), because the reaction solution is tested directly inside the cylinder (1) with the walls transparent in the range of the determination of the product of the specific reaction. Directly below the detection chamber (6) there is a channel (15) that connects the cylinder (1) to the waste channel (61).

The diameter of the holes (10,12,14,16) in the cylinder wall (1) is equal to the diameter of the channels (11,13,15,17) coaxial with them in the housing block (5). Preferably, the diameter of these holes is 0.8-2 mm, preferably 1.0 mm. Preferably, all openings (10,12,14,16), channels (11,13,15,17), passage elements of other parts of the device (24,25) and hoses (41,44,46) have the same inner diameter, in the range of 0.8-2.0 mm, preferably 1.0 mm.

The holes (10,12,14,16) are located in different parts of the cylinder (1) in order to ensure that the fluid can be transferred to each of the channels (11,13,15,17), coaxial with them, separately. The distance between the projections of the axes of the holes (10,12,14,16) on the axis of the cylinder (1) is 10-14 mm, preferably 11 mm. The spacing of the holes (10,12,14,16) at a distance of 11 mm is advantageous due to the optimisation of the working volume of the cylinder (1) while ensuring its relatively small beneficial diameter of 6 mm. Using 11 mm spacing of the holes (10,12,14,16) with a preferred internal diameter of 1.0 mm, and the hoses supplying the sample (41) and draining the post-reaction mixture (46) and waste solutions (44) with a preferred internal diameter of 1 mm and a preferred external diameter of 4 mm, the maximum volume of the working space between the pistons (2), ensuring contact with a single hole (10,12,14,16) in the cylinder (1) with a diameter of 6 mm is 650 μl, which corresponds to the opening of the pistons (2) by 23 mm, and the maximum working volume for moving the pistons in hydraulically and pneumatically neutral conditions is 420 μl, which corresponds to the opening of the pistons (2) by 15 mm, i.e. such opening ensuring exposure to a single hole (10,12,14,16) in each position of the pistons (2) spaced in this way. By using one of the reservoirs (20D) in the cartridge (30) as a place for storing and mixing the working solution, it is possible to carry out determinations using a working solution with a maximum volume of 10,000 μl, i.e. the nominal volume of the reservoir (20D) in the cartridge (30), however, this volume is unfavourable due to the limited possibility of mixing solutions in the reservoir. The practical maximum volume is preferably around 1000-1200 μl. To ensure adequate working space inside the cylinder (1), its length is 85-105 mm, preferably 94 mm. The use of a cylinder (1) of a favourable length has the advantage that it allows the use of commercially available lead screws of the stepper motors (3) with a standard length of 200 mm, without the need to modify them, which greatly simplifies the production process and reduces its costs.

The cylinder (1) can be placed in the device at any angle in relation to the surface, but the variant of horizontal orientation is considered to be particularly advantageous.

The holes (10,12,14,16) of the cylinder (1) are coaxial with the channels (11,13,15,17) in the housing block (5), with the holes and the channels in pairs (10-11, 12-13, 14-15, 16-17) have the same diameter. The housing block (5) is machined mechanically, preferably with classical techniques, by drilling a cavity for the cylinder (1) and the opening for the detection chamber (6), connecting the housing block (5) with the cylinder (1) and then drilling the channels (11,13,15,17) with appropriately designed outer outlets, i.e. ports (28) with stepped undercuts (29) at the outlet of the channels (11) and threaded holes accepting quick connect fittings (40,43,45) at the outlet of the channels (13,17,15). It is possible to produce these elements using other techniques, for example additive techniques, but lossy machining (cutting, turning, hollowing, etc.) is the cheapest and fastest while maintaining the desired precision and quality of finish. A finished functional element is obtained this way.

The reaction-detection system according to the invention, in variant II, has a replaceable inverse cartridge (30) containing at least four reservoirs (20A,20B,20C,20D) for chemical reagents and liquids necessary for carrying out the determinations. Construction as well as technical and analytical functions of the cartridge (30) in variant II are the same as in the preferred variant Ill of the reaction-detection system according to the invention, and are described below in the detailed description of variant III.

The optical detection system (70), according to the invention, has the same mode of operation and similar characteristics as in the variant I described above. The differences are subtle and result from the differences in the design of the detection chamber (6), as well as from the use of a CCD matrix with RGB filters. The elements of the optical detection system (70) are located around the detection chamber (6), oriented towards the interior thereof. The optical path (72), connecting the light source (71) and the detector (74), passes with its entire width through the interior of the detection chamber (6), preferably through the intersection of the axis of the opening constituting the detection chamber (6) with the axis of the cylinder (1) and the channel (15). In contrary to the previous solution, due to design limitations and manufacturing convenience, when drilling the detection chamber (6) perpendicular to the front plane of the housing block (5), the optical detection system (70) preferably has one detector (74), because the second detector (75) in the optical path (73), perpendicular to the optical path (72), would have to be located in the vertical axis of the detection block, where it would interfere with the arrangement of the ports (28) or the arrangement of the quick connect fittings (40,43,45). Nevertheless, it is possible to make an optical detection system (70) with two detectors, but this requires drilling through openings transverse to the cylinder (1), with axes crossing the axis of the cylinder (1), to create the detection chamber (6), at an angle of 45° to the axis of the channel (15), and at the same time at an angle of 45° to the front plane of the housing block (5), and also to create auxiliary planes at the outlets of these openings, necessary for mounting the light source (71) and the detectors (74,75), which is burdensome and significantly increases production costs (FIG. 31). The use of an optical detection system (70) with a single detector (74) is therefore preferred, especially in the preferred system with an integrated SMD LED as an emitter (71) and a CCD matrix with an RGB filter as a detector (74), which provides adequate measurement quality for the determination of the product of the specific reaction, according to the invention.

The optical detection system (70) consists of a light source (71), for example in the form of a diode, a fluorescent lamp or a light bulb, oriented towards the interior of the detection chamber (6), and a detector (74), for example in the form of a diode, a photodiode, a photoresistor, a photomultiplier tube, a CCD matrix or a CMOS matrix. A detector (74), for photometric or turbidimetric detection, oriented towards the interior of the detection chamber (6) is located on the axis of the optical path (72) of the light source (71) on the opposite side of the detection chamber (6). The light source (71) and the detector (74) are preferably mounted directly around the detection chamber (6), but can also be brought to the desired location via optical fibres. The optical path width (72) is 1-10 mm, preferably 4 mm, and the axis of the optical path (72) passes through the interior of the detection chamber (6), preferably together with the entire optical path width. The present invention provides four forms of the construction of the optical detection system (70) for variant II described herein.

The optical detection system (70) has a light source (71) emitting light of an adjustable wavelength, preferably equipped with a monochromator, while the detector (74) is a CCD matrix (FIG. 26). Alternatively, the optical detection system (70) has a light source (71) emitting white light with a continuous spectrum, and the detector (74) is a CMOS matrix (FIG. 27). Both of these forms are equipped with a universal light source and universal detectors, thanks to which they can be used to determine the product of any specific reaction. However, these systems are expensive and complicated, which may exclude the possibility of their routine use for repeated determinations of one analyte.

Preferably, the optical detection system (70) has a light source (71) emitting monochromatic light in the range of absorption or excitation of the product pf the specific reaction, preferably an LED diode, while the detector (74) is a CCD matrix, preferably with an RGB filter, or a specific LED detector with characteristics adapted to the specific analyte (FIG. 28). Alternatively, the optical detection system (70) has a light source (71) emitting monochromatic light of several wavelengths in the range of absorption or excitation of the products of the specific reactions, preferably an integrated SMD diode equipped with several LEDs, wherein the optical axes (72) of these diodes pass through the axis of the cylinder (1), while the detector (74) is a CCD matrix, preferably with an RGB filter, or several specific LED detectors with characteristics adapted to the specific analytes (FIG. 28). Both of these forms have a light source (71) adapted to the determination of a specific analyte and universal detectors, thanks to which they can be used only to determine the product of a specific reaction. This functionality is suitable for routine use with repeated determinations of a single analyte.

Most preferably, the optical detection system (70) has a light source (71) emitting monochromatic light of several wavelengths in the range of absorption or excitation of the product of the specific reactions, preferably the light source (71) is an integrated SMD LED equipped with several LEDs, with the optical axes (72) of these diodes passing through the axis of the cylinder (1), while the detector (74) is a CCD matrix, preferably with an RGB filter, with characteristics adapted to the specific analytes (FIG. 28). This solution ensures the functional flexibility necessary for routine determinations of selected analytes and very good quality of the obtained data with the simplicity of construction and low production costs.

The above-described embodiment of the present invention can be characterised as follows:

1. A reaction-detection system with a replaceable cartridge for a device for automated determination of an analyte in the liquid phase by conducting a specific chemical reaction and subsequent optical measurement of the concentration of its products, in particular for monitoring the progress of the dialysis process, constituting an element of a hydraulic system equipped with a network of channels for pneumatic pumping of liquid solutions, wherein a single cylinder acts as a reaction space and an optical detection space, which cylinder has walls transparent in the range of determination of the product of the specific reaction, which cylinder is equipped with two opposing coaxial movable pistons closing it on each side, moved by electronically controlled stepper motors equipped with lead screws with positioning nuts, which are connected to the pistons by dedicated connectors, making the pistons to move linearly inside the cylinder, which cylinder is directly connected by separate channels/hoses to at least four reagent reservoirs in a replaceable cartridge, a sample source and a waste channel, and the determination of the analyte is carried out in an optical detection system for the determination of the product of the specific reaction, consisting of from a detection chamber equipped with transparent walls for the determination of specific reaction products, a light source and a detector/detectors for photometric, turbidimetric, fluorimetric or nephelometric detection, which is located on the axis of the optical path of the light source facing the interior of the detection chamber, where the light source and the detector are placed around the detection chamber, and the optical path connecting the light source and the detector passes through its interior, in which arrangement, according to the invention, the cylinder (1), with two pistons (2) moved by stepper motors (3) connected to them by connectors (4), is tightly embedded inside the housing block (5) and tightly connected to it by its outer surface, which cylinder (1) has holes (10,12,14,16) connected to the channels (11,13,15,17) in the housing block (5), where these openings and channels in pairs (10-11, 12-13, 14-15, 16-17) have the same diameter, and each of at least four holes (10) and corresponding channels (11) is detachably connected to one reservoir (20) in the cartridge (30) through ports (28) with stepped undercuts (29), equipped with side-sealing gaskets (27) and a pressing lid (26), receiving detachably through pins (25) of the cartridge (30), connected by channels (24) with the sockets (23) detachably accepting the dispensing tips (22) of the reservoirs (20), while the hole (12) and the channel (13) equipped with a quick connect fitting (40) are detachably connected by the hose (41) to the sample source, i.e. with the automatic sampling system (62), the pipe (60) with the sample stream or the airlock (50) on the pipe (60), while the holes (14,16) and channels (15,17) equipped with quick connect fittings (45,43) are detachably connected by the hoses (46,44) to the waste channel (61), whereby the hose (46) can be advantageously connected to the hose (44) earlier, and moreover, the housing block (5) has at least one transverse through opening with a circular cross-section, constituting the detection chamber (6), revealing the transparent walls of the cylinder (1), allowing for the assembly of elements of the optical detector system (70) on its both sides, wherein the detection block (6) and the cylinder (1) are perpendicular to each other and their axes intersect, preferably directly above the inner outlet of the hole (14) and the channel (15).

2. In this embodiment of the reaction-detection system with a replaceable cartridge, the cylinder (1) has an internal diameter in the range of 3-8 mm, preferably 4-7 mm, most preferably 6 mm, thickness in the range of 0.25-3.00 mm, preferably 1.00-2.50 mm, most preferably 2.00 mm, outer diameter in the range of 3.50-14.00 mm, preferably 10.00 mm, length in the range of 85-105 mm, preferably 94 mm, the cylinder (1) is made of glass, quartz, acrylic (PMMA), polystyrene (PS), polycarbonate (PC), polyethylene terephthalate (PET) or polypropylene (PP), and its pistons (2), with an appropriately selected outer diameter in the range of 3.2-8.2 mm, preferably 4.2-7.2 mm, most preferably 6.1 mm, tightly embedded in the cavity of the cylinder (1), with the piston rods made of chemically inert rigid plastic material, such as poly(ethylene terephthalate) (PTFE), polyetheretherketone (PEEK), poly(acrylonitrile-co-butadiene-co-styrene) (ABS), polyamide (PA), or polypropylene (PP), optionally with a gasket, preferably flat, made of ethylene-propylene-diene monomer rubber (EPDM), silicone or polyurethane, and guide holders made of metal such as brass, aluminium or steel.

3. Preferably, the cylinder (1) is made of glass or quartz and is additionally equipped with at least one gasket (9), preferably flat, made of silicone, polyurethane or ethylene-propylene-diene monomer rubber (EPDM), preferably 1 mm thick, preferably with two gaskets (9), with through holes coaxial with the holes (10,12,14,16) in the wall of the cylinder (1), with an internal diameter equal to thereof, sealing the joints of the holes (10,12,14,16) with the corresponding channels (11,13,15,17) in the housing block (5), and the two-piece housing block (5) is clamped on the cylinder (1) with a gasket (9) by means of screws passing through the vertical through mounting slots passing coaxially through both elements of the housing block (5), or alternatively, the cylinder (1) is made of acrylic glass (PMMA), polystyrene (PS), polycarbonate (PC), polyethylene terephthalate (PET) or polypropylene (PP), is permanently connected to the housing block (5) by means of an adhesive chemically inert to the reagents used, for example an acrylic or a silicone adhesive.

Preferably, the cylinder (1) is oriented horizontally, wherein the holes (10A,10B,10C,10D) lie at the intersection of the vertical plane passing through the axis of the cylinder (1) with the side surface of the cylinder (1) located above its axis, and the holes (12,14,16) are located at the intersection of this plane with the side surface of the cylinder (1) below its axis, where the upper holes (10) and the lower holes (12,14,16) are spaced alternately, with their favourable sequence in any direction: 10A, 12, 10B, 14, 10C, 16, 10D, and the distance between the projections of their axes on the axis of the cylinder (1) equals 10-14 mm, preferably 11 mm, and the diameter of the holes (10,12,14,16) equals 0.8-2.0 mm, preferably 1 mm.

4. According to the invention, the housing block (5) has the form of a cuboid with a length of 85-105 mm, a width of 25-40 mm and a height of 25-80 mm, optionally one-piece or two-piece cut in a horizontal plane containing the axis of the cylinder (1), preferably the housing block (5) has dimensions of 94×28×33 mm, which housing block (5) has through channels (11) connecting the holes (10) of the cylinder (1) with the ports (28), through channels (13,15,17) connecting the holes (12,14,16) of the cylinder (1) with quick connect fittings (40,45,43), wherein the channels (11,13,15,17) have diameters in the range of 0.8-2.0 mm, preferably 1 mm, and are preferably perpendicular to the axis of the cylinder (1), and the detection chamber (6) has an internal diameter in the range of 3-10 mm, preferably 4-6 mm, most preferably 4 mm, where the housing block (5) is made of a rigid chemically inert material, preferably polyetheretherketone (PEEK), acrylic glass (PMMA), poly(acrylonitrile-co-butadiene-co-styrene) (ABS), polyamide (PA), aluminium or stainless steel.

The detection chamber (6) is formed by two perpendicular, through openings, perpendicular to the cylinder (1), preferably oriented at an angle of 45° to the axis of the channel (15), preferably with a circular cross-section, with an internal diameter in the range of 3-10 mm, preferably 4-6 mm, most preferably 4 mm.

5. In the current reaction-detection system with a replaceable cartridge, the hoses (41,44,46) are made of a chemically inert material, preferably poly(tetrafluoroethylene) (PTFE), fluorinated ethylene-propylene (FEP) or NAFION (a copolymer of tetrafluoroethylene and perfluorinated oligovinyl ether terminated with a sulfone group), and have an internal diameter in the range of 0.8-2.0 mm, preferably 1.0 mm, and the thickness of their walls is 0.5-2.0 mm, preferably 1.5 mm, and are connected to the outlets of the channels (13,17,15) with quick connect fittings (40,43,45), preferably with FESTO/SMC type quick connect fittings with a flat gasket made of an elastic chemically inert material, preferably ethylene-propylene-diene monomers rubber (EPDM), silicone or polyurethane (PU), mounted in threaded holes ending the channels (13,17,15).

6. The sample source is a classic sampling system (62) in the form of an automatic sample changer, where the tip (42) of the hose (41) or its extension, at the time of sampling, is placed in a vessel filled with the tested sample, or the sample source is a pipe (60) with the sample stream, in which the rigid tip (42) of the hose (41) is placed with the opening directed upwards of the sample stream, or the sample source is an airlock (50) through which the sample stream flows brought by the pipe (60) with the sample stream, preferably the sample is taken from the accumulation reservoir (52) of the airlock (50) or its waste channel, and the end (42) of the hose (41) is directed with the opening downwards or upwards, respectively.

In the special case, when monitoring the progress of the dialysis process, the sample is taken from the accumulation reservoir (52) of the airlock (50) on the pipe (60) with the dialysate stream flowing directly from the haemodialysis machine.

7. In this embodiment, the cartridge (30) has at least four reservoirs (20A,20B,20C,20D), preferably in the form of syringes with pistons (21), made of chemically inert materials, preferably polypropylene (PP), preferably with a valid medical device certificate, with a volume in the range of 5-12 ml, preferably 10 ml, with dispensing tips (22), preferably of the LUER type, preferably located centrally in the axis of the reservoirs (20), seated tightly in a detachable manner with the outlet downwards in sockets (23), preferably of the LUER or LUER LOCK type, at the bottom of the housing (31) of the cartridge (30).

8. In addition, the cartridge (30) has the form of a container consisting of at least a housing (31), a lid (32) and a lock (33), preferably disposable, wherein the housing (31) of the cartridge (30) has a socket (23) at the inner bottom, preferably four sockets (23A,23B,23C,23D), preferably of the LUER or LUER LOCK type, each receiving one dispensing tip (22), preferably of the LUER type, of the reservoirs (20A,20B,20C,20D), which sockets (23) are connected by channels (24), preferably with a diameter equal to the diameter of the holes (10) and channels (11), equipped at the outlet on the bottom outer surface of the housing (31) of the cartridge (30) with through pins (25) with the outer diameter of 2.5-8 mm, preferably 6 mm, and a height of 5-20 mm, preferably 16 mm, compatible with ports (28) with an internal diameter of 2.7-8.2 mm, preferably 6.2 mm, and depth 6.2-21.2 mm, preferably 17.2 mm, where the depth of the stepped undercut (29) equals 4-6 mm, preferably 5 mm, and its diameter equals 6.25-14.25 mm, preferably 10.25 mm, having inside the side-sealing gaskets (27), preferably in the shape of an O-RING torus, with a diameter D/d of 4/1-8/3 mm, preferably 6/2 mm, and the lid (26) at the outlet of the channels (11) in the outer top surface of the housing block (5) of the cylinder (1), wherein the lid (26) is 0.5-2.0 mm higher, preferably 0.75 mm higher, than the difference in the height of the pin (25) and the thickness of the gasket (27), and one plane of the lid (26) rests against the bottom surface of the housing (31) of the cartridge (30), while the opposite plane of the lid (26) presses a gasket (27) when docking the cartridge (30), sealing the connection of the pin (25) with the port (28), while the housing (31,32) of the cartridge (30) additionally has side sockets (34) for the forks (35) of the lift (36), made of one bent metal element four-point fixed on the lift (36), which forks (35) have the shape of a horizontal cuboid ended with a trapezoidal tip stabilising the front of the cartridge (30), and also, the cartridge (30) has vertical through holes (37) for the posts (38) positioning the cartridge (30) in relation to the housing block (5), ensuring automatic movement, repeatable vertical movement of the cartridge (30) mounted on the forks (35) of the lift (36), as well as the correctness and repeatability of docking of the through pins (25) of the cartridge (30) in the ports (28) of the housing block (5) of the cylinder (1), and the construction material of the cartridge (30) is a thermoplastic, preferably polycarbonate (PC), polystyrene (PS), polyamide (PA) or poly(acrylonitrile-butadiene-co-styrene) (ABS).

In addition, the cartridge (30) has an electronic system (39) equipped with a non-volatile memory (NFC RFID TAG), wirelessly connected to the antenna of the electronic main controller (88) of the device when the cartridge (30) docked in the device, which memory is recognised by the electronic main controller (88), allowing the given cartridge (30) to be used once.

9. In this embodiment, the optical detection system (70) consists of a light source (71), for example in the form of a diode, a fluorescent lamp or a light bulb, oriented towards the interior of the detection chamber (6), and optionally one detector (74) or two detectors (74,75), for example in the form of a diode, photodiode, photoresistor, photomultiplier, CCD or CMOS matrix, one of which (74), for photometric or turbidimetric detection, oriented towards the interior of the detection chamber (6), is located on the axis of the optical path (72) of the light source (71) on the opposite side of the detection chamber (6), and the other (75), for fluorimetric or nephelometric detection, oriented towards the interior of the detection chamber (6), is located on the axis of the optical path (73) intersecting at an angle of 90° with the optical path (72) of the light source (71), wherein the light source (71) and the detectors (74,75) can be delivered to the desired location via optical fibres, wherein the width of the optical paths (72,73) equals 1-10 mm, preferably 4 mm, and the axes of the optical paths (72,73) pass through the interior of the cylinder (1), preferably with their entire width, and preferably cross at the point of geometric intersection of the axis of the opening/openings forming the detection chamber (6) with the axis of the cylinder (1) and the axis of the channel (15).

Furthermore, the optical detection system (70) has a light source (71) emitting light of an adjustable wavelength, preferably equipped with a monochromator, while the detector (74) is a CCD matrix and the detector (75) is a CCD matrix or a universal fluorimetric LED detector, or the optical detection system (70) has a light source (71) emitting white light with a continuous spectrum, while the detector (74) is a CMOS matrix and the detector (75) is a CMOS matrix or a universal fluorimetric LED detector, or the optical detection system (70) has a light source (71) emitting monochromatic light in the range of absorption or excitation of the product of the specific reaction, preferably an LED, while the detector (74) is a CCD matrix, preferably with an RGB filter, or a specific LED detector with characteristics adapted to a specific analyte, and the detector (75) is a CCD matrix, preferably with an RGB filter, or a universal fluorimetric LED detector, or an optical detection system (70) has a light source (71) emitting monochromatic light of several wavelengths in the range of absorption or excitation of the product of the specific reaction, preferably an integrated SMD LED equipped with several LEDs, where the optical axes (72) of these LEDs are parallel and lie in the axis of the SMD LED cover, coinciding with the axis of the opening constituting the detection chamber (6), while the detector (74) is a CCD matrix, preferably with an RGB filter, or several specific LED detectors with characteristics adapted to the specific analytes, and the detector (75) is a CCD matrix, preferably with an RGB filter, or a universal fluorimetric LED detector.

In a special case, the optical detection system (70) for monitoring the progress of the dialysis process, adapted for the determination of creatinine, urea and phosphate ions, has an integrated light source (71) in the form of a SMD LED diode emitting monochromatic light in the range of 500-550 nm, preferably 525 nm, for the determination of adduct of creatinine with picric acid, in the range of 410-460 nm, preferably 415 nm, for the determination of add urea with 4-(dimethylamino)benzaldehyde, and in the range of 550-900 nm, preferably 625 nm, for the determination of phosphomolybdenum blue, and has universal detectors (74,75) in the form of CCD matrices with RGB filters and channels operating in the red, green and blue light ranges.

10. This example of implementation of the present invention in the field of the reaction-detection system with a replaceable cartridge is used in the method of automated determination of an analyte in the liquid phase by conducting a specific chemical reaction and subsequent optical measurement of the concentration of its products, using the device for automated determination of the analyte in the liquid phase by conducting a specific chemical reaction and subsequent optical measurement of the concentration of its products, especially for monitoring the progress of the dialysis process, in which a specific chemical reaction tailored to a specific analyte and the wavelength for the determination of the product of the specific reaction is selected, then the device is adapted to the selected determination by adjusting the optical detection system, and the content of the cartridge is adjusted by filling the first reservoir with the analyte standard solution and the next two reservoirs with chemical reagents necessary to carry out the specific reaction, and then the cartridge is placed in the device, after which a portion of the solution to be determined is sampled by dragging its portion through the hose to the cylinder, and then portions of chemical reagents are sequentially taken from the two reservoirs of the cartridge, into the cylinder, the reaction solution is mixed, and then the reaction solution is transferred to the area of the optical detection system, ensuring the level of liquid that allows the optical path of the light source to pass through the solution to be determined, preferably the level of the solution completely covering the optical path, after which, after a certain time, the concentration of the specific reaction product is optically determined using an optical detection system by photometric, turbidimetric, fluorimetric or nephelometric measurement, or their combination, after which the post-reaction solution is pumped out to the waste channel, and then the cylinder is cleaned by washing it with a fresh portion of the tested solution drawn into the cylinder through the sampling hose, which is then pumped out of the cylinder into the waste channel, with the fluid flow in the hydraulic system being pneumatically generated by changing the relative mutual position of the pistons in in the cylinder, and in cases where it is necessary to move the reaction solution to the desired area of the cylinder, the pistons are moved in a hydraulically and pneumatically neutral mode with the same speed, direction and return inside the cylinder, while monitoring the dialysis progress, when the analyte concentration readings indicate a deviation from the expectations for a given analyte, or when the assumed analytical effect is achieved, the alarm system is activated, in which method, according to the invention, the device comprises a reaction and detection system with a replaceable cartridge described above, wherein the fourth reservoir (20D) in the cartridge (30) serves as a mixer, and all solutions drawn sequentially into the cylinder (1) during the determination of the test sample, i.e. a sample from the sample source (60,62,50), a matrix solution or a standard solution from the reservoir (20A) and reagents from the reservoirs (20B,20C) are pumped into the mixer immediately after being drawn into the cylinder (1) (20D) and after the uptake and transfer of all the solutions to the fourth reservoir (20D), the resulting reaction solution is mixed by pumping it between the cylinder (1) and the reservoir (20D), wherein the volume of the tested sample equals 30-90 μl, the volume of the reagents used equals 50-250 μl, which gives the reaction mixture of a volume of 240-320 μl, and when the mixing of the reaction solution is completed, its portion, preferably 240 μl, is pumped from the mixer (20D) to the cylinder (1), and then it is moved between the pistons (2) to the area of the detection chamber (6), where the analyte is determined, and after the determination, the reaction solution is pumped out from the cylinder (1) in the area of the detection chamber (6) and the reservoir (20D) through the channel (15) and a channel (17) to a waste channel (61), respectively.

Reaction-Detection System with Cartage According to the Third Embodiment (Variant II)

As in variants I and II described above, according to the invention, the cylinder (1) controls the flow of fluids in the hydraulic system of the device, and also constitutes a space for conducting a specific reaction. However, unlike variants I and II, the cylinder (1), due to the simplification of the design of the system, no longer constitutes the detection space, which has been moved to the detection chamber (6) in the detection block (7). This allowed to avoid the need to produce a cylinder (1) with transparent walls. In contrary to variants I and II, the cylinder (1) is not a separate element made of glass, quartz or other transparent material, but it is a through opening with a circular cross-section in the housing block (5), manufactured by simple, classic machining techniques as a through opening with a circular cross-section in the housing block (5).

The housing block (5) is made of a chemically inert, rigid material with good machinability characteristics, such as polyetheretherketone (PEEK), acrylic glass (PMMA), poly(acrylonitrile-co-butadiene-co-styrene) (ABS) or polyamide (PA). It is also possible to manufacture the housing block (5) from metal, for example stainless steel or aluminium, wherein aluminium is used only if alkaline reagents are not to be used. The pistons (2) have rods made of a chemically inert, rigid plastic material such as polyethylene terephthalate (PTFE), polyamide (PA), poly(acrylonitrile butadiene styrene) (ABS), polyetheretherketone (PEEK) or polypropylene (PP), and their guide holders are made of metal such as brass, aluminium or steel. The piston rods may be equipped with a gasket, preferably a flat gasket, made of ethylene propylene diene monomer rubber (EPDM), silicone or polyurethane. The cylinder (1) and the pistons (2) are precisely made so that the system remains tightness. The cylinder (1) has an internal diameter in the range of 3.00-8.00 mm, preferably 4.00-7.00 mm, most preferably 6.00 mm. The pistons (2) can be single-piece or multi-piece (i.e. equipped with a gasket) and have an outer diameter matching to the size of the cylinder (1) ensuring tightness of the system and the possibility of their movement. The outer diameter of the pistons (2) is in the range of 3.00-8.20 mm, preferably 3.15-7.20 mm, most preferably 6.10 mm, i.e. the dimension of the pistons (2) is the same as the dimension of the cylinder (1) or is 0.05-0.15 mm, preferably 0.10 mm, larger than the dimension of the cylinder (1).

The cylinder (1) is equipped with two pistons (2), thanks to which it can be used not only to force the flow of the liquids in the hydraulic system, but also to fully control the stoichiometry of the reaction and the determination of its products without the need of using additional valves and other components to control the liquid flow. According to the invention, the cylinder (1) is directly connected through the holes (10) to reservoirs (20) in the inverted cartridge (30), which are used to store the standard solution (for example, reservoir 20A), the reagents (for example, reservoirs 20B and 20C) and to mix the working solution (for example, reservoir 20D). In addition, the cylinder (1) is directly connected through the hole (12) to the sample source in the form of an automatic sampling system or sample changer (62) or a pipe (60) through which the stream of the tested (monitored) sample flows, or an airlock (50) on the pipe (60). The use of an airlock (50) is particularly advantageous as it allows to avoids the risk of contamination of the tested sample stream. An airlock (50) is preferably used. The cylinder (1) is also connected via an opening (16) to a waste channel (61) for removing waste fluids from the reaction space. Moreover, the cylinder (1) is also connected through the opening (14) to the detection chamber (6). In the current solution, there is no need to uptake gas from the outside in order to equalise the pressure inside the hydraulic system.

The connection of the cylinder (1) through the holes (10) with the reservoirs (20) in the inverse cartridge (30) is carried out by channels (11) in the housing block (5), ports (28) with stepped undercuts (29) and side-sealing gaskets (27), in the housing block (5), cooperating with the movable lid (26), receiving the through pins (25) at the outlet of the channels (24), leading to sockets (23) of the dispensing tips (22) of the reservoirs (20) in the cartridge (30). The connection of the cylinder (1) to the sample source (50,60,62) is through the opening (12) and the channel (13), while its connection to the waste channel (61) is through the opening (16) and the channel (17). The channels (13,17) are equipped with quick connect fittings (40,43) at the outlet, respectively, accepting hoses (41,44). Preferably, the quick couplings (40,43) are of the FESTO/SMC type, mounted in threaded holes in the housing block (5), which end the channels (13,17) on the outer surface of the housing block (5), preferably on the bottom surface, preferably on the tips sticking out of the housing block (5). The connection of the cylinder (1) to the detection chamber (6) in the detection block (7) is made through the opening (14) and the channel (15). The detection chamber (6) is further connected to the waste channel (61) through the channel (18) in the detection block (7), equipped at the outlet with a quick connect fitting (45), preferably of the FESTO/SMC type, mounted in a threaded cavity in the detection block (7), ending the channel (18) on the outer surface of the detection block (7), preferably the bottom surface, receiving a hose (46), further connecting to the hose (44) or running to the waste channel (61). The hoses (41,44,46) are made of a flexible, rigid, chemically inert material, for example perfluorinated polymers, preferably poly(tetrafluoroethylene) (PTFE), fluorinated ethylene propylene (FEP) or NAFION (a copolymer of tetrafluoroethylene and perfluorinated oligovinyl ether terminated with a sulfone group).

The housing block (5) has external dimensions ensuring the stability of the reaction area, i.e. its rigidity, dimensional stability and tightness of the cylinder (1) with the pistons (2). The dimensions of the housing block (5) ensure free movement of the pistons (2) with the connectors (4). The housing block (5) is preferably a cuboid with a length of 85-105 mm, a width of 25-40 mm and a height of 25-80 mm, preferably the housing block (5) has dimensions of 94×28×27 mm in the variant with a separate detection block (7) (FIG. 39) or 94×28×57 mm in the variant with an integrated detection block (7) (FIG. 40). It is possible to use the housing block (5) in a different shape, but this unnecessarily complicates the production process and the way of mutual arrangement of the functional elements of the device inside its housing. The housing block (5) has a through-opening, preferably with a circular cross-section, forming a cylinder (1) from which through-channels (11,13,15,17) extend towards the reservoirs (20) in the cartridge (30), the sample source (50,60,62), the detection chamber (6) and the waste channel (61), respectively. The channels (11,13,15,17) have a diameter in the range of 0.8-2.0 mm, preferably 1.0 mm, and are preferably perpendicular to the axis of the cylinder (1).

The detection block (7) has external dimensions ensuring the stability of the detection area, i.e. stiffness, dimensional stability and tightness of the detection chamber (6) with windows (8). The detection block (7) is preferably in the form of a cuboid with a length of 30-50 mm, a width of 25-40 mm and a height of 25-40 mm, preferably the cuboid detection block (7) has dimensions of 28×28×30 mm. Alternatively, the detection block (7) is in the form of a cylinder with a diameter of 20-50 mm and a height of 25-40 mm, preferably this cylindrical detection block (7) has a diameter of 28 mm and a height of 30 mm. The cylindrical detection block (7) is particularly advantageous in the variant with two detectors (74,75) and separable blocks (5,7). It is possible to use the detection block (7) in a different shape, but it unnecessarily complicates the production process and the way of mutual arrangement of the functional elements of the device inside its housing. The detection block (7) has at least one through-hole, preferably two perpendicular through-holes, preferably with a circular cross-section, closed with transparent windows (8), constituting a detection chamber (6). A preferred variant of the detection chamber (6) with two perpendicular openings allows for the use of an optical detection system (70) with two detectors (74,75), one of which (74) lies on the axis of the optical path (72) of the light source (71) on the opposite side of the detection block (7), and the other (75) on the axis of the optical path (73) perpendicular to the axis of the optical path (72), which allows for photometric, turbidimetric, fluorine metric or nephelometric, while the single-opening variant allows the use of an optical detection system (70) with one detector (74) located on the axis of the optical path (72) of the light source (71), which allows only photometric or turbidimetric detection. The detection chamber (6) has an internal diameter in the range of 3-10 mm, preferably 4-6 mm, most preferably 4 mm. The windows (8) are made of a chemically inert material, transparent in the range of determination of the product of the specific reaction, preferably they are transparent in the range of visible light, near infrared and near ultraviolet, which ensures the possibility of conducting of a variety of specific reactions. For example, the windows (8) are made of acrylic glass (PMMA), polycarbonate (PC) or polystyrene (PS). The detection block (7) also has through channels (15,18), preferably coaxial, one of which (15) connects the detection chamber (6) with the cylinder (1), and the other connects the detection chamber (6) with the waste channel (61). The detection block (7) is made of a rigid chemically inert material, preferably polyetheretherketone (PEEK), acrylic (PMMA), poly(acrylonitrile-co-butadiene-co-styrene) (ABS) or polyamide (PA). It is also possible to manufacture the detection block (7) from metal, for example stainless steel or aluminium, wherein aluminium is used only if alkaline reagents are not to be used.

The diameter of the holes (10,12,14,16) in the cylinder wall (1) is equal to the diameter of the channels (11,13,15,17,18) coaxial with them in the housing block (5) and the detection block (7). Preferably, the diameter of these holes is 0.8-2 mm, preferably 1.0 mm. Preferably, all holes (10,12,14,16), channels (11,13,15,17,18), through elements of other parts of the device (24,25) and hoses (41,44,46) have the same internal diameter, in the range of 0.8-2.0 mm, preferably 1.0 mm.

Preferably, the detection block (7) is a separate element from the housing block (5). Thanks to the separation of these blocks (5,7), it is possible to simplify the production process, and thanks to material savings also to reduce the production costs. What is more, the separation of the blocks (5,7) allows manufacturing them in various shapes, for example, a rectangular housing block (5) and a cylindrical detection block (7). During the operation, the detection block (7) is located in the seat in the housing block (5) and is rigidly and detachably connected with it, preferably through the screws passing through the dedicated through openings in the detection block (7), embedded with a thread in the threaded holes in the housing block (5). Between the housing block (5) and the detection block (7) there is a flat gasket (19) made of an elastic chemically inert plastic, preferably made of silicone, polyurethane or ethylene-propylene-diene monomers rubber (EPDM). Channel (15), connecting the cylinder (1) in the housing block (5) with the detection chamber (6) in the detection block (7) runs partly in both blocks (5,7). Its initial fragment runs in the housing block (5), and the final fragment runs in the detection block (7), while both fragments of the channel (15) retain coaxial and have the same diameter. The tightness of the connection of both fragments of the channel (15) is provided by using a gasket (19), which has a hole coaxial with the channel (15) and of the same diameter thereof.

Alternatively, the detection block (7) is permanently connected to the housing block (5), preferably made of a single element or two elements glued together. The channel (15) connecting the cylinder (1) in the housing block (5) with the detection chamber (6) in the detection block (7) runs partly over both of these blocks (5,7). Its initial fragment runs in the housing block (5), and the final fragment runs in the detection block (7), while both fragments of the channel (15) remain coaxial and have the same diameter. This solution has the advantage that it ensures tightness of the connection of both blocks (5,7) without the need to using a gasket (19), which reduces the possible failure of the system by ensuring complete tightness of the channel (15) along its entire length. In addition, the permanent integration of the blocks (5,7) simplifies the assembly process.

The holes (10,12,14,16) are located in different parts of the cylinder (1) in order to ensure that the fluid can be transferred to each of the channels (11,13,15,17), coaxial with them, separately. The distance between the projections of the axes of the holes (10,12,14,16) on the axis of the cylinder (1) equals 10-14 mm, preferably 11 mm. The spacing of the holes (10,12,14,16) at a distance of 11 mm is advantageous due to the optimisation of the working volume of the cylinder (1) while ensuring its relatively small beneficial diameter of 6 mm. Using 11 mm spacing of the holes (10,12,14,16) with a preferred internal diameter of 1.0 mm, with hoses supplying the sample (41) and draining the post-reaction mixture (46) and waste solutions (44) with a preferred internal diameter of 1 mm and a preferred external diameter of 4 mm, the maximum volume of the working space between the pistons (2), ensuring contact with a single hole (10,12,14,16) in the cylinder (1) with a diameter of 6 mm equals 621 μl, which corresponds to the opening of the pistons (2) by 22 mm, and the maximum working volume for moving the pistons in hydraulically and pneumatically neutral conditions is 310 μl, which corresponds to the opening of the pistons (2) by 11 mm, i.e. the opening ensuring exposure to a single hole (10,12,14,16) in each position of the pistons (2) spaced apart in this way. By using one of the reservoirs (20D) in the cartridge (30) as a place for storing and mixing the working solution, it is possible to carry out determinations using a working solution with a maximum volume of 10,000 μl, i.e. the nominal volume of the reservoir (20D) in the cartridge (30), however, this volume is unfavourable due to the limited possibility of mixing solutions in the reservoir. The practical maximum volume is preferably around 1000-1200 μl. To ensure adequate working space inside the cylinder (1), its length equals 85-105 mm, preferably 94 mm. The use of the cylinder (1) with a favourable length has the advantage that it allows the use of commercially available lead screws of the stepper motors (3) with a standard length of 200 mm, without the need to modify them, which greatly simplifies the production process and reduces its costs.

The cylinder (1) can be placed in the device at any angle in relation to the surface, but the variant of horizontal orientation is considered to be particularly advantageous.

The holes (10,12,14,16) of the cylinder (1) are coaxial with the channels (11,13,15,17,18) in the housing block (5) and the detection block (7), with the holes and channels in pairs (10-11, 12-13, 14-15, 16-17) having the same diameter in pairs. Both blocks (5,7) are machined, preferably with classical techniques, by boring the cylinder (1), the detection chamber (6) and then the system of the channels (11,13,15,17,18) with properly designed outer outlets, i.e. ports (28) with stepped undercuts (29) at the outlet of the channels (11) and threaded holes receiving quick connect fittings (40,43,45) respectively at the outlets of the channels (13,17,18). It is possible to produce these elements using other techniques, for example additive techniques, but classical machining (cutting, turning, hollowing, etc.) is the cheapest and fastest while maintaining the desired precision and quality of finish. In the variant with blocks (5,7) made of a single piece of material, a ready-made functional element is obtained. In turn, in the variant with detachable blocks (5,7), they are put together using a gasket (19) and, for example, screwed together. And in the variant with glued blocks (5,7), they are put together and permanently joined with the use an adhesive, for example methacrylic, epoxy, polyurethane glue.

The reaction-detection system according to the invention, in variant III, has a replaceable inverse cartridge (30) containing at least four reservoirs (20A,20B,20C,20D) to store chemical reagents and liquids necessary for carrying out the determinations. Construction as well as the technical and analytical functions of the cartridge (30) in the preferred variant Ill are the same as in variant II of the reaction-detection system according to the invention, and the following description applies to both of these variants.

The reaction-detection system, in variant III, has a replaceable inverse cartridge (30) containing at least four reservoirs (20) to store the chemical reagents and to provide space for temporary storage of the working solution and its mixing. The first of the reservoirs (20A), at the stage of preparation to the determination process, is filled with a portion of a standard solution, which is used to carry out calibration measurements. The calibration measurement, preferably at least two-point measurement, is carried out at the beginning of the measurement sequence, using a clean matrix solution devoid of analyte taken from the sample source (50,60,62) and the standard solution (ST) stored in the first reservoir (20A). Two subsequent reservoirs (20B,20C) are filled with chemical reagents (R1,R2) necessary to carry out the specific reaction, which reagents often cannot be stored in one vessel due to their mutual chemical instability. It is also possible to use these reservoirs to store reagents for various specific reactions. The fourth reservoir (20D), according to the invention, is used for temporary storage of the working solution and mixing (MIX) of its components sequentially drawn into the cylinder (1). The use of the reservoir (20D) as a mixer eliminates the problem of accidental drawing the solutions from other reservoirs (20A,20B,20C) during the movement of the pistons (2) in the cylinder (1), and also increases the volume of the working solution, which is no longer limited by the spacing of the holes (10,12,14,16) of the cylinder (1), which possibly allows, without analytical consequences, to reduce the spacing of these holes (10,12,14,16), and also allows for obtaining an accurate mixing the components of the reaction solution. It is possible to use more reservoirs (e.g. 20E,20F,20G, etc.) in the cartridge (30) for further chemicals if required, however, four reservoirs are sufficient for the vast majority of standard applications. Each of the reservoirs (20) is connected to the cylinder (1) by its own dedicated channel created by successive elements (10,11,28,25,24,22,20) lying between the cylinder (1) and the reservoirs (20).

The reservoirs (20) are preferably in the form of commercially available syringes with pistons (21), preferably set with the dispensing tips (22) pointing downwards. Thanks to the use of commercially available syringes, it is possible to choose them for a specific application, for example, syringes certified for medical applications can be safely used in the device in the variant for dialysis monitoring. In addition, the use of commercially available syringes without their modification significantly reduces the production time, which results in the reduction of the cost of production, assembly and further operation of the device. The volume of the reservoirs (20) can be arbitrary, but it must be large enough to provide enough reagent volume for the planned series of determinations (for example, 20-50 determinations), at the same time it should be relatively small to ensure the compactness of the cartridge (30) and the device itself. According to the invention, the reservoirs (20) have a volume of 5-12 ml, preferably 10 ml, with a height in the range of 75-90 mm, preferably 84 mm, which is the size of 10 ml syringes. In order to reduce the dimensions of the cartridge (30), the pistons (21) are used with the retainer removed, which allows the pistons (21) to move completely freely in the reservoir (20) without increasing the overall vertical dimension of the reservoir (20) with the piston (21). The reservoirs (20) have dispensing tips (22), preferably located centrally in their bottom end, preferably LUER tips. The closure in the form of a piston (21) ensures easy pumping of the solution into the reservoirs (20) and its uptake from the reservoirs (20), forced by changes in the pressure inside the hydraulic system, generated by the movement of the pistons (2) in the cylinder (1). The reservoirs (20) are rigidly and tightly seated by the dispensing tips (22), preferably of the LUER type, in compatible sockets (23), preferably of the LUER or LUER LOCK type, at the bottom, inside the housing (31) od the cartridge (30).

The replaceable inverse cartridge (30) is in the form of a container consisting of at least a housing (31), a lid (32) and a lock (33), preferably a one-time latch. Preferably, the cartridge (30) is disposable, and the role of the housing (31,32) and the lock (33) is to protect the reservoirs (20) against accidental damage or removal, and the solutions stored in them against contamination or spillage. The cartridge (30) is preferably in the form of a cuboid with a length of 100-150 mm, a width of 40-50 mm and a height of 90-120 mm, preferably the cartridge (5) has dimensions of 136×41×95 mm, and the housing body (31,32) of the cartridge (30) has a width of 90-110 mm, preferably 100 mm. It is possible to use the cartridge (30) in a different shape, but this unnecessarily complicates the manufacturing process and the way it is connected to the ports (28) on the housing block (5). The construction material of the cartridge (30) is a thermoplastic material, preferably poly(acrylonitrile-co-butadiene-co-styrene) (ABS), polycarbonate (PC), polystyrene (PS) or polyamide (PA).

The housing (31) of the cartridge (30) has a system of elements that allows the connection of the reservoirs (20) and lossless pumping of solutions between the cylinder (1) and these reservoirs (20). The housing (31) of the cartridge (30) has internal sockets (23), preferably of the LUER or LUER LOCK type, receiving the dispensing tips (22), preferably of the LUER type, of the reservoirs (20), preferably four reservoirs (20A,20B,20C,20D). In turn, the sockets (23) are connected by channels (24), favourably with a diameter equal to the diameter of the holes (10) and channels (11), equipped at their outlet on the lower outer surface of the housing (31) with flight stem (25), compatible with ports (28) with stepped undercuts (29) and side-sealing gaskets (27), preferably of shape of an O-RING torus, at the outlet of the channels (11) at the outer surface of the housing block (5) of the cylinder (1). The through pins (25) have an outer diameter in the range of 2.5-8 mm, preferably 6 mm, and a height in the range of 5-20 mm, preferably 16 mm. The ports (28) have an internal diameter of 2.7-8.2 mm, preferably 6.2 mm, and a depth of 6.2-21.2 mm, preferably 17.2 mm, while the depth of the stepped undercut equals 4-6 mm, preferably 5 mm, and its diameter is adapted to the diameter of the gasket and equals 6.25-14.25 mm, preferably 10.25 mm. The torus-shaped O-RING gasket (27) has a diameter D/d in the range of 4/1-8/3 mm, preferably 6/2 mm.

Between the cartridge (30) and the housing block (5), at the outlet of the channels (11) in the outer surface of the housing block (5) of the cylinder (1), there is a movable pressing lid (26), which role is to press the gasket (27) while docking the pins (25) of the cartridge (30) in the ports (28) of the housing block (5). The lid (26) preferably has through holes accepting the through pins (25), while from the side of the housing block (5) it has protrusions around these holes, which, when docking the cartridge (30), enter the ports (28) in the area of the stepped undercut (29) and press the gasket (27) sealing the connection of the through pins (25) with the ports (28), while the other surface of the lid (26) rests against the bottom surface of the housing (31) of the cartridge (30). To ensure the compression of the gasket (27), the lid (26) has a height greater by 0.5-2.0 mm, preferably 0.75 mm, than the difference between the length of the pins (25) and the thickness of the gasket (26), in a preferred variant it equals 12.7 mm. According to the invention, the inner diameter of the through holes of the lid (26) is 0.2 mm larger than the outer diameter of the pins (25) and equals 2.7-8.2 mm, preferably 6.2 mm.

The housing (31) of the cartridge (30) additionally has the side sockets (34) receiving the forks (35) of the lift (36) of the device, ensuring automatic movement and docking of the cartridge (30) in the ports (28). In addition, the housing of the cartridge (30) has vertical through holes (37), preferably two holes in the bottom of the housing (31), for receiving positioning pillars (38) during docking, which pillars (38) stuck upright at the housing block (5). The positioning pillars (38) are preferably located between the ports (28B,28C) at the housing block (5), and are made of metal and have a tapered end, which facilitates their penetration into the holes (37). The role of the positioning pillars (38) is precise, repeatable and correct positioning of the cartridge (30) mounted on the forks (35) of the lift (36) while docking the pins (25) in the ports (28). The positioning pillars (38) have an outer diameter in the range of 4-6 mm, preferably 4.85 mm, and a height in the range of 10-40 mm, preferably 25 mm. The lift (36) moves on two vertical linear guides and is set in motion by a dedicated stepper motor equipped with a lead screw. The forks (35) are made of one bent metal element, fixed in four points on the lift (36), which allows for maintaining the rigidity of the forks (35). The part of the fork (35), remaining in contact with the housing (31,32) of the cartridge (30), has the shape of a horizontal cuboid ending with a trapezoidal tip widening towards the end, extending beyond the front surface of the cartridge (30) when correctly docked, with the front wall of the housing (31,32) of the cartridge (30) resting on these tips. The height of the trapezoidal ends of the forks (35) is smaller than the height of the socket (34) by 4-10 mm, preferably 7 mm, and the inner width of the forks (35) is larger than the width of the housing body (31,32) of the cartridge (30) by 0.2-1.0 mm, preferably 0.5 mm, which ensures easy placing of the cartridge (30) on the forks (35) of the lift (36) while stabilising its position in the vertical axis, even before sliding the positioning pillars (38) into the holes (37) in the cartridge (30). Preferably, the forks (35) are made of a metal plate with a thickness of 2-5 mm, preferably 3 mm, a width of 8-15 mm, preferably 10 mm, and their internal spacing is 90.5-110.5 mm, preferably 100.5 mm, and the height of the trapezoidal fork tips equals 15-25 mm, preferably 20 mm, with the sockets (34) 3-7 mm wide, preferably 4.5 mm, and 22-32 mm high, preferably 27 mm. The process of docking the cartridge (30) in the housing block (5) considers lowering the lift (36) to the lower position. Then, the forks (35), which in the upper position support the cartridge (30) resting on them with their sockets (34), press the cartridge (30) downwards, ensuring the seating of the pins (25) in the ports (28).

The cartridge (30) has an electronic system (39) equipped with a non-volatile memory (NFC RFID chip) ensuring its wireless connectivity. The electronic system (39) wirelessly communicates with the antenna of the electronic main controller (88) of the device when the cartridge (30) is docked in the device. The non-volatile memory of the cartridge (30), having a unique serial number, coding the analyte and the type of the specific reaction supported by this cartridge, is recognised by the electronic main controller (88) of the device, which, according to the applied algorithm of operation, allows the cartridge (30) to be used only once. This is possible in various ways, for example by erasing the non-volatile memory of the electronic system (39) of the cartridge (30) or by saving the number of the used cartridge (30) in the database to which the device connects during operation.

The optical detection system (70) has the same mode of operation and similar characteristics as in variants I and II described above. The differences are subtle and result from differences in the design of the detection chamber (6), as well as from the use of a CCD matrix with RGB filters. The elements of the optical detection system (70) are located around the detection chamber (6) oriented towards the interior thereof. The optical path (72), connecting the light source (71) and the detector (74), passes with its entire width through the interior of the detection chamber (6), preferably through the crossing point of the axis of the opening/openings constituting the detection chamber (6) with the axis of the channels (15,18). In turn, the optical path (73), running to the detector (75), perpendicular to the optical path (73), passes with its entire width through the interior of the detection chamber (6), preferably through the crossing point of the axis of the opening/openings constituting the detection chamber (6) with the axis of the channels (15,18), where the optical axes of the optical paths (72,73) also preferably intersect.

The optical detection system (70) consists of a light source (71), for example in the form of a diode, a fluorescent lamp or a light bulb, oriented with the front towards the interior of the detection chamber (6), and two detectors (74,75), for example in the form of a diode, a photodiode, a photoresistor, a photomultiplier tube, a CCD array or a CMOS array. The first detector (74), for photometric or turbidimetric detection, oriented towards the interior of the detection chamber (6), is located on the axis of the optical path (72) of the light source (71) on the opposite side of the detection chamber (6). The second detector (75), for fluorimetric or nephelometric detection, oriented towards the inside of the detection chamber (6), is located on the axis of the optical path (73) intersecting at an angle of 90° with the axis of the optical path (74) of the light source (71). The light source (71) and detectors (74,75) are preferably mounted directly around the detection chamber (6), but can also be brought to the desired location via optical fibres. The width of the optical paths (72,73) is 1-10 mm, preferably 4 mm, and the axes of the optical paths (72,73) pass through the interior of the detection chamber (6), preferably with their entire width, and preferably cross at the geometrical intersection of the axis of the opening/openings constituting the detection chamber (6) with the axis of the channels (15,18). The present invention provides four forms of construction of the optical detection system (70) for variant Ill described herein.

The optical detection system (70) has a light source (71) emitting light of an adjustable wavelength, preferably equipped with a monochromator, while the detector (74) is a CCD matrix, and the detector (75) is a CCD matrix or a universal fluorimetric LED detector (FIG. 45). Alternatively, the optical detection system (70) has a light source (71) emitting white light with a continuous spectrum, while the detector (74) is a CMOS matrix and the detector (75) is a CMOS matrix or a universal fluorimetric LED detector (FIG. 46). Both of these forms of embodiments are equipped with a universal light source and universal detectors, thanks to which they can be used to determine the product of any specific reaction. However, these systems are expensive and complicated, which may exclude the possibility of their routine use for repeated determinations of one analyte.

Preferably, the optical detection system (70) has a light source (71) emitting monochromatic light in the range of absorption or excitation of the product of the specific reaction, preferably a LED diode, while the detector (74) is a CCD matrix, preferably with an RGB filter, or a specific LED detector with characteristics adapted to the specific analyte, and the detector (75) is a CCD matrix, preferably with an RGB filter, or a universal fluorimetric LED detector (FIG. 47). Alternatively, the optical detection system (70) has a light source (71) emitting monochromatic light of several wavelengths in the range of absorption or excitation of the product of the specific reaction, preferably an integrated SMD diode equipped with several LEDs, where the optical axes (72) of these diodes pass through the point of geometric intersection of the axis of the opening/openings forming the detection chamber (6) with the axis of the channels (15,18), while the detector (74) is a CCD matrix, preferably with an RGB filter, or several specific LED detectors with characteristics adapted to specific analytes, and the detector (75) is a CCD matrix, preferably with an RGB filter, or a universal fluorimetric LED detector (FIG. 48). Both of these embodiments have a light source (71) adapted to the determination of a specific analyte and universal detectors, thanks to which they can be used only to determine the product of a specific reaction. This functionality is suitable for routine use with repeated determinations of a single analyte.

Most preferably, the optical detection system (70) has a light source (71) emitting monochromatic light of several wavelengths in the range of absorption or excitation of the product of the specific reaction, preferably the light source (71) is an integrated SMD LED diode equipped with several LED diodes, while the optical axes (72) of these diodes pass through the point of geometric intersection of the axis of the opening/openings forming the detection chamber (6) with the axis of the channels (15,18), while the detectors (74,75) are universal CCD matrices, preferably with RGB filters (FIG. 28). This solution ensures the functional flexibility necessary for routine determinations of selected analytes and very good quality of the obtained data with the simplicity of construction and low production costs.

The above-described embodiment of the present invention can be characterised as follows:

1. A reaction-detection system with a replaceable cartridge for a device for automated determination of an analyte in the liquid phase by conducting a specific chemical reaction and subsequent optical measurement of the concentration of its products, in particular for monitoring the progress of the dialysis process, constituting an element of a hydraulic system equipped with a network of channels for pneumatic pumping of liquid solutions, wherein a single cylinder acts as a reaction space and an optical detection space, which cylinder has walls transparent in the range of determination of the product of the specific reaction, which cylinder is equipped with two opposing coaxial movable pistons closing it on each side, moved by electronically controlled stepper motors equipped with lead screws with positioning nuts, which are connected to the pistons by dedicated connectors, making the pistons to move linearly inside the cylinder, which cylinder is directly connected by separate channels/hoses to at least four reagent reservoirs in a replaceable cartridge, a sample source and a waste channel, and the determination of the analyte is carried out in an optical detection system for the determination of the product of the specific reaction, consisting of from a detection chamber equipped with transparent walls for the determination of specific reaction products, a light source and a detector/detectors for photometric, turbidimetric, fluorimetric or nephelometric detection, which is located on the axis of the optical path of the light source facing the interior of the detection chamber, where the light source and the detector are placed around the detection chamber, and the optical path connecting the light source and the detector passes through its interior, in which arrangement, according to the invention, the cylinder (1), with two pistons (2) moved by stepper motors (3) connected to them via connectors (4), constitutes a reaction space in the housing block (5), which cylinder (1) has holes (10,12,14,16) connected to the channels (11,13,15,17) in the housing block (5), respectively, which holes and channels in pairs (10-11, 12-13, 14-15, 16-17) have the same diameter, and each of at least four holes (10) and respective channels (11), are detachably connected to one reservoir (20) in the cartridge (30) through ports (28) with a stepped undercut (29), equipped with a side-sealing gaskets (27) and a compression lid (26), detachably receiving through pins (25) of the cartridge (30), connected by channels (24) with the sockets (23) detachably receiving the dispensing tips (22) of the reservoirs (20), while the hole (12) and the channel (13) equipped with the quick connect fitting (40) is detachably connected by the hose (41) with the sample source, i.e. with the automatic sampling system (62), the pipe (60) with the sample stream or the airlock (50) on the pipe (60), while the hole (16) and the channel (17) equipped with the quick connect fitting (43) is detachably connected by the hose (44) with the waste channel (61), while the hole (14) by the channel (15) is connected to the detection chamber (6) in the form of a transverse opening in the detection block (7), sealed from the outside with transparent windows (8) cooperating with the elements of the optical detection system (70), while the detection chamber (6) through the channel (18) equipped with a quick connect fitting (45) is detachably connected with the hose (46) to the hose (44) or the waste channel (61).

2. In the present, preferred embodiment of the reaction-detection system with a replaceable cartridge, the cylinder (1) is a through-opening in the housing block (5), preferably with a circular cross-section, with an internal diameter in the range of 3-8 mm, preferably 4-7 mm, most preferably 6 mm, and a length in the range of 85-105 mm, preferably 94 mm, and its pistons (2) with a compatible external diameter in the range of 3.2-8.2 mm, preferably 4.2-7.2 mm, most preferably 6.1 mm, tightly embedded inside the cylinder (1), have piston rods made of inert chemically rigid plastic material, such as poly(ethylene terephthalate) (PTFE), polyether ether ketone (PEEK), poly(acrylonitrile-co-butadiene-co-styrene) (ABS), polyamide (PA), or polypropylene (PP), optionally in configuration with a gasket, preferably flat, made of mono ethylene-propylene-diene rubber (EPDM), silicone or polyurethane, and the rod guides are made of metal such as brass, aluminium or steel.

3. Preferably, the cylinder (1) is oriented horizontally, while the diameters of the holes (10A,10B,10C,10D) lie at the intersection of the vertical plane passing through the axis of the cylinder (1) with the side surface of the cylinder (1) located above its axis, and the diameters of the holes (12,14,16) are located at the intersection of this plane with the side surface of the cylinder (1) below its axis, wherein the upper holes (10) and the lower holes (12,14,16) are located alternately, with their preferred sequence in any direction: 10A, 12, 10B, 14, 10C, 16, 10D, and the distance between the projections of their axes on the axis of the cylinder (1) equals 10-14 mm, preferably 11 mm, and the diameter of the holes (10,12,14,16) is 0.8-2.0 mm, preferably 1 mm.

4. According to the invention, the housing block (5) has the form of a cuboid with a length of 85-105 mm, a width of 25-40 mm and a height of 25-80 mm, preferably the housing block (5) has dimensions of 94×28×27 mm in the variant with a separate detection block (7) or 94×28×57 mm in the variant with an integrated detection block (7), wherein the housing block (5) has through channels (11) connecting the holes (10) of the cylinder (1) with the ports (28), while the through channels (13,17) connect the holes (12,16) of the cylinder (1) with quick connect fittings (40,43), while the through channel (15) connects the hole (14) of the cylinder (1) with the detection chamber (6), where the channels (11,13,15,17) have a diameter in the range of 0.8-2.0 mm, preferably 1 mm, and are preferably perpendicular to the axis of the cylinder (1), while and the housing block (5) is made of a rigid chemically inert material, preferably polyetheretherketone (PEEK), acrylic (PMMA), poly(acrylonitrile-co-butadiene-co-styrene) (ABS), polyamide (PA), aluminium or stainless steel.

5. The detection block (7) has the form of a cuboid with a length of 30-50 mm, a width of 25-40 mm and a height of 25-40 mm, preferably a rectangular detection block (7) has dimensions of 28×28×30 mm, or the detection block (7) has the form of a cylinder with a diameter of 20-50 mm and a height of 25-40 mm, preferably a cylindrical detection block (7) has a diameter of 28 mm and a height of 30 mm, while the detection block (7) has at least one through-hole forming a detection chamber (6), preferably with a circular cross-section, with an internal diameter in the range of 3-10 mm, preferably 4-6 mm, most preferably 4 mm, closed with transparent windows (8) made of a chemically inert material which is transparent in the range of determination of the product of the specific reaction, preferably acrylic glass (PMMA), polycarbonate (PC) or polystyrene (PS), as well as a through channel (15) connecting the cylinder (1) with the detection chamber (6), and a through channel (18) connecting the detection chamber (6) with the quick connect fitting (45), wherein the channels (15,18) having a diameter in the range of 0.8-2 mm, preferably 1 mm, and preferably perpendicular to the axis of the detection chamber (6), and the detection block (7) is made of a rigid chemically inert material, preferably polyetheretherketone (PEEK), acrylic (PMMA), polyamide (PA), poly(acrylonitrile butadiene styrene) (ABS), aluminium or stainless steel.

In particular, in this arrangement, the detection chamber (6) is formed of two perpendicular, through openings, preferably perpendicular to the channels (15,18), preferably with a circular cross-section, with an internal diameter in the range of 3-10 mm, preferably 4-6 mm, most preferably 4 mm, closed with windows (8).

The detection block (7) is rigidly and detachably connected to the housing block (5), preferably with screws, while the channel (15) connecting the cylinder (1) with the detection chamber (6) runs partly in the housing block (5) and partly in the detection block (7) maintaining coaxiality of both its sections, and between the housing block (5) and the detection block (7) there is a gasket (19) with a hole coaxial with the channel (15), of the same diameter, made of elastic chemically inert material, preferably silicone, polyurethane or ethylene-propylene-diene monomer rubber (EPDM).

Alternatively, the detection block (7) is permanently connected to the housing block (5), preferably it is made of one element or two elements glued together, and the channel (15) connecting the cylinder (1) with the detection chamber (6) runs partly in the housing block (5) and partly in the detection block (7) maintaining the coaxiality of both its sections.

6. In the current reaction-detection system with a replaceable cartridge, the hoses (41,44,46) are made of a chemically inert material, preferably poly(tetrafluoroethylene) (PTFE), fluorinated ethylene propylene (FEP) or NAFION (a copolymer of tetrafluoroethylene and perfluorinated oligovinyl ether terminated with a sulfone moiety), and have an internal diameter in the range of 0.8-2.0 mm, preferably 1.0 mm, and the thickness of their walls is 0.5-2.0 mm, preferably 1.5 mm, and are connected to the outlets of the channels (13,17,18) with quick connect fittings (40,43,45), preferably of FESTO/SMC type, equipped with a flat gasket made of an elastic chemically inert material, preferably ethylene-propylene-diene monomers rubber (EPDM), silicone or polyurethane (PU), mounted in threaded holes at the outlets of the channels (13,17,18).

7. The source of the sample in the present solution is a classic sampling system (62) in the form of an automatic sample changer, where the tip (42) of the hose (41) or its extension, at the time of sampling, is placed in a vessel filled with the test sample, or the sample source is a pipe (60) with the sample stream, in which the rigid tip (42) of the hose (41) is placed with the opening directed upstream of the sample stream, or the sample source is an airlock (50) through which the sample stream passes from the pipe (60) with the sample stream, preferably the sample is taken from the accumulation reservoir (52) of the airlock (50) or its waste channel, and the end (42) of the hose (41) is directed with the opening downwards or upwards, respectively.

In the particular case, when monitoring the progress of the dialysis process, the source of the sample is the airlock (50) on the pipe (60) with the dialysate stream flowing directly from the haemodialysis machine, and the end (42) of the hose (41) is located in the accumulation reservoir (52).

8. In this preferred embodiment, the cartridge (30) has at least four reservoirs (20A,20B,20C,20D), preferably in the form of syringes with pistons (21), made of chemically inert materials, preferably polypropylene (PP), preferably with a valid medical device certificate, with a volume in the range of 5-12 ml, preferably 10 ml, with dispensing tips (22), preferably of the LUER type, preferably located in the centre at the axis of the reservoirs (20), embedded in a detachable manner with the outlet downwards in the sockets (23), preferably of the LUER or LUER LOCK type, at the bottom inside the housing (31) of the cartridge (30).

9. In addition, the cartridge (30) has the form of a container consisting of at least a housing (31), a lid (32) and a lock (33), preferably disposable, wherein the housing (31) of the cartridge (30) has a socket (23) at the inner bottom, preferably four sockets (23A,23B,23C,23D), preferably of the LUER or LUER LOCK type, each receiving one dispensing tip (22), preferably of the LUER type, of the reservoirs (20A,20B,20C,20D), which sockets (23) are connected by channels (24), preferably with a diameter equal to the diameter of the holes (10) and channels (11), equipped at the outlet on the bottom outer surface of the housing (31) of the cartridge (30) with through pins (25) with the outer diameter of 2.5-8 mm, preferably 6 mm, and a height of 5-20 mm, preferably 16 mm, compatible with ports (28) with an internal diameter of 2.7-8.2 mm, preferably 6.2 mm, and depth 6.2-21.2 mm, preferably 17.2 mm, where the depth of the stepped undercut (29) equals 4-6 mm, preferably 5 mm, and its diameter equals 6.25-14.25 mm, preferably 10.25 mm, having inside the side-sealing gaskets (27), preferably in the shape of an O-RING torus, with a diameter D/d of 4/1-8/3 mm, preferably 6/2 mm, and the lid (26) at the outlet of the channels (11) in the outer top surface of the housing block (5) of the cylinder (1), wherein the lid (26) is 0.5-2.0 mm higher, preferably 0.75 mm higher, than the difference in the height of the pin (25) and the thickness of the gasket (27), and one plane of the lid (26) rests against the bottom surface of the housing (31) of the cartridge (30), while the opposite plane of the lid (26) presses a gasket (27) when docking the cartridge (30), sealing the connection of the pin (25) with the port (28), while the housing (31,32) of the cartridge (30) additionally has side sockets (34) for the forks (35) of the lift (36), made of one bent metal element four-point fixed on the lift (36), which forks (35) have the shape of a horizontal cuboid ended with a trapezoidal tip stabilising the front of the cartridge (30), and also, the cartridge (30) has vertical through holes (37) for the posts (38) positioning the cartridge (30) in relation to the housing block (5), ensuring automatic movement, repeatable vertical movement of the cartridge (30) mounted on the forks (35) of the lift (36), as well as the correctness and repeatability of docking of the through pins (25) of the cartridge (30) in the ports (28) of the housing block (5) of the cylinder (1), and the construction material of the cartridge (30) is a thermoplastic, preferably polycarbonate (PC), polystyrene (PS), polyamide (PA) or poly(acrylonitrile-butadiene-co-styrene) (ABS).

10. In addition, the cartridge (30) has an electronic system (39) equipped with a non-volatile memory (NFC RFID TAG), wirelessly connected to the antenna of the electronic main controller (88) of the device when the cartridge (30) docked in the device, which memory is recognised by the main controller (88), allowing the given cartridge (30) to be used once.

11. In this preferred embodiment, the optical detection system (70) consists of a light source (71), for example in the form of a diode, a fluorescent lamp or a light bulb, oriented towards the interior of the detection chamber (6), and optionally one detector (74) or two detectors (74,75), e.g in the form of a diode, photodiode, photoresistor, photomultiplier, CCD matrix or CMOS matrix, one of which (74), for photometric or turbidimetric detection, oriented towards the interior of the detection chamber (6), is located on the axis of the optical path (72) of the light source (71) on the opposite side of the detection chamber (6), and the other (75), for fluorimetric or nephelometric detection, oriented towards the interior of the detection chamber (6), is located on the axis of the optical path (73) intersecting at an angle of 90° with the axis of the optical path (73) of the light source (71), wherein the light source (71) and detectors (74,75) can be brought to the desired place via optical fibres, while the width of the optical paths (72,73) equals 1-10 mm, preferably 4 mm, and the axes of the optical paths (72,73) pass through the interior of the cylinder (1), preferably with their entire width, and preferably crossing at the point of geometric intersection of the axis of the opening/openings forming the detection chamber (6) with the axis of the channels (15,18).

Furthermore, the optical detection system (70) has a light source (71) emitting light of an adjustable wavelength, preferably equipped with a monochromator, while the detector (74) is a CCD matrix and the detector (75) is a CCD matrix or a universal fluorimetric LED detector, or the optical detection system (70) has a light source (71) emitting white light with a continuous spectrum, while the detector (74) is a CMOS matrix and the detector (75) is a CMOS matrix or a universal fluorine a metric LED detector, or the optical detection system (70) has a light source (71) emitting monochromatic light in the range of absorption or excitation of the products of the specific reaction, preferably an LED, while the detector (74) is a CCD matrix, preferably with an RGB filter, or a specific LED detector with characteristics adapted to a specific analyte, and the detector (75) is a CCD matrix, preferably with an RGB filter, or a universal fluorimetric LED detector, or the optical detection system (70) has a light source (71) emitting monochromatic lights of several wavelengths in the range of absorption or excitation of the product of the specific reaction, preferably an integrated SMD LED equipped with several LEDs, wherein the optical axes (72) of these LEDs are parallel and lie on the axis of the SMD LED cover, coinciding with the axis of the opening forming the detection chamber (6), while the detector (74) is a CCD matrix, preferably with an RGB filter, or several specific LED detectors with characteristics and the detector (75) is a CCD matrix, preferably with an RGB filter, or a universal fluorimetric LED detector.

In the special case, the optical detection system (70) for monitoring the progress of the dialysis process, adapted to the determination of creatinine, urea and phosphate ions, has an integrated light source (71) in the form of a SMD LED emitting monochromatic radiation in the range of 500-550 nm, preferably 525 nm, for the determination of adduct of creatinine with picric acid, in the range of 410-460 nm, preferably 415 nm, for the determination of add urea with 4-(dimethylamino)benzaldehyde and in the range of 550-900 nm, preferably 625 nm, for the determination of phosphomolybdenate blue, and has universal detectors (74,75) in the form of CCD matrices with RGB filters and channels working in the red, green and blue light ranges.

12. This example of implementation of the present invention in the field of the reaction-detection system with a replaceable cartridge is used in the method of automated determination of an analyte in the liquid phase by conducting a specific chemical reaction and subsequent optical measurement of the concentration of its products, using the device for automated determination of the analyte in the liquid phase by conducting a specific chemical reaction and subsequent optical measurement of the concentration of its products, especially for monitoring the progress of the dialysis process, in which a specific chemical reaction tailored to a specific analyte and the wavelength for the determination of the product of the specific reaction is selected, then the device is adapted to the selected determination by adjusting the optical detection system, and the content of the cartridge is adjusted by filling the first reservoir with the analyte standard solution and the next two reservoirs with chemical reagents necessary to carry out the specific reaction, and then the cartridge is placed in the device, after which a portion of the solution to be determined is sampled by dragging its portion through the hose to the cylinder, and then portions of chemical reagents are sequentially taken from the two reservoirs of the cartridge, into the cylinder, the reaction solution is mixed, and then the reaction solution is transferred to the area of the optical detection system, ensuring the level of liquid that allows the optical path of the light source to pass through the solution to be determined, preferably the level of the solution completely covering the optical path, after which, after a certain time, the concentration of the specific reaction product is optically determined using an optical detection system by photometric, turbidimetric, fluorimetric or nephelometric measurement, or their combination, after which the post-reaction solution is pumped out to the waste channel, and then the cylinder is cleaned by washing it with a fresh portion of the tested solution drawn into the cylinder through the sampling hose, which is then pumped out of the cylinder into the waste channel, with the fluid flow in the hydraulic system being pneumatically generated by changing the relative mutual position of the pistons in in the cylinder, and in cases where it is necessary to move the reaction solution to the desired area of the cylinder, the pistons are moved in a hydraulically and pneumatically neutral mode with the same speed, direction and return inside the cylinder, while monitoring the dialysis progress, when the analyte concentration readings indicate a deviation from the expectations for a given analyte, or when the assumed analytical effect is achieved, the alarm system is activated, in which method, according to the invention, the device comprises a reaction and detection system with a replaceable cartridge described above, where the fourth reservoir (20D) in the cartridge (30) serves as a mixer, and all solutions are sequentially drawn into the cylinder (1) during the determination of the tested sample, i.e. sample from the source samples (60,62,50), matrix solution or standard solution from the reservoir (20A) and reagents from the reservoirs (20B,20C) are pumped to the mixer (20D) immediately after being drawn into the cylinder (1), and after all solutions have been drawn and pumped to the fourth reservoir (20D), the resulting reaction solution is mixed by pumping it between the cylinder (1) and the reservoir (20D), while the volume of the tested sample equals 30-90 μl, the volume of the reagents used equals 50-250 μl, giving the reaction mixture with a volume of 240-320 μl, and after mixing the reaction solution, its portion, preferably 240 μl, is pumped from the mixer (20D) to the cylinder (1), and then it is pumped through the hole (14) and channel (15) to the detection chamber (6), where the analyte is determined, and after the determination, the reaction solution is pumped out of the detection chamber (6) and the reservoir (20D) through the channel (18) and the channel (17), respectively, to the waste channel (61).

### Airlock

According to the invention, the device may be equipped with an airlock (50) protecting the source of the analytical material against its contamination by the device. The airlock (50) is particularly useful when samples for testing are taken from a liquid stream (60) where there is an upstream system which is sensitive to microbial contamination. There is a risk of contaminants being carried upstream along the walls of vessels and hoses used in the system, especially if the device is not disinfected between the tests. This is particularly important when the source of the sample requires antiseptic treatment, which is of a particular importance in medical applications in regard to patient well-being, for example, when monitoring the composition of dialysate flowing through the waste channel of a haemodialysis machine during the ongoing process of blood dialysis of the patients suffering from renal failure. Microbiological safety is ensured by physical breaking the walls' continuity of the channel (60) through which the tested sample flows, so that there is no risk of contaminants migrating along the walls of this channel upstream to the sample stream, which would result in contamination of the sample source. The airlock (50) enables the safe use of the device for the automated determination of analytes in the liquid phase, according to the invention, for the analysis of the dialysate of various patients without the need to disinfect the device after each patient.

According to the invention, the airlock (50) is a module in which the walls of the channel (60) carrying the flowing sample stream are physically discontinuous on a specific section of the stream, i.e. on a given fragment, the stream of the tested liquid flows without physical contact with the walls of the pipe and it is surrounded by air. The airlock (50) is placed on the channel (60) with the stream of the tested sample, above the sampling point with hose (41).

The airlock (50), according to the invention, in the basic embodiment (FIG. 55), has the form of a closed module, receiving from the top the ending fragment of the channel (60), carrying the stream of the flowing tested liquid. The liquid flows down into the main reservoir (51) located below, capable of holding a certain amount of the sample (e.g. 50 ml). The reservoir (51) has two electronically controlled valves at the bottom, one (55) for draining the fluid into the waste channel (61), and the other (54) for draining the fluid into the channel with a sampling system (41). The airlock (50) is optionally equipped with an overflow channel (53) to maintain the flow of the stream in the event of power failure (FIG. 56A). The overflow channel (53) is separated from the main reservoir (52) by a barrier which height determines the effective volume of the main reservoir (51). During operation, the airlock (50) periodically accumulates the fluid in order to uptake a portion of the sample by the sampling hose (41), and is emptied after the sampling.

In another embodiment (FIG. 56B), the main reservoir (51) of the airlock (50) is divided into two half-chambers separated by a partition with a height lower than the barrier separating the main reservoir (51) from the overflow channel (53). A separate half-chamber forms an accumulation reservoir (52). Then, the main reservoir (51), equipped with a valve (55), is located under the outlet of the channel (60), thanks to which it is filled in the first place, while the accumulation tank (52), equipped with a valve (54), is located on the side, thanks to which it is filled only after the liquid has been poured over the barrier separating the half-chambers (51,52). The separation of the main reservoir (51) has the advantage that the accumulator reservoir (52) can hold a portion of the solution for some time after the main reservoir (51) has been emptied, allowing a given determination to be repeated if necessary.

In the preferred embodiment (FIG. 57), the airlock (50) is a module placed on the sample stream channel (60) to ensure the microbiological safety of the sample source located upstream of the channel (60). This is particularly important when the source of the sample requires an antiseptic treatment, which is of particular importance in medical applications in regard to patients' well-being, for example, when monitoring the composition of dialysate flowing through the waste channel of a haemodialysis machine during the ongoing process of blood dialysis of patients suffering from renal failure. Microbiological safety is ensured by physical breaking the walls' continuity of the channel (60) through which the tested sample flows, so that there is no risk of the contaminants to migrate along the walls of this channel upstream to the sample stream, which would result in contamination of the sample source. The airlock (50) enables the safe use of the device for the automatic determination of the analyte in the liquid phase, according to the invention, for the analysis of the dialysate of various patients without the need to disinfect the device after each patient.

The sample stream channel (60) has an outlet into the main reservoir (51) of the airlock (50), i.e. the sample stream flows without physical contact with any part of a pipe, hose or other structural element of the airlock (50) or the channel (60). Preferably, the walls of the sample stream channel (60) are not in contact neither with the top lid nor with wall of the main reservoir (51). The liquid flows into the located below main reservoir (51), capable of holding a sample volume of 1000-4400 ml, preferably 1000 ml. The main reservoir (51) has an outlet connected to the waste channel (61). At the waste channel there is a valve (55) electronically controlled by the main controller (88) of the device for the automatic determination of the analyte in the liquid phase, according to the invention, wherein the outlet of the main reservoir (51) to the waste channel (61) passes through a sealed hole in the bottom lid of the main reservoir (51). Collecting the portion of the sample from the sample stream channel (60), while averaging its composition over a certain period of time, allows to determine its temporary averaged composition. The main reservoir (51) is periodically filled and periodically emptied, which ensures a continuous flow of the sample stream from the sample stream channel (60) to the waste channel (61).

The main reservoir (51) is equipped with an overflow channel (53) connected to the waste channel (61). This ensures smooth flow of the sample stream from the channel (60) through the main reservoir (51) to the waste channel (61) even in the event of the valve (55) failure, the main controller (88) failure or the power failure. The point of the overflow of the liquid to the overflow channel (53) is located below the outlet of the sample stream channel (60) to the main reservoir (51), thanks to which it is not possible to fill the main reservoir (51) in such a way that the fluid level inside this reservoir would exceed the level of the outlet of the sample channel (60), which would involve the risk of contamination of the sample source located upstream of the channel (60). In turn, the relative height of the overflow point from the main reservoir (51) to the overflow channel (53) determines the effective capacity of the main reservoir (51). Preferably, when using a construction pipe with an internal diameter of 100-160 mm, preferably 110 mm, the location of the overflow point at a relative height of 50-120 mm from the bottom of the main reservoir (51), preferably 105 mm, without taking into account other installations inside the main reservoir (51), ensures its capacity of approximately 475-1140 ml, preferably 1000 ml.

Preferably, the overflow channel (53) is located inside the main reservoir (51). This aims at simplification the construction and reducing the amount of external elements that are susceptible to damage. In addition, the placement of the overflow channel (53) inside the housing of the main reservoir (51) intends to increase the reliability of the airlock (50) and reduce the risk of leakage to the environment. The overflow channel (53) is in the form of a pipe with a diameter in the range of 10-75 mm, preferably 32 mm. The further outlet of the overflow channel (53) to the waste channel (61) passes through a sealed hole in the bottom lid of the main reservoir (51). The overflow point from the main reservoir (51) to the overflow channel (53) is in the form of an opening in the side wall of the structural pipe of the overflow channel (53) or takes the form of an end of this pipe open at the top.

According to the invention, the airlock (50) is equipped with an accumulation reservoir (52) filled in an overflow manner from the main reservoir (51). The overflow point from the main reservoir (51) to the accumulation reservoir (52) is located below the overflow point to the overflow channel (53), thanks to which the priority of filling the accumulation reservoir (52) is ensured, and its content being not disturbed during an emergency overflow through the overflow channel (53). At the bottom, the accumulation reservoir (52) has an outlet to the waste channel (61) equipped with a valve (54), electronically controlled by the main controller (88) of the device, according to the invention. The relative height of the overflow point from the main reservoir (51) to the accumulation reservoir (52) determines the effective capacity of this reservoir (52). Preferably, when using a construction pipe with a diameter of 10-75 mm, preferably 32 mm, the location of the overflow point at a relative height of 40-105 mm, preferably 95 mm, from the bottom of the accumulation reservoir (52), ensures its effective capacity of about 26-67 ml, preferably 60 ml.

The role of the accumulation reservoir (52) is to store a certain portion of the tested solution with a constant composition in order to enable reliable determination of the analyte. According to the invention, the accumulation reservoir (52) is overflow-filled from the main reservoir (51) after it has been filled to the level allowing the overflow. According to the invention, during operation, the accumulation reservoir (52) with the valve (54) closed is periodically filled to the level resulting from its construction, preferably it is filled to its 100% volume, which in the preferred embodiment results in accumulation of 60 ml of the tested solution. After the planned fill-up of the accumulation reservoir (52), the main reservoir (51) is emptied by opening the valve (55) and draining all the solution accumulated there into the waste channel (61). Then, the main reservoir (51) can be refilled with the tested solution according to the planned measurement regime. In the meantime, the determination of the analyte in a portion of the solution collected in the accumulation reservoir (52) is carried out, ensuring the stability of its composition.

Preferably, the accumulation reservoir (52) is located inside the main reservoir (51). This aims at simplification of the construction and reducing the number of external elements that are susceptible to damage. In addition, the placement of the accumulation reservoir (52) inside the housing of the main reservoir (51) intends to increase the reliability of the airlock (50) and to reduce the risk of leakage to the environment. The accumulation reservoir (52) is in the form of a pipe with a diameter in the range of 10-75 mm, preferably 32 mm. The outlet from the accumulation reservoir (52) to the waste channel (61), through the valve (54), passes through a sealed hole in the bottom lid of the main reservoir (51). The overflow point from the main reservoir (51) to the accumulation reservoir (52) is in the form of an opening in the side wall of the construction pipe of the accumulation reservoir (52) or takes the form of an open top end of this construction pipe.

According to the invention, sampling the tested solution from the accumulation reservoir (52) takes place by drawing its portion by the hose (41) placed inside thereof, preferably in the axis of the accumulation reservoir (52) outlet. This positioning of the hose (41), passing through the opening in the top lid of the main reservoir (51) or in the side wall of the main reservoir (51) above the overflow point into the overflow channel (53), ensures that there is no risk of leakage when using the airlock (50) and when dumping the stored liquids, because the hose (41) passes through the housing of the main reservoir (51) in the area of the airlock (50) that is not filled with fluid and therefore does not require sealing. According to the invention, sampling is carried out in a stationary mode, and the content of the accumulation reservoir (52) is reduced with each sampling only by the volume of the solution drawn through the hose (41) to the device according to the invention. This allows for an effective reduction of the volume of the accumulation reservoir (52) while ensuring the possibility of multiple repetition of determinations of a given portion of the solution, as required by analytical needs, such as the observed clear deviation between the recorded result and the expected value, failure of the detection system or other circumstances suggesting the need of repetition of the measurement. According to the invention, the preferred volume of the accumulation reservoir (52) is 60 ml, which means that when it is 100% full, it is possible to freely carry out five determinations with the device according to the invention, which allows routine determination of samples with a preferred volume of 10 ml, taking into account the technical steps of rinsing the reaction-detection system before and after the analysis.

The preferred design of the accumulation reservoir (52) and the stationary mode of the sampling method constitutes an improvement over the other embodiments of the airlock (50) described above, where the sampling was done in flow mode by the hose (41) located in the outlet of the accumulation reservoir (52) below the valve (54), while the solution from the accumulation reservoir (52) was drained into the waste channel (61). This was associated with low sampling efficiency and high loss of the tested sample during the sampling process. This resulted in the need to provide of a relatively large volume of the accumulation reservoir (52), and allowed for no more than one repetition of the determination of a given portion of the solution in the event of an analytical necessity.

In the present preferred embodiment, discharge of the waste solutions from the device for automatic analyte determination in the liquid phase, according to the invention, takes place by the hose (44) placed in the overflow channel (53), preferably in the axis of its outlet. This positioning of the hose (44), passing through the opening in the top lid of the main reservoir (51) or in the side wall of the main reservoir (51) above the overflow point from the main reservoir (51) to the overflow channel (53), ensures no risk of leakage when using the airlock (50) and when dumping the stored liquids, because the hose (44) passes through the housing of the main reservoir (51) in the area of the airlock (50) that is not filled with fluid and therefore does not require sealing. In addition, the possibility of accidental drawing the solution from the overflow channel (53) into the device for the automatic determination of analytes in the liquid phase, according to the invention, is practically excluded, because the overflow channel (53) does not have the capacity to accumulate the solution, and the nature of its use (natural overflow from the main reservoir in emergency situations with unrestricted drainage) precludes its filling under the operation conditions of the airlock (50). This is a significant advantage over other embodiments, where the discharge takes place through a hose (44) placed in the waste channel of the main reservoir (51) or accumulation reservoir (52), which required appropriate sealing of the passage point of the hose (44) through a fragment of the housing of the waste channel of the airlock (50), and was also associated with the risk of accidental uptake of the waste solution drained from the main reservoir (51) or accumulation reservoir (52) to the device for automatic determination of the analyte in the liquid phase, according to the invention.

Outflow from the main reservoir (51), accumulation reservoir (52) and overflow channel (53) to the waste channel (61) takes place through their waste channels. Preferably, the waste channels of the main reservoir (51) and the accumulator reservoir (52) merge into one channel below their valves (54,55) and then join the waste channel of the overflow channel (53) to form a waste channel (61).

The airlock (50), its rigid and flexible structural elements, are made of materials that are chemically inert and durable in the environment of the tested sample, preferably durable in aqueous and corrosive environment, for example, rigid elements are made of plastics such as polyvinyl chloride (PVC), poly(acrylonitrile-co-butadiene-co-styrene) (ABS) or polypropylene (PP), while the flexible elements are made of plastics such as poly(tetrafluoroethylene) (PTFE), fluorinated ethylene propylene (FEP) or a terpolymer obtained from ethylene propylene diene monomers (EPDM). Similarly, the valves (54,55) are made of chemically inert materials, for example PP or EPDM. Preferably, the entire airlock (50), all its structural elements and the elements connecting it with the device for the automatic determination of analytes in the liquid phase, according to the invention, are made of rigid elements in order to ensure maximum reliability of the system and to keep the geometry and internal volume constant over time. However, this does not preclude, for construction reasons, the possibility of justified use of flexible elements, especially such as PP or EPDM hoses.

According to the invention, the airlock (50) is equipped with a pressure sensor (56), preferably a differential pressure sensor, for sensing the flow rate of the dialysate through the airlock (50). This is an important parameter which monitoring allows for the detection of abnormalities in the functioning of the sample stream source, preferably in the functioning of the haemodialysis machine, which is extremely important for ensuring the well-being of patients undergoing blood dialysis treatment. The pressure sensor (56) is equipped with a rigid pneumatic hose (57) with an internal diameter of 1-10 mm, preferably 4 mm, which open end is located vertically downwards inside the main reservoir (51), preferably at its bottom. Due to this positioning, the pressure readings change as the main reservoir (51) fills-up, with the filling rate of the main reservoir (51) being proportional to the flow rate of the dialysate from the haemodialysis machine. The hose (57) passes through an opening in the top lid of the main reservoir (51) or in the side wall of the main reservoir (51) above the overflow point to the overflow channel (53), i.e. in the area of the airlock (50) that is not filled with fluid and therefore does not require sealing, which ensures that fluid cannot leak from the airlock (50) even in an emergency situation. The pressure sensor (56) is connected to the electronic main controller (88) of the device for the automatic determination of the analyte in the liquid phase, according to the invention, which ensures the transfer of information about the dialysate flow to the operator of this device, who simultaneously monitors the operation of the haemodialysis machine.

The airlock (50) is also equipped with a temperature sensor (58), preferably a PT 100 resistance temperature sensor or a thermocouple, placed in the main reservoir (51) at its bottom. This allows for the control of the dialysate leaving the haemodialysis machine and allows for possible detection of abnormalities in its functioning, which is extremely important for ensuring the well-being of the patients undergoing blood dialysis treatment. The temperature sensor (58) is connected to the electronic main controller (88) of the device for the automated determination of the analyte in the liquid phase, according to the invention, which ensures the transmission of information about the dialysate flow to the operator of this device, who simultaneously monitors the operation of the haemodialysis machine. The output of the temperature sensor signal (58) goes through a sealed hole in the wall of the main reservoir (51) or its bottom lid, or through an unsealed hole in the top lid of the main reservoir (51) or in the side wall of the main reservoir (51) above the point of its overflow to the overflow channel (53).

According to the invention, the airlock (50) is an open system, since sealing of the system when the drain valves (54,55) are closed would push the solution through the overflow channel (53) and pressurise the airlock (50) system, which may cause the haemodialysis machine to initiate an alarm due to the inability to drain the dialysate. The possible production of a sealed construction of the airlock (50) would require its deliberate unsealing above the overflow point from the main reservoir (51) to the overflow channel (53), thanks to which the system would become open and the whole structure would not be exposed to leakage even in emergency situations.

The airlock (50), according to the invention, has been designed to cooperate with the device for the automatic determination of the analyte in the liquid phase, according to the invention, especially for monitoring changes in the composition of the dialysate during the process of blood dialysis of the patients with renal failure. Preferably, the sample stream channel (60) is the haemodialysis machine waste channel. However, it is possible to use it in other measuring systems or in combination with other systems that require microbiological safety while ensuring a continuous outflow of the sample stream from the system sensitive to contamination.

The solution, according to the present invention, regarding the airlock (50) can be characterised as follows:

1. An airlock (50) for the device for the automatic determination of the analyte in the liquid phase by conducting a specific chemical reaction and the subsequent optical determination of the concentration of its products, especially for monitoring the progress of the process of blood dialysis, is a module on the sample stream channel, for example on the outflow channel of a haemodialysis machine, containing an outlet of the sample stream channel inside the main reservoir, ensuring a physical break of the continuity of the walls of the sample stream channel and its temporary flow in the surrounding of air, which main reservoir is equipped with an overflow channel connected to the waste channel, with an outlet located below the outlet of the sample stream channel, and is also equipped with an accumulation reservoir filled by overflow from the main reservoir, with the overflow to the accumulation reservoir located below the overflow channel, and equipped with waste channels at the bottom of the main reservoir and at the bottom of the accumulation reservoir, equipped with electronically controlled valves for draining the liquids into the waste channel, with the sampling system of the device for automated determination of the analyte in the liquid phase located below the outlet of the sample stream channel to the main reservoir, in the accumulation reservoir below the level of its overflow, wherein in the module, according to the invention, the end of the hose (41) used for sampling for the purposes of the device for the automatic determination of the analyte in the liquid phase, is located inside the accumulation reservoir (52) at its bottom, above the valve (54) but below the point of overflow fill from the main reservoir (51), where the structural elements of the airlock (50) are made of chemically inert materials and durable in the environment of the tested sample, preferably durable in the aqueous and corrosive environment, for example, rigid elements are made of plastics such as polyvinyl chloride (PVC), polypropylene (PP) or poly(acrylonitrile-co-butadiene-co-styrene) (ABS), while flexible elements are made of plastics such as poly(tetrafluoroethylene) (PTFE), fluorinated ethylene propylene (FEP) or ethylene propylene diene monomer (EPDM).

2. In the airlock (50), the main reservoir (51) has the form of a pipe with an internal diameter in the range of 110-160 mm, preferably 110 mm, with a bottom lid and a top lid, where the sample stream channel (60) and the hose (41) pass through the holes in the top lid of the main reservoir (51), and the accumulation reservoir (52) and the overflow channel (53) are located inside the housing of the main reservoir (51) and preferably have the form of pipes with a diameter in the range of 32-75 mm, preferably 32 mm, with their outlets passing through sealed openings in the bottom lid of the main reservoir (51), while the overflow points from the main reservoir (51) to the accumulation reservoir (52) and the overflow channel (53) are in form of openings in the side walls of the pipes constituting the accumulation reservoir (52) and the overflow channel (53) or their top open ends.

3. In the airlock (50), the end of the hose (44) used to discharge waste liquids from the device for automatic analyte determination in the liquid phase is located inside the overflow channel (53) of the main reservoir (51), in its lower part, below the point of overflow from the main reservoir (51), preferably in the axis of its outlet to the waste channel (61), with the hose (44) passing through the hole in the top lid of the main reservoir (51).

4. The airlock (50) is equipped with a pressure sensor (56), preferably a differential pressure sensor, with a rigid pneumatic hose (57) with a diameter of 1-10 mm, preferably 6 mm, the open end of which is placed in the main reservoir (51), while the hose (57) passes through a hole in the top lid of the main reservoir (51), wherein the pressure sensor (56) is connected to the electronic main controller (88) of the device for the automatic determination of the analyte in the liquid phase.

5. The airlock (50) is also equipped with a temperature sensor (58), preferably a PT 100 resistance temperature sensor or a thermocouple, located in the main reservoir (51) at its bottom, and its output passes through a sealed hole in the wall of the main reservoir (51) or its bottom lid, which temperature sensor (58) is connected to the electronic main controller (88) of the device for the automatic determination of the analyte in the liquid phase.

6. Furthermore, the airlock (50) has a waste channel (61) connected to the waste channels of the main reservoir (51), the storage reservoir (52) and the overflow channel (53) of the main reservoir (51), preferably the waste channels of the main reservoir (51) and the accumulation reservoir (52) merge into one channel above the connection to the waste channel of the overflow channel (53).

7. The airlock (50) is an open system and the walls of the sample stream channel (60) are preferably not in contact with the housing of the main reservoir (51).

8. In a special case, in the current airlock (5), the sample stream channel (60) is the waste channel of a haemodialysis machine, which eliminates the risk of microbial contamination of the haemodialysis machine and increases the microbiological safety of the patients undergoing blood dialysis treatment.

### Alarm System

The device is also equipped with an alarm system (80) consisting of a speaker (81), a light source (82) and means of remote communication (83). The alarm system (80) is controlled by the main controller (88). The system (80) automatically activates an appropriate message in situations requiring the operator's attention, e.g. after achieving the expected analytical effect or in the event of deviation of the analytical result from the expectations in relation to a given determination. The situations listed here are extremely important due to the correctness of the determinations. In such situations, an appropriate sound and light signal, preferably set by the operator, is activated on the device (e.g. a beep and a green light indicating the end of the measurement or a whistle and a red light indicating an error), and an appropriate information is sent to peripheral devices, preferably to the operator's telephone.

### Electronic Main Controller

The electronic main controller (88) equipped with antennas for remote communication is a programmable logic controller which is used to control the entire device according to the invention. The main controller (88) has been adapted to control the device by introducing an appropriate algorithm into its memory that allows for the sequential execution of subsequent steps of the process of automated analyte determination, including, i.a. detection of a specific analyte by recognising the RFID chip of the cartridge (30), control of the operation of the stepper motor of the lift (36), control of the stepper motors (3) driving the pistons (2) in the cylinder (1) in a way that allows for calibration and a series of determinations, as well as control the operation of the optical detection system (70), alarm system (80), external communication with the terminal device (e.g. external computer), and optionally the operation of the airlock (50) or the sample changer (62).

### Method of Automated Determination of an Analyte in the Liquid Phase

According to the invention, a method of automated determination of an analyte in the liquid phase by conducting a specific chemical reaction and subsequent optical measurement of the concentration of its products uses the device described above, in a suitably selected variant. According to the invention, the determination of a specific analyte in a sample with specific characteristics requires preliminary development and optimisation of the entire determination method. What is extremely important, the method according to the invention is used in situations where it is impossible to easily determine the analyte by other methods, classically used in flow analysis, and when the analyte is not a coloured substance that can be determined optically. For example, it is possible to monitor the process of blood dialysis by monitoring the changes in the concentration of various uremic toxins in the dialysate stream (creatinine, urea, phosphate ions). In order to avoid confusion of the end user, the cartridges (30/90) may have different colours or large easily recognisable graphical markings to distinguish different types of cartridges for different analytes and their respective specific reactions.

In the first step, an appropriate specific reaction (or a series of reactions) is being selected, that converts the specific analyte into a coloured form, and then the optimal wavelength for the determination of the product of this specific reaction is selected, taking into account matrix effects that may interfere the determination. Then, the device, according to the invention, is adapted to a specific determination by selecting an appropriate light source (71), as well as selecting an appropriate number and a content of the reservoirs (20/95) in the cartridge (30/90). When preparing the device, in the variant with four reservoirs (20/95), the first reservoirs (20A/95A) is filled with an analyte standard solution (ST), and the next two reservoirs (20B/95B,20C/95C) with reagents necessary to perform the specific reaction. The fourth reservoir (20D/95D) performs various functions depending on the version of the device. In variant I with the normal cartridge (90), the reservoir (95D) is filled with a matrix solution (MATRIX), or it is left empty until the solution is drawn from the sample source (50,60,62), while in variants II and Ill with the inverse cartridge (30), the reservoir (20D) functions as a mixer (MIX). Optionally, the ready-made disposable cartridges (30/90) are used, factory-prepared for the specific determinations. The cartridge (30/90) is then placed in the device. If it is necessary to sample the matrix solution after placing the cartridge (30/90) in the device, a portion of it is drawn from the sample source (50,60,62) through the hose (41) to the cylinder (1). Then, the taken matrix solution is used for calibration measurement (variants II and III) or this portion of the matrix solution is pumped into the reservoir (95D) through the hole (10D) and the hose (99D), and the process is repeated until the desired level of the solution in the reservoir (95D) is reached (variant I). The calibration is supplemented with the measurement of the standard solution (ST) from the reservoir (20A/95A). During the determination, a portion of the solution to be determined is sampled from the sample source (50,60,62) by drawing its portion through the hose (41) to the cylinder (1), and then the appropriate portions of chemical reagents (R1,R2) stored in the reservoir (20B/95B,20C/95C) are sequentially collected. Upon completion of a specific determination, liquid waste is discharged through the hose (44) to the waste channel (61). According to the invention, sequential measurements are carried out according to the needs and analytical assumptions.

When planning the determination, concentrations of reagents and standards should be appropriately selected, and an appropriate matrix solution should be provided. Reagent concentrations are selected so that the volumes of reagents used are as small as possible, so that the reagent would not run out at high concentrations and volumes of the analyte, while at the same time large enough to enable efficient dosing of the reagent at low concentrations of the analyte. Dispensed volumes of the reagents can be adjusted to the current concentration of the analyte during subsequent determinations within one series, assuming monotonic changes in the concentration of the analyte in the sequence or stream. Concentrations are preferably selected in a way that takes into account the stoichiometry of the conducted reactions, so as to draw the same portions of the reagents, which excludes faster depletion of one of them.

According to the invention, sampling may be conducted using a standard sample changer (62), using a channel with the sample stream (60), or using and airlock (50) installed on the channel (60) through which the test sample stream flows. Using the airlock (50) protects the source of the analytical material against contamination by the device. The airlock (50) is of particular use when the tested samples are taken form the stream having upstream the source which is sensitive to microbiological contamination. There is a danger of migration of the contaminations upstream along the walls of vessels and hoses of the system, especially when the device is not disinfected between the determinations. This is particularly important during the monitoring of post-dialysis liquid flowing out of the haemodialysis machines, due to the need of maintaining these machines sterile to ensure well-being of the patients undergoing blood dialysis treatment. The airlock (50) allows the device to be used to analyse the dialysate of various patients without a need of disinfecting the having after each patient. Preferably, the portion of the test sample is taken from the accumulation reservoir (52) or its wate channel, which temporarily holds a portion of the test sample, which allows its repeated determination when a failure of the device is observed or the analytical results deviate from the expectations.

According to the invention, the alarm system (80) is equipped with a speaker (81), a light source (82) and means of remote communication (83) automatically sending a message about the achievement of the desired analytical effect or about deviations of the analytical result from the expectations in relation to a given measurement, automatically triggering the appropriate sound and light signal on the device, and sending the information about the achievement of the assumed analytical effect to peripheral devices such as a display on the device or the operator's phone, preferably equipped with a dedicated mobile application. Thanks to this, it is easier to control the course of determinations, especially when conducting various parallel processes. The alarm system allows to avoid the negative effects associated with it with the occurrence of anomalies during the determination as well as optimisation of the device's operating time.

### The Method of Determination of the Analyte Using the Device in Variant I

The fluid flow in the hydraulic system is generated pneumatically by changing the relative position of the pistons (2) in the cylinder (1) when the hole (14) is outside the working space between the pistons (2). In cases where it is necessary to supplement or to reduce the amount of gas in the working space between the pistons (2), without generating liquid flow in the hydraulic system, the pistons (2) are moved when the hole (14) is located in the space between the pistons (2). On the other hand, in cases where it is necessary to move the reaction solution to the desired area of the cylinder (1), the entire working space between the pistons (2) is transferred in a hydraulically and pneumatically neutral mode, moving the pistons (2) with the same speed in the same direction to the appropriate area of the cylinder (1).

During the determination, a portion of the solution to be determined is sampled from the sample source (50,60,62) by dragging its portion through the hose (41) to the cylinder (1), and then the appropriate portions of chemical reagents are sequentially taken from the reservoirs (95B,95C) in the cartridge (90) to the cylinder (1). When necessary, gas is refilled or reduced in the working space between the pistons (2) using the hole (14) and the hose (47) or the holes (10) and the hoses (99). The volume of the reaction solution is preferably 428-1040 μl in the system with a horizontal cylinder (1) and a distance between the projections of the holes on its axis (10A,12,10B,14,10C,16,10D) of 5 mm. Alternatively, the volume of the reaction solution is 565-1040 μl using a vertical cylinder (1) system with a 5 mm distance between the projections of the holes on its axis (12,14,10A,14,10B,14,10C,14,10D,14,16). Preferably, the reaction solution is mixed by passing gas through the reaction solution and drawing it into the cylinder (1) from the hole (14). After the reaction is initiated, the reaction solution is moved between the pistons (2) to the area of the appropriate optical detection system (70), with properties adapted to the characteristics of the determined substance. Before the determination, the height of the portion of the solution in the cylinder (1) between the pistons (2) is raised to the level ensuring the passage of the optical path (72,73) through the tested solution, preferably to the level completely covers the optical path (72,73). Then, after a certain time, the concentration of the product of the specific reaction product is determined optically using the optical detection system (70) by photometric, turbidimetric, fluorimetric or nephelometric measurement, or a combination thereof, depending on the needs and the expected achievable analytical effect. Upon completion of the optical determination, the reaction solution between the pistons (2) is moved in the neutral mode to the outlet of the hose (44) and it is pumped out of the cylinder (1) into the waste channel (61). Next, the cylinder (1) is cleaned by washing it with matrix solution from the reservoir (95D) or with a portion of fresh test solution of the current composition drawn from the sample source (50,60,62), which is then pumped from the cylinder (1) to the waste channel (61).

According to the invention, the measurement for the actual sample is preceded by calibration measurements using the standard solution from the reservoir (95A) and the matrix solution from the reservoir (95D). Preferably, the calibration measurements are repeated during the sequence of the determinations.

Conducting the specific reactions and subsequent optical determination of their products requires precise pumping of fluids in the hydraulic system, which in turn requires precise control of the position of the pistons (2) in the cylinder (1) and precise control of fluid levels in the reservoirs (95).

According to the invention, the position of the pistons (2) is controlled by an electronic main controller (88), controlling the entire device, by moving the stepper motors (3) equipped with lead screws and positioning nuts connected to the connectors (4). The number of steps of the stepper motors (3) and the thread pitch of the lead screws directly determine the precision and resolution of the movement of the pistons (2), which in turn translates into the precision of controlling the volume of the working space of the cylinder (1) between the pistons (2), available for the reaction solution. It is possible to use any stepper motors (3), lead screws and positioning nuts, preferably the same for both pistons (2). Due to the ease of calculation and simulation of the position of the pistons (2), stepper motors (3) with the number of steps of 200/360°, equipped with lead screws with a thread pitch of 1 mm with compatible positioning nuts, are preferably used. Such parameters of the stepper motors (3), applied to the hydraulic system with a cylinder (1) with a preferred diameter of 12 mm, translate into the ability to manage the fluids with an accuracy of 565 nl, which is about 0.1% of the minimum volume of the reaction solution (428 μl in a system with a horizontal cylinder, 565 μl in a system with a vertical cylinder) and ensures sufficient precision of control of the system. The use of a stepper motor (3) with larger number of steps and a lead screw with smaller pitch allows to increase the resolution of the control of the working volume of the cylinder system (1).

The process of drawing the fluids from the reservoirs (95) and the sample source (50,60,62), according to the invention, is generated by creating vacuum in the cylinder (1) in the area of the outlet of one of the hoses (99) and the hose (41). In the variant with a horizontal cylinder (1), the fluids flowing from the hoses (99) drip down the walls of the cylinder (1) and mix with the solution filling the cylinder (1), while the fluid flowing from the hose (41) from the bottom fills the solution in the cylinder (1). Alternatively, in the variant with a vertical cylinder (1), fluids flowing from the hoses (99) and the hose (41) drip down the walls of the cylinder (1) and combine with the solution filling the cylinder (1). In order to avoid uncontrolled outflow of the fluids from the hoses (99) and contamination of the reagents in the reservoirs (95), after the sequence of movements separating the pistons (2) when drawing the fluids from the reservoirs (95) is completed, the pistons (2) slightly slide back, pumping the gas into the hoses (99) until the reagents are completely pushed out of the volume of the hoses (99) back into the reservoirs (95), and then, in hydraulic and pneumatic neutral mode, one of the pistons (2) slides over the outlet of the hose (99), blocking the access to it. Similar injection of gas and subsequent blocking of access to the hose is also carried out in relation to the hose (41) only in the variant with a vertical cylinder (1). In the variant with a horizontal cylinder (1), it is not possible to pump the gas to the hose (41), because its outlet is below the level of the solution in the working space of the cylinder (1). Then, the outlet of the hose (41) is only blocked by the piston (2).

To maintain repeatability of drawing the fluids from the reservoirs (95), gas is pumped into the hoses (99) prior to drawing the fluids until all fluid is pushed out of the hoses (99) into the reservoirs (95). To be sure, the volume of gas greater than the total volume of the hoses (99) is pushed out. Then, it is possible to draw fluids from the reservoirs (95) under repeated conditions of empty hoses (99). When fluid is drawn into the cylinder (1), gas filling the hose (99) is drawn first, followed by the fluid from the reservoir (95). Accurate control of the volume of the fluid being drawn is possible by ensuring that the hose (99), of known volume, is empty prior to drawing. Thus, the pistons (2) move apart to increase the working volume of the cylinder (1) between the pistons (2) by a volume equal to the volume of the hose (99) and the desired volume of fluid from the reservoir (95).

Alternatively, the repeatability of the process of drawing the fluids from the reservoirs (95) is ensured by knowing the current degree of filling of the reservoirs (95) determined by the impedance measurements of the tips (98), which allows the calculation of the degree of filling of the hoses (99) with fluid, the level of which automatically equalises with the level of the fluid in the reservoir (95). When fluid is drawn into the cylinder (1), the gas filling the hose (99) is drawn in first, and only then the fluid drawn from the reservoir (95). Thanks to the precise knowledge of the gas volume in the hose (99), above the current level of the reagent in the reservoirs (95), the pistons (2) move apart in a way that increases the working volume of the cylinder (1) between the pistons (2) by a volume equal to the volume of gas in the hose (99) and the desired volume of fluid to be taken the reservoir (95). Due to the small internal diameter of the hoses (99) of 0.4-2.0 mm, preferably 0.8 mm, the flow of fluids through these hoses (99) occurs through their entire cross-section, so the effects related to the shape of the meniscus inside the hoses (99) are negligible.

After drawing the reagents necessary to carry out the specific reaction to the cylinder (1), further steps should occur after a certain, always the same time before the determination to ensure the same progress of the reaction each time and to maintain repeatability of the determination. At this time, the pistons (2) move the reaction solution to the area of the cylinder (1), where the determination will be carried out using the appropriate optical detection system (70). While the pistons (2) are moving, it is also possible to mix the reaction solution with a stream of gas drawn from the hose (47). During the movement, the pistons (2) position themselves at a distance that elevates the reaction solution to the appropriate level, preferably completely covering the optical path (72,73). The reaction time is determined by the operator based on specific analytical needs and the characteristics of the specific reaction being conducted. When monitoring the changes of the level of the analyte in the solution stream, it is important to ensure the appropriate sampling frequency. For processes lasting 2-5 hours, it is preferable to sample every 5-15 minutes. The reaction time should not exceed half of the sampling period to ensure that the cylinder (1) can be washed before the following determination.

During the reaction, appropriate solutions are mixed together: the tested sample (or matrix solution, or standard solution) and at least one reagent, two reagents, or more reagents, depending on the determination being carried out. The total volume of the reaction solution is selected so as to achieve the appropriate height of the portion of the solution between the pistons (2), ensuring full coverage of the optical path (72,73) along its entire width, which is 1-10 mm, preferably 5 mm. Then, the analytical signal is maximised and the measurement process becomes fully repeatable. As a result, the minimum volume of the reaction solution for determination is smaller when using a horizontal cylinder (1) (FIG. 6A, 6B) than when using a vertical cylinder (1) (FIG. 6C, 6D), because it does not require filling the entire segment of the cylinder (1) of the optical path width (72,73), but only its fragment. For example, using the optical path (72,73) with a favourable width of 5 mm (corresponding to the diameter of a standard diode), the cylinder (1) with a diameter of 12 mm and a distance of 5 mm between the projections of the holes on its axis, the minimum volume of the reaction solution in the variant with the horizontal cylinder (1) equals 428 μl, while in the system with the vertical cylinder (1) it is 565 μl, i.e. 33% more. In turn, the maximum volume of the reaction solution under these conditions is the same in both variants and equals 1040 μl, which directly results from the 5 mm spacing of the holes (10,12,14,16), which means that in order to keep only a single hose accessible to the working space between the pistons (2), their spacing cannot be greater than 9.2 mm. Advantageously, the volume of the reaction solution is minimised, which facilitates handling of the solution and speeds up the measurement.

The small volume of the reaction solution, according to the invention, resulting from the geometry of the cylinder (1), the arrangement of the holes (10,12,14,16) and the optical path width (72,73) is a significant innovation in comparison to the devices known from the state of the art and allows for routine long series of measurements using small volumes of reagents and samples.

When planning the determination, concentrations of reagents and standards should be appropriately selected, and an appropriate matrix solution should be provided. Reagent concentrations are selected so that the volumes of reagents used are as small as possible, so that the reagent would not run out at high concentrations of the analyte, and at the same time large enough to enable efficient dosing of reagents at low concentrations of the analyte. Dispensed volumes of reagents can be adjusted to the current concentration of the analyte during subsequent determinations within one series, assuming monotonic changes in the concentration of the analyte in the sequence or stream. Concentrations are preferably selected in a way that takes into account the stoichiometry of the conducted reactions, so as to draw the same portions of reagents, which excludes faster depletion of one of them.

### The Method of Determination of the Analyte Using the Device in Variant II

The flow of the fluids in the hydraulic system is generated by the mutual movement of the pistons (2) inside the cylinder (1), generating overpressure or underpressure of liquid in a specific part of the hydraulic system, which forces the movement of the liquid to balance the pressure in the system. It should be noted that the sample source (50,60,62) and the waste channel (61), connected to the cylinder (1), are open to the outside environment or are under a protective atmosphere. Unlike the solution in variant I, described above, the reservoirs (20) are sealed. The area of influence of the generated pressure changes in the hydraulic system, understood as the whole device (including channels, cartridge reservoirs, connectors and hoses) connected to the cylinder (1), is limited by the position of the pistons (2) that can move freely inside the cylinder (1). The fluid flow in the system is thus controlled mainly hydraulically. Changing the relative position of the pistons (2) in the cylinder (1) generates the aforementioned pressure changes in the hydraulic system, however, unlike variant I, according to the invention, variant II does not allow refilling or reducing the amount of gas inside the system. In case there is one of the holes (10,12,14,16) in the working space between the pistons (2), the change in the position of the pistons (2) generates the movement of the liquid in the direction that balances the pressure changes, i.e. when the pistons (2) are moved apart, the liquid is sucked into the cylinder (1), and when the pistons (2) are pushed back, the liquid is pushed out of the cylinder (1). It is worth noting that when the pistons (2) move in the cylinder (1) in the same direction with the same speed, the so-called travel in a hydraulically and pneumatically neutral mode, which does not generate the pumping of fluids through the holes (10,12,14,16) and allows the reaction solution to be moved to the desired area of the cylinder (1), for example, to draw a specific reagent, pump the solution to the mixer (20D), pump the solution to the area of the detection chamber (6) or remove the solution from the cylinder (1) to the waste channel (61).

Conducting the specific reaction and subsequent optical determination of its product requires precise pumping of fluids in the hydraulic system, which in turn requires precise control of the position of the pistons (2) in the cylinder (1) and precise control of fluid levels in reservoirs (20).

According to the invention, the position of the pistons (2) is controlled by the electronic main controller (88), controlling the entire device, via the stepper motors (3) equipped with lead screws and positioning nuts connected to the connectors (4) of the pistons (2). The number of steps of the stepper motors (3) and the thread pitch of the lead screws directly determine the precision and resolution of the movement of the pistons (2), which in turn translates into the precision of the control over the volume of the working space of the cylinder (1) between the pistons (2), available for the reaction solution. It is possible to use any stepper motors (3) with any lead screws and positioning nuts, preferably the same for both pistons (2). Due to the ease of calculation and simulation of the position of the pistons (2), stepper motors (3) with the number of steps of 200/3600, equipped with lead screws with a thread pitch of 1 mm with compatible positioning nuts, are preferably used. Such parameters of the set of stepper motors (3), applied to a preferred hydraulic system with a cylinder (1) with a diameter of 6 mm, results in the ability to manage the fluids with an accuracy of 141 nl, which is about 2% of the minimum volume of the tested solution necessary for one-time dosing, which is 7 μl, and ensures sufficiently precise control of the system. The use of a stepper motor (3) with a larger number of steps and a lead screw with a smaller pitch allows for increasing the resolution of the control of the working volume of the cylinder system (1).

The process of drawing the fluids from the reservoirs (20) and the sample source (50,60,62) according to the invention is generated by creating a vacuum in the cylinder (1) in the area of the hole (10) and the hole (12), respectively. The fluids flowing out of the holes (10,12) fill the space between the pistons (2) of the cylinder (1) and possibly combine with the solution filling the cylinder (1). In order to avoid uncontrolled outflow of liquids from the holes (10) and the contamination of the reagents in the reservoirs (20), the reservoirs (20) containing the reagents have a movable piston (21), the movement of which allows the volume of the reservoirs (20) to be reduced under the influence of the negative pressure applied by the pistons (2) moving in the cylinder (1). The neutral movement of the pistons (2) does not cause pressure changes in the reservoirs (20). Contamination of the reagents is also avoided by using a removable membrane on the pins (25) of the cartridge (30), e.g. in the form of a peelable foil which is removed before the cartridge (30) is docked to the device.

In order to maintain the repeatability of the process of drawing the liquids from the reservoirs (20), before starting the measurement cycle, during the preparatory stage, after the cartridge (30) docking, liquids are sequentially drawn from each of the reservoirs (20A,20B,20C), and then the cylinder (1) is rinsed several times with a fresh portion of the currently tested sample. This process allows to standardise the filling of the channels (10,11,28,25,24,22) in the system. Precise control of the volume of the fluid drawn is possible thanks to the certainty that the channels (10,11,28,25,24,22) are always filled to the same extent.

After the reagents, necessary to carry out the specific reaction, are taken to the cylinder (1) and pumped into the mixer (20D), the reaction solution is preferably mixed by pumping it several times between the mixer (20D) and the cylinder (1). During serial determinations, a specific, always the same, amount of time must be allowed before the determination to ensure uniform intervals between the determinations. When determining this time, the time needed to pump the solution from the sample source (50,60,62) and the time needed to clean the cylinder (1), the reservoir (20D) and the detection chamber (6) should also be taken into account.

Before the determination, a portion of the sample with a total volume of 6-12 ml, preferably 8 ml, is taken several times (at least 3 times, preferably 4 times) to wash the cylinder (1) to prepare it for the reaction, and additionally wash the detection chamber (6).

It is important that the detection chamber (6) is filled with the reaction solution during the determination so that the optical path (72,73) is completely covered with the solution to be determined. The reaction time is determined by the operator based on specific analytical needs and the characteristics of the specific reaction being conducted. When monitoring changes in the level of the analyte in the solution stream, it is important to ensure the appropriate sampling frequency. For processes lasting 2-5 hours, it is preferable to sample every 5-15 minutes. The reaction time should not exceed half of the sampling period to ensure that the cylinder (1) can be washed before the following determination.

During the determination, appropriate solutions are mixed together: the tested sample (or matrix solution, or standard solution during the reference measurements) and at least one reagent, two reagents, or more reagents, depending on the test. The total volume of the reaction solution is set to achieve the appropriate volume of the reaction solution to fill the detection chamber (6) and ensure full coverage of the optical path (72) over its entire width, which is 1-10 mm, preferably 4 mm. Only then, the analytical signal is maximised and the measurement process becomes repeatable. Using a detection chamber (6) with a volume in the range of 85-285 μl, preferably 113 μl, a reaction solution with a volume of at least twice as much, preferably 240 μl, is prepared to ensure that the cylinder (1) in the area of the detection chamber (6) is rinsed and completely filled with the tested solution in this region. Preferably, depending on the assay, the volume of the tested sample is 30-90 μl, the volume of reagents used is 50-250 μl, and the resulting reaction mixture has a volume of 240-320 μl. Preferably, the volume of the reaction solution is minimised, which facilitates the manipulation of solutions, speeds up the measurement and reduces the volume of collected reagents.

### The Method of Determination of the Analyte Using the Device in Variant III

The flow of the fluids in the hydraulic system is generated by the mutual movement of the pistons (2) inside the cylinder (1), generating overpressure or underpressure of liquid in a specific part of the hydraulic system, which forces the movement of the liquid to balance the pressure in the system. It should be noted that the sample source (50,60,62) and the waste channel (61), connected to the cylinder (1), are open to the outside environment or are under a protective atmosphere. Unlike the solution in variant I, described above, the reservoirs (20) are sealed. The area of influence of the generated pressure changes in the hydraulic system, understood as the whole device (including channels, cartridge reservoirs, connectors and hoses) connected to the cylinder (1), is limited by the position of the pistons (2) that can move freely inside the cylinder (1). The fluid flow in the system is thus controlled mainly hydraulically. Changing the relative position of the pistons (2) in the cylinder (1) generates the aforementioned pressure changes in the hydraulic system, however, unlike variant I, according to the invention, variant II does not allow refilling or reducing the amount of gas inside the system. In case there is one of the holes (10,12,14,16) in the working space between the pistons (2), the change in the position of the pistons (2) generates the movement of the liquid in the direction that balances the pressure changes, i.e. when the pistons (2) are moved apart, the liquid is sucked into the cylinder (1), and when the pistons (2) are pushed back, the liquid is pushed out of the cylinder (1). It is worth noting that when the pistons (2) move in the cylinder (1) in the same direction with the same speed, the so-called travel in a hydraulically and pneumatically neutral mode, which does not generate the pumping of fluids through the holes (10,12,14,16) and allows the reaction solution to be moved to the desired area of the cylinder (1), for example, to draw a specific reagent, pump the solution to the mixer (20D), pump the solution to the area of the detection chamber (6) or remove the solution from the cylinder (1) to the waste channel (61).

Conducting the specific reaction and subsequent optical determination of its product requires precise pumping of fluids in the hydraulic system, which in turn requires precise control of the position of the pistons (2) in the cylinder (1) and precise control of fluid levels in reservoirs (20).

According to the invention, the position of the pistons (2) is controlled by the electronic main controller (88), controlling the entire device, via the stepper motors (3) equipped with lead screws and positioning nuts connected to the connectors (4) of the pistons (2). The number of steps of the stepper motors (3) and the thread pitch of the lead screws directly determine the precision and resolution of the movement of the pistons (2), which in turn translates into the precision of the control over the volume of the working space of the cylinder (1) between the pistons (2), available for the reaction solution. It is possible to use any stepper motors (3) with any lead screws and positioning nuts, preferably the same for both pistons (2). Due to the ease of calculation and simulation of the position of the pistons (2), stepper motors (3) with the number of steps of 200/3600, equipped with lead screws with a thread pitch of 1 mm with compatible positioning nuts, are preferably used. Such parameters of the set of stepper motors (3), applied to a preferred hydraulic system with a cylinder (1) with a diameter of 6 mm, results in the ability to manage the fluids with an accuracy of 141 nl, which is about 2% of the minimum volume of the tested solution necessary for one-time dosing, which is 7 μl, and ensures sufficiently precise control of the system. The use of a stepper motor (3) with a larger number of steps and a lead screw with a smaller pitch allows for increasing the resolution of the control of the working volume of the cylinder system (1).

The process of drawing the fluids from the reservoirs (20) and the sample source (50,60,62) according to the invention is generated by creating a vacuum in the cylinder (1) in the area of the hole (10) and the hole (12), respectively. The fluids flowing out of the holes (10,12) fill the space between the pistons (2) of the cylinder (1) and possibly combine with the solution filling the cylinder (1). In order to avoid uncontrolled outflow of liquids from the holes (10) and the contamination of the reagents in the reservoirs (20), the reservoirs (20) containing the reagents have a movable piston (21), the movement of which allows the volume of the reservoirs (20) to be reduced under the influence of the negative pressure applied by the pistons (2) moving in the cylinder (1). The neutral movement of the pistons (2) does not cause pressure changes in the reservoirs (20). Contamination of the reagents is also avoided by using a removable membrane on the pins (25) of the cartridge (30), e.g. in the form of a peelable foil which is removed before the cartridge (30) is docked to the device.

In order to maintain the repeatability of the process of drawing the liquids from the reservoirs (20), before starting the measurement cycle, during the preparatory stage, after the cartridge (30) docking, liquids are sequentially drawn from each of the reservoirs (20A,20B,20C), and then the cylinder (1) is rinsed several times with a fresh portion of the currently tested sample. This process allows to standardise the filling of the channels (10,11,28,25,24,22) in the system. Precise control of the volume of the fluid drawn is possible thanks to the certainty that the channels (10,11,28,25,24,22) are always filled to the same extent.

After the reagents, necessary to carry out the specific reaction, are taken to the cylinder (1) and pumped into the mixer (20D), the reaction solution is preferably mixed by pumping it several times between the mixer (20D) and the cylinder (1). During serial determinations, a specific, always the same, amount of time must be allowed before the determination to ensure uniform intervals between the determinations. When determining this time, the time needed to pump the solution from the sample source (50,60,62) and the time needed to clean the cylinder (1), the reservoir (20D) and the detection chamber (6) should also be taken into account.

Before the determination, a portion of the sample with a total volume of 6-12 ml, preferably 8 ml, is taken several times (at least 3 times, preferably 4 times) to wash the cylinder (1) to prepare it for the reaction, and additionally wash the detection chamber (6).

It is essential that during the determination the detection chamber (6) is filled with the reaction solution to such an extent that the optical path (72,73) is completely covered by the solution to be determined. The reaction time is determined by the operator based on specific analytical needs and the characteristics of the specific reaction being conducted. When monitoring changes in the level of the analyte in the solution stream, it is important to ensure the appropriate sampling frequency. For processes lasting 2-5 hours, it is preferable to sample every 5-15 minutes. The reaction time should not exceed half of the sampling period to ensure that the cylinder (1) can be washed before the next determination.

During the determination, appropriate solutions are mixed together: the tested sample (or matrix solution, or standard solution during the reference measurements) and at least one reagent, two reagents, or more reagents, depending on the test. The total volume of the reaction solution is set to achieve the appropriate volume of the reaction solution to fill the detection chamber (6) and ensure full coverage of the optical path (72,73) along its entire width, which is 1-10 mm, preferably 4 mm. Only then, the analytical signal is maximised and the measurement process becomes repeatable. When using a detection chamber (6) with a volume in the range of 35-400 μl, preferably 63 μl, a reaction solution of at least three times the volume, preferably 240 μl, is prepared to ensure that the detection chamber (6) is rinsed and completely filled with the tested solution. Preferably, depending on the assay, the volume of the tested sample equals 30-90 μl, the volume of reagents used equals 50-250 μl, and the resulting reaction mixture has a volume of 240-320 μl. Preferably, the volume of the reaction solution is minimised, which facilitates the manipulation of solutions, speeds up the measurement and reduces the volume of collected reagents.

### The Special Use Case of the Device to Monitor the Progress of the Blood Dialysis Treatment

The device in the version for monitoring the progress of blood dialysis process by tracking the changes in the concentration of uremic toxins in the post-dialysis fluid stream, according to the invention, is adapted to determine creatinine, urea and phosphate ions in the dialysate (i.e. the main uremic toxins), which are determined by specific chemical reactions.

The device adapted for the analysis of post-dialysis fluid is connected to the waste channel (60) of the haemodialysis machine through the sampling system with the hose (41) which uptakes successive portions of the post-dialysis fluid are directly from the waste stream of the haemodialysis machine. Due to the need of keeping the haemodialysis machine sterile, the device is connected to the haemodialysis machine indirectly through the airlock (50), which task is to physically break the continuity of the waste channel (60) of the haemodialysis machine upstream to the sampling point with the hose (41), thanks to which the contamination of the haemodialysis machine is impossible with microorganisms migrating along the walls of the waste channel upstream from the sampling point to the haemodialysis machine. The use of the airlock (50) increases the safety of the patients undergoing blood dialysis and eliminates the need of disinfection of the device according to the invention between the successive patients.

Preferably, the device has three optical detection systems (70) in a separable (FIG. 10) or cross (FIG. 11) configuration containing three different light sources (71) emitting monochromatic light with a wavelength adapted to three analytes, or most preferably the device has an optical detection system (70) with an integrated SMD diode equipped with three different light sources (71) emitting monochromatic light with a wavelength adapted to three analytes (FIG. 29, FIG. 48). Light sources (71) emitting monochromatic radiation are used for the determination of uremic toxins:
to determine the product of reaction of creatinine: 500-550 nm, preferably 525 nm,
to determine the product of reaction of urea: 410-460 nm, preferably 415 nm,
to determine the product of reaction of phosphate ions: 550-900 nm, preferably 625 nm.

However, it is possible to use the optical detection system (70) in other configurations described, i.e. comprising a light source (71) emitting light with an adjustable wavelength or emitting white radiation with a continuous spectrum.

According to the invention, before monitoring the progress of blood dialysis, the analyte (toxin) for determination is selected from: creatinine, urea and phosphate ions, and then the analyte standard solution is placed in the reservoir (20A/95A), and chemical reagents stored in the reservoirs (20B/95B, 20C/95C) for carrying out the specific reaction. Any specific reaction may be used according to the invention, but the following reactions are considered preferred.

Creatinine, according to the invention, is determined using Jaffe method. The reaction between creatinine and picric acid (2,4,6-trinitrophenol) in alkaline conditions is carried out according to the following reaction scheme [Anal. Methods 7 (2015), 6904-6910]:

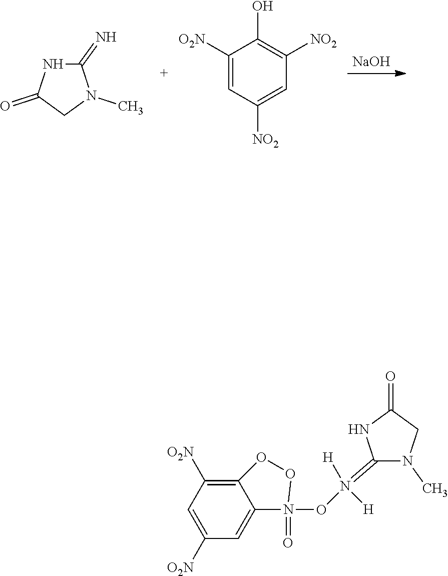

The product of the reaction is an orange-red adduct of creatinine with picric acid, which can be determined spectrophotometrically at the wavelength range of 470-550 nm. According to the invention, the light source (71) emitting light with a wavelength in the range of 500-550 nm, preferably 525 nm, is used to determine the concentration of this adduct. A diode with a wavelength of 525 nm is used as the detector (74), while a diode of 625 nm is used as the detector (75). When conducting the specific reaction, according to the invention, the following reagents are used to fill the reservoirs (20/95) in the cartridge (30/90) with the standard solution (ST), reagents (R1,R2) and possibly the matrix solution (MATRIX) or left empty as a mixer (MIX):
(20A/95A): ST: aqueous solution containing: 583 μM creatinine [CAS: 60-27-5]
(20B/95B): R1: aqueous solution containing: 25 mM picric acid [CAS: 88-89-1]
(20C/95C): R2: aqueous solution containing: 200 mM sodium hydroxide [CAS: 1310-73-2]
(95D): MATRIX: pure dialysis fluid sampled before the start of blood dialysis process
(20D): MIX: empty reservoir to be used as a mixer

The sample contains creatinine (analyte). Range of determination: 17-530 μM.

Proportions of the reaction mixture: dialysate sample (100/350)+R1 (100/350)+R2 (150/350)

Urea, according to the invention, is determined using the photometric method employing Ehrlich's reagent. The reaction between urea and 4-(dimethylamino)benzaldehyde (DMAB) is carried out in acidic conditions. The reaction is initiated by protonation of the dimethylamino group, which enables nucleophilic attack by the nitrogen of the urea amino group. A coloured adduct is formed according to the reaction scheme [J. Chil. Chem. Soc. 62 (2017) 3538-3542]:

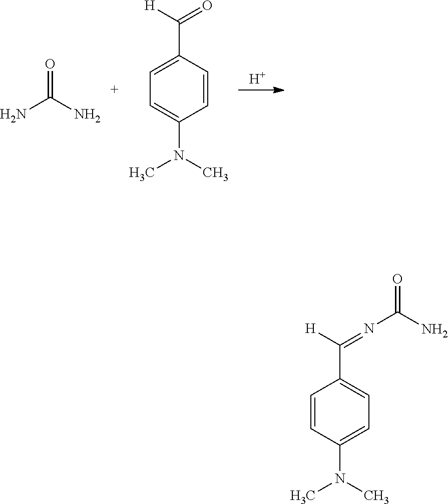

The product of the reaction is an adduct of urea with DMAB, determined spectrophotometrically at the wavelength range of 410-460 nm. To determine the concentration of this adduct, a light source (71) emitting light with a wavelength in the range of 410-460 nm, preferably 415 nm, is used. A 460 nm diode is used as a detector (74) and a 625 nm diode is used as a detector (75). When conducting the specific reaction, according to the invention, the following reagents are used to fill the reservoirs (20/95) in the cartridge (30/90) with the standard solution (ST), reagents (R1,R2) and possibly the matrix solution (MATRIX) or left empty as a mixer (MIX):
(20A/95A): ST: aqueous solution containing: 16 mM urea [CAS: 54-13-6]
(20B/95B): R1: water/ethanol solution containing:
   120 mM 4-(dimethylamine)benzaldehyde [CAS: 100-10-7]
   78.5% v/v ethanol [CAS: 64-17-5]
   72 mM hydrochloric acid [CAS: 7647-01-0]
(20C/95C): R2: aqueous solution containing: 400 mM hydrochloric acid [CAS: 7647-01-0]
(95D): MATRIX: pure dialysis fluid sampled before the start of blood dialysis process
(20D): MIX: empty reservoir to be used as a mixer
The sample contains urea (analyte). Range of determination: 0.2-16 mM
Proportions of the reaction mixture: dialysate sample (125/440)+R1 (200/440)+R2 (115/440)

Phosphate ions are determined by the phosphomolybdenate method. The molybdenum blue formation reaction takes place in two stages. The phosphomolybdenate method requires the use of a strong acid, a source of Mo(VI) and a reducing agent, usually in an aqueous solution. The first stage results in the formation of a Keggin structure around the analyte ion, while in the second stage the heteropolyacid formed in the first stage is reduced to a dark blue product according to the reaction scheme [Analytica Chimica Acta 896 (2015) 120-127]:

$${{PO}_{4}^{3 -} + 12 {MoO}_{4}^{2 -} + 27 {H}^{+} \to {( {H}_{3} {PO}_{4} ( MoO )}_{3} )}_{12} + 12 {H}_{2} O$$

$${H}_{3} {PMo}_{12}^{( VI )} + reduktor \to {[ {H}_{4} {PMo}_{8}^{( VI )} {Mo}_{4}^{( V )} {O}_{40} ]}^{3 -}$$

The product of the reaction is molybdenum blue, which can be determined spectrophotometrically at the wavelength range of 550-900 nm. According to the invention, a light source (71) emitting light with a wavelength in the range of 550-900 nm, preferably 625 nm, is used to determine the concentration of this adduct. Diodes with a wavelength of 625 nm are used as a detector (74) and a detector (75). When conducting the specific reaction, according to the invention, the following reagents are used to fill the reservoirs (20/95) in the cartridge (30/90) with the standard solution (ST), reagents (R1,R2) and possibly the matrix solution (MATRIX) or left empty as a mixer (MIX):
(20A/95A): ST: aqueous solution containing: 1000 μM sodium phosphate [CAS:7601-54-9]
(20B/95B): R1: aqueous solution containing:
20 mM ammonium ortomolibdenate [CAS: 236-031-3]
2.10 mM antimony potassium tartrate [CAS: 28300-74-5]
800 mM sulphuric(VI) acid [CAS: 76664-93-9]
(20C/95C): R2: aqueous solution containing: 120 mM ascorbic acid [CAS: 50-81-7]
(95D): MATRIX: pure dialysis fluid sampled before the start of blood dialysis process
(20D): MIX: empty reservoir to be used as a mixer
The sample contains phosphate ions (analyte). Range of determination: 0.2-800 μM
Proportions of the reaction mixture: dialysate sample (50/450)+R1 (100/450)+R2 (300/450)

The concentrations were optimised taking into account the range of the expected analyte concentration. When monitoring changes in the concentration of toxins in the dialysate, the maximum concentrations (initial, pathological) are usually ten times higher than the minimum (final, normative) concentrations of uremic toxins in the tested samples. Depending on the equipment used during blood dialysis, maximum and normative concentrations may vary, but they are correlated with pathological and physiological concentrations of these toxins in body fluids. Nevertheless, for each device these dependencies are determined experimentally. Physiological concentrations of uremic toxins in the blood (plasma) are usually at the levels described in the literature, i.e. creatinine: 53-115 μmol/l (adults); urea: 2.5-6.7 mmol/l; phosphate ions (so-called inorganic phosphorus): 810-1620 μmol/l. Pathological concentrations of uremic toxins, observed in patients with renal failure, reach much higher levels (Table 1).

TABLE 1
Maximum pathological concentrations of uremic toxins
were experimentally confirmed during the numerous determinations
of dialysate and serum samples tested.

| toxin | dialysate | | | | serum | | | |
|---|---|---|---|---|---|---|---|---|
| creat- | 6.24 | mg/dL | 552 | μmol/L | 15.18 | mg/dL | 1265 | μmol/L |
| inine | | | | | | | | |
| urea | 146.7 | mg/dL | 24.47 | mmol/L | 233.7 | mg/dL | 38.95 | mmol/L |
| phos- | 2.93 | mg/dL | 950 | μmol/L | 10.38 | mg/dL | 3350 | μmol/L |
| phate | | | | | | | | |
| ions | | | | | | | | |

A normal cartridge (90) filled with reagents is inserted into the device. Matrix solution (pure dialysis fluid) is either placed in the reservoir (95D) prior to the insertion of cartridge (90) into the device, or sampled from the waste stream (60) of the haemodialysis machine prior to the initiation of actual blood dialysis and is kept for use at further stage of the monitoring of the dialysis process. Preferably, an inverse cartridge (30) filled with reagents is inserted into the device. The matrix solution (pure dialysis fluid) is sampled from the waste stream (60) of the haemodialysis machine prior to the start of the actual blood dialysis for the initial calibration measurement and is used at this stage of the monitoring of dialysis process, leaving the tank (20D) empty to be used as a mixer.

According to the invention, it is possible to use any specific reactions for the determination of uremic toxins, but the above-described reactions are considered to be particularly advantageous.

Before starting the monitoring of dialysate, calibration measurements are carried out using a standard solution and a matrix solution (two-point calibration). It is possible to carry out a quick, two-point calibration as well as a multi-point calibration, thanks to the possibility of automatic preparation of a concentration series of a standard solution diluted with a matrix solution. Most preferably, full multi-point calibrations are performed before and after the dialysis, and single-point calibrations are performed during the dialysis as needed.

While monitoring the progress of blood dialysis, the post-dialysis fluid is sampled at regular intervals, for example every 5-15 minutes, and the temporary concentration of the selected analyte in the dialysate stream is determined. Saving the results of subsequent determinations in a series, makes possible to create a dynamic curve of the decrease of the concentration of the toxin in the dialysate as a function of the dialysis duration, and the results are observed in real time on an external device with the option of archiving or printing them.

In the prior art, dialysis is routinely performed using standard doses of the dialysis fluid in a process of a standard length. Determination of the blood dialysis effectiveness is carried out by analysing patient's blood samples taken before and after the dialysis, the results of which are not known until the following day. Therefore, there is no possibility of faster and safe termination of the blood dialysis treatment nor the method of detecting its irregularities, which result in the inconveniencies for the patients which are associated with the long duration of the blood dialysis procedure and often the need of an urgent repetition of the treatment.

The solution according to the invention constitutes a significant innovation in blood dialysis monitoring. The analysis of the changes of the concentration of toxin in the dialysate as a function of time, the effectiveness and correctness of the of the dialysis process can be concluded. When the toxin level, successively decreasing, reaches the normative level that would be observed during dialysis of a healthy person, indicating the effective purification of the patient's blood, it is possible and suggested to terminate the dialysis, because further treatment has no medical sense, unnecessarily exposing the patient to the inconvenience of dialysis, and unnecessarily blocking the haemodialysis machine, which could be used for dialysis of another patient. According to the invention, the end of dialysis is determined taking into account its dynamics, which means that the treatment can be shorter than standard, but also longer than standard, if necessary. The alarm system (80) is then activated, informing about the possibility of safe termination of the dialysis. In turn, when the toxin level behaves abnormally, an alarm system (80) is activated to inform about possible errors in the dialysis process. This is especially important because a sudden drop in toxin concentration indicates that the patient's blood has not been cleared despite ongoing dialysis.

Between successive samplings and determinations of the analyte, the cylinder (1) is washed with a portion of the dialysate stream of the current composition, while using variant I of the device, the matrix solution from the reservoir (95D) can be used for this purpose. Preferably, the cylinder (1) is washed three times.

It is possible to conduct one-point calibration measurements between successive samples and determinations of the analyte using the standard solution from the reservoir (20A/95A), while using variant I of the device, it is possible to conduct two-point calibration using additionally the matrix solution from the reservoir (95D).

The device apparatus for the automated determination of an analyte in the liquid phase and a method for the automated determination of an analyte in the liquid phase using this apparatus, in particular for monitoring the progress of the blood dialysis process, according to the invention, are described below in examples of embodiment.

### EXAMPLES

Example 1. A device for the automated determination of an analyte in the liquid phase, according to the invention, was manufactured, in variant I, with a horizontally oriented cylinder (1) embedded in the device, which model and a functional diagram are shown in FIG. 1, FIG. 3, FIG. 5A. As the cylinder (1), a commercially available, disposable, medically certified PP syringe with an internal diameter of 12 mm and a volume of 5 ml was used, which was devoid its the bottom. As pistons (2), two-piece medically certified PP pistons made of were used, constituting a commercially available set with the type of syringe used. Seven holes with a diameter of 1.3 mm were drilled in the wall of the syringe (1): alternating four (10) on one side and three (12,14,16) on the opposite side, while the distance between the projections of these holes was 5 mm, in the sequence from left to right: 10A, 12, 10B, 14, 10C, 16, 10D. PTFE hoses (99,41,47,44) with 0.8 mm internal diameter and 0.25 mm wall thickness were inserted into these openings and sealed with 1.110 mm thick waterproof flexible double-sided foamed adhesive tape. Four hoses (99) with a length of 16 cm were embedded in holes (10) as well as in through in tips (98) made of conductive PP doped with carbon particles, rigidly mounted from the bottom in sockets (97) located in the upper plane of the bed for the cartridge. Tips (98) had a length of 76 mm and a diameter of 6 mm, which decreased downwards, with the inside diameter of the lower edge being 1.3 mm, and the lower edge of the hoses (99) tangential to the lower edge of the tips (98). One hose (47) 4 cm long was placed in the hole (14) and its outlet was directed upwards. 10 cm long hoses (41,44) were inserted into the holes (12,16) and their other ends were thermally curved and placed in the drain pipe (61) of the haemodialysis machine, on which the airlock (50) was located as in FIG. 56B. Three optical detection systems (70) were placed around the cylinder (1), each containing a light source (71) in the form of a diode and two diode detectors (74,75), shown in FIG. 10. One of the optical detection systems (70) contained green diodes (LL-504PGC2E-G5-1AC) with a wavelength of 525 nm as an emitter (71) and a detector (74) for the determination of creatinine acid adduct, while the second contained a violet diode (OSV6YL5111A) with a wavelength of 415 nm as an emitter (71) and a violet diode (OSB44P5161A) with a wavelength of 460 nm as a detector (74) for the determination of urea adduct with DMAB, while the third contained red diodes (LL-504PGC2E-G5-1AC) with a wavelength of 625 nm as an emitter (71) and a detector (74) for determination of molybdenum blue. Each of the systems (70) contained a red diode (LL-504PGC2E-G5-1AC) with a wavelength of 625 nm (or other wavelength, e.g. 610 nm, 640 nm) as a universal fluorimetric/nephelometric detector (75). The pistons (2) were connected via connectors (4) and trapezoidal positioning nuts to two identical stepper motors (3), with a step of 200/3600, with trapezoidal lead screws, parallel, misaligned with the pistons (2). The device is equipped with guides (93) and a lift (94) controlled by the ARDUINO controller (88). In the bed of the device, a normal cartridge (90) with an openwork frame cooperating with the guides (93) and the lift (94) was placed. The cartridge (90) contained four reservoirs (95) in the form of commercially available conical bottom eppendorf centrifuge vials with a volume of 25 ml and a height of 78 mm. Cups (96) had axially located hole closed with a 14 mm septum membrane. The device was equipped with an alarm system (80), equipped with a speaker (81), a multi-LED light source (82) with variable colour and means of remote communication (83). All the structural elements that kept the above-mentioned functional elements in the presented orientation were made of a PLA filament with the 3D printing technique using CREATOR 3 Flashforge printer.

Example 2. A device for the automated determination of an analyte in the liquid phase was manufactured as in Example 1, in variant I, with the difference that a two-piece normal cartridge (90) shown in FIG. 2 was used. A PET cylinder (1) with a diameter of 10 mm and one-piece pistons (2) of PET with a diameter of 10 mm were used. An optical detection system (70) with a cross arrangement of diodes and detectors was used (FIG. 11). The RASPBERRY PI controller (88) was used. Structural elements keeping the functional elements in the given orientation were manufactured form ABS using 3D printing technology.

Example 3. A device for the automated determination of an analyte in the liquid phase was manufactured as in Example 1, in variant I, with the difference that a cylinder (1) in the form of a PET syringe with an internal diameter of 14 mm and matching one-piece PET pistons (2) were used. One optical detection system (70) with a light source of adjustable wavelength in the form of a white light emitter equipped with a monochromator was used (FIG. 8).

Example 4. A device for the automated determination of the analyte in the liquid phase was manufactured as in Example 1, in variant I, with the difference that a cylinder (1) in the form of a PET syringe with an internal diameter of 14 mm and matching one-piece pistons (2) were used. from PET. One optical detection system (70) with a source of white light with a continuous spectrum was used (FIG. 9).

Example 5. A device for the automated determination of the analyte in the liquid phase was manufactured as in Example 1, in variant I, with the difference that the cartridge (90) contained an embedded cylinder (1) in a horizontal orientation and also tips (98). Some of the structural elements keeping the functional elements in the given orientation, manufactured form PLA using 3D printing technology, had a different shape.

Example 6. A device for the automated determination of an analyte in the liquid phase, according to the invention in variant I, was manufactured with a vertically oriented cylinder (1) embedded in the device, the functional diagram of which is shown in FIG. 4, FIG. 5B. The device consisted of the same elements as the device in Example 1, with the difference that the cylinder (1) was oriented vertically and the sequence of holes from top to bottom was: 12, 14, 10A, 14, 10B, 14, 10C, 14, 10D, 14, 16. Some of the construction elements made in the 3D printing technique using PLA had a different shape, keeping the functional elements in the correct orientation.

Example 7. A device prepared in Example 1 was tested to determine changes in creatinine concentration in the dialysate stream. Preliminary photometric tests of the product of Jaffe reaction between creatinine and picric acid were carried out using a conventional spectrophotometer. Absorption spectrum was recorded and a calibration curve was constructed at 525 nm with a linear response in the range of 17-530 μM (FIG. 12). Then, comparative measurements of the changes in creatinine concentration in the dialysate stream were performed. Measurements using a classical spectrophotometer and quartz cuvettes were performed manually, sampling the dialysate stream every 15 minutes (FIG. 13A). Automated measurements at a frequency of 15 minutes were performed using the device made in Example 1, having an aqueous solution containing 25 mM picric acid as the first reagent and an aqueous solution containing: 200 mM sodium hydroxide as the second reagent (FIG. 13B). The same proportions of the reaction mixture were used each time: dialysate sample (100/350)+R1 (100/350)+R2 (150/350). Automated measurements were preceded by drawing pure dialysis fluid from an external reservoir, which was the waste channel of the haemodialysis machine connected to the device through an airlock. Prior to the measurement of the first portion of the dialysate, a multi-point calibration was performed using the standard solution (583 μM creatinine) and the matrix solution (pure dialysis fluid). No significant differences were observed between the classical and automated measurement according to the invention.

Example 8. The device produced in Example 1 was tested to determine changes in urea concentration in the dialysate stream. Preliminary photometric tests of the product of the reaction using Ehrlich's reagent between urea and p-N,N-dimethylaminobenzaldehyde, employing a conventional spectrophotometer. Absorption spectrum was recorded and a calibration curve was constructed at 415 nm with a linear response in the range of 0.2-16 mM (FIG. 14). Then, comparative measurements of the changes in creatinine concentration in the dialysate stream were performed. Measurements using a classical spectrophotometer and quartz cuvettes were performed manually, sampling the dialysate stream every 15 minutes (FIG. 15A). Automated measurements at a frequency of 15 minutes were performed using the device made in Example 1, having a water/ethanol solution containing 120 mM DMAB, 78.5% v/v ethanol and 72 mM hydrochloric acid as the first reagent and an aqueous solution containing 400 mM hydrochloric acid as the second reagent (FIG. 15B). Automated measurements were preceded by drawing pure dialysis fluid from an external reservoir, which was the waste channel of the haemodialysis machine connected to the device through an airlock. Prior to the measurement of the first portion of the dialysate, a multi-point calibration was performed using the standard solution (16 mM urea) and the matrix solution (pure dialysis fluid). The same proportions of the reaction mixture were used each time: dialysate sample (125/440)+O1 (200/440)+O2 (115/440). No significant differences were observed between the classical and automated measurement according to the invention.

Example 9. The device produced in Example 1 was tested to determine changes in phosphate ions concentration in the dialysate stream. Preliminary photometric tests of the product of the two-step phosphomolybdenum method involving a reaction between phosphates and molybdates in acidic conditions to form a Keggin structure, which is then reduced to molybdenum blue. Absorption spectrum was recorded and a calibration curve was constructed at 625 nm with a linear response in the range of 0.2-800 μM (FIG. 16). Then, comparative measurements of the changes in creatinine concentration in the dialysate stream were performed. Measurements using a classical spectrophotometer and quartz cuvettes were performed manually, sampling the dialysate stream every 15 minutes (FIG. 17A). Automated measurements at a frequency of 15 minutes were performed using the device made in Example 1, having a water solution containing 20 mM ammonium orthomolybdate, 2.10 mM potassium antimonyl tartrate and 800 mM sulfuric acid as the first reagent and an aqueous solution containing 120 mM ascorbic acid as the second reagent (FIG. 17B). Automated measurements were preceded by drawing pure dialysis fluid from an external reservoir, which was the waste channel of the haemodialysis machine connected to the device through an airlock. Prior to the measurement of the first portion of the dialysate, a multi-point calibration was performed using the standard solution (1000 μM sodium phosphate) and the matrix solution (pure dialysis fluid). The same proportions of the reaction mixture were used each time: dialysate sample (50/450)+O1 (100/450)+O2 (300/450). No significant differences were observed between the classical and automated measurement according to the invention

Example 10. A device for the automated determination of an analyte in the liquid phase, according to the invention, was manufactured, in variant I, with a horizontally oriented cylinder (1) embedded in the device, the model and functional diagram of which are shown in FIG. 18, FIG. 19 and FIG. 20, and its functional elements are shown in FIG. 22, FIG. 23, FIG. 24, FIG. 29, FIG. 30, FIG. 33 and FIG. 56.

The housing block (5) with a cuboid shape, 94 mm long, 28 mm wide and 33 mm high, was made by classical machining techniques using PEEK. In the housing block (5), along its longitudinal axis, perpendicularly to its side walls, a through opening with an internal diameter of 10 mm was drilled for the cylinder (1) made of a plastic material. In the housing block (5), a transverse, horizontal, circular opening for the detection chamber (6) was drilled, with a diameter of 4 mm, perpendicular to the front wall of the housing block (5) and perpendicular to the opening for the cylinder (1), which axes crossed in the area of their intersection. On the front wall and rear wall of the housing block (5), around the thorough opening for the detection chamber (6), two M2 threaded holes, 5 mm deep, were drilled for mounting screws of the elements of the optical detection system (70), located in opposite corners of a square with a side length of 8.5 mm, which geometrical centre was the axis of the detection chamber (6). A PMMA rod with an outer diameter of 10 mm and a length of 94 mm, covered with acrylic glue, was placed in the through opening of the cylinder (1), and let to dry. Then, in the housing block (5), perpendicularly to its base, in a vertical plane passing through the axis of the cylinder (1), channels (11,13,15,17) with an internal diameter of 1 mm were drilled, with four channels (11) from the top of the housing block (5), and channels (13,15,17) from the bottom. The channels were drilled in a sequence of 10A-12-10B-14-10C-16-10D of the projections of the channels' axes on the axis of the cylinder (1) with a spacing of 11 mm of these projections. The channels (11,13,15,17) were drilled in such a way that they did not went through the acrylic rod, but only reached its axis. Then, a through hole with an internal diameter of 6 mm was drilled in the axis of the acrylic rod, creating an acrylic cylinder (1) with an internal diameter of 6 mm and a wall thickness of 2 mm, equipped with holes (10,12,14,16) in its walls, opening the previously drilled channels (11,13,15,17), respectively, with an 11 mm spacing between their axes on the axis of the cylinder (1) and the sequence 10A-12-10B-14-10C-16-10D. As the pistons (2) of the cylinder (1), a two-piece system was used, consisting of a PTFE piston rod with a diameter of 6.2 mm and a length of 30 mm, with a brass guid holder embedded, further connected through connectors (4), with the nuts on the lead screws (Tr 8×1) of the stepper motors (3). From the bottom of the housing block (5), four M4 threaded through holes were drilled for mounting on the device's supporting plate, located in the corners of a rectangle measuring 53×20 mm, with the geometrical centre in the axis of the channel (15). From the top of the housing block (5), at the exit of each of the four channels (11), ports (28) with a diameter of 6.2 mm and a depth of 17.2 mm were drilled, with a stepped undercut (29) with a diameter of 10.25 mm and a depth of 5 mm, receiving a gasket (27) in the shape of an O-RING with a diameter D/d of 6/2 mm. From the top of the housing block, between the ports (28A,28B) and (28C,28D), M4 threaded holes with a depth of 8 mm were drilled, receiving pillars (38) positioning the cartridge (30) relative to the housing block (5). From the top of the housing block (5), on its transverse axis, at a spacing of 21.8 mm, M3 threaded holes with a depth of 5.3 mm were drilled, located at the bottom of square depressions with a depth of 3.2 mm and a side length of 8 mm, centred relative to the axis of the M3 holes, receiving screws stabilising the position of the lid (26) in the vertical axis. From the bottom of the housing block (5), at the outlet of the channels (13,15,17), in the 3 mm thick protrusions, R1/8 threaded holes 7 mm depth were drilled, accepting FESTO/SMC quick connect fittings. The housing block (5) in this variant is shown in FIG. 20, FIG. 22 and FIG. 24.

The compression lid (26) 94 mm long and 28 mm wide was made of PEEK using machining techniques. In the lid (26) through holes with an internal diameter of 6.2 mm and a height of 12.7 mm were milled, the wall thickness of the lid (26) was 2.5 mm, and the through holes protruded beyond the outline of the lid's plane at 6 mm from the top and 4.2 mm from the bottom. In the lid (26), on its transverse axis, at a spacing of 21.8 mm, through holes with a diameter of 3.5 mm were drilled, located in a square depression with a side length of 7 mm, protruding from the bottom beyond the plane of the lid (26) by 2.5 mm, so that the heads of the stabilising screws did not extend beyond the plane of the lid (26). The lid (26) in this variant is shown in FIG. 20, FIG. 22 and FIG. 24.

The optical detection system (70) was set with a light source (71) in the form of a integrated LED SMD diode, LUMIXTAR WL-1.5P5054EP120C3b1-RGV, and one universal detector (74) in the form of a CCD matrix with an RGB filter, HAMAMATSU S13683, with 40 zones, four channels, operating in RGB channels and a correction channel. The light source (71) was capable of emitting monochromatic light at three wavelengths: 525 nm (for the determination of creatinine), 415 nm (for the determination of urea) and 625 nm (for the determination of phosphate ions). The emitter (71) was placed on a 32×12 mm mounting plate with through holes for M2 mounting screws, compatible with the mounting holes on the side walls of the housing block (5). The emitter (71) was mounted on the housing block (5) and centred on the axis of the detection chamber (6). The detector (74) was placed on a 32×12 mm mounting plate with through holes for M2 mounting screws, compatible with the mounting holes on the side walls of the housing block (5). The detector (74) was mounted on the housing block (5), on its other side in relation to the light source (71), on the axis of its optical path (72), and centred on the axis of the detection chamber (6). The optical detection system (70) in this variant is shown in FIG. 12 and FIG. 13.

The cartridge (30), 136 mm long, 41 mm wide and 95 mm high, with a cuboidal body of dimensions of 100×41×95 mm, with a wall thickness of 2 mm, equipped with side protrusions forming sockets (34), was made of ABS by injection technique. The sockets (34) on the two side walls of the cartridge housing (30) receiving the forks (35) of the lift (36) of the device were 4 mm wide and 27 mm high, and had a form of through undercuts on the inner planes of the side protrusions. The cartridge (30) consisted of a housing (31) and a lid (32), which were joined at the middle of the cartridge (30) by a one-time snap lock (33). The housing (31) (lower part) had LUER sockets (23) on the lower inner surface, receiving the LUER dispensing tips (22) of four reservoirs (20A,20B,20C,20D) in the form of syringes with a capacity of 10 ml each. The sockets (23) were connected by channels (24) of an internal diameter of 1 mm with through pins (25) extending beyond the lower outer surface of the cartridge (30), compatible with the ports (28) at the outlet of the channels (11) in the outer surface of the housing block (5) of the cylinder (1). The through pins (25) had an inner diameter of 1 mm, an outer diameter of 6 mm and a height of 16 mm. The outlet of each of the through pins (25A,25B,25C,25D) was secured from the bottom with a tear-off plate, protecting the content of the cartridge (30) against contamination. The cartridge (30) was equipped with an electronic circuit with non-volatile RFID NTAG213 memory with an 8×18 mm antenna, on an 11×21 mm adhesive backing, which was placed in a dedicated internal niche in the lower part of the housing (31). The cartridge (30) in this variant is shown in FIG. 22 and FIG. 23.

The housing block (5) was mounted on the device's supporting plate with 17 mm long M4 screws, fixed in the through-holes of the housing block (5). From the bottom, in the sockets at the outlet of the channels (13,15,17), FESTO/SMC quick connect fittings were placed, to which were plugged the hoses (41,44,46) connecting the device with the airlock (50) and the waste channel (61). Positioning pillars (38) with a diameter of 4.85 mm and a length of 25.6 mm, equipped with an M4 thread of a length of 6.20 mm, were mounted on the housing block. O-RING seals with a diameter of D/d 6/2 mm were placed in the ports (28). A lid (26) was put on the housing block and fixed with 8 mm M3 screws. Pistons (2) were inserted into the cylinder (1) in the housing block (5). Drive units with stepper motors (3) were mounted on the supporting plate and connected to the pistons (2) by connectors (4). A lift (36) was assembled with forks (35) on the vertical guiding pillars and connected to a drive equipped with a dedicated stepper motor. The whole setup was placed in a dedicated housing. The electronic main controller (88), equipped with antennas for remote communication, in a compact form and DIN standard housing was placed in a separate compartment in the back of the device. The electronic main controller (88) was connected with dedicated wires to the drive system of the pistons (2), the drive system of the lift (36), the RFID antenna, the optical detection system (70) and the airlock (50). A cartridge (30) filled with a set of reagents dedicated to determination of creatinine (Example 12) was taken, the protective tear-off plates were removed from its through pins (25A,25B,25C,25D) and placed on the fork (35) of the lift (36). The device prepared in this way was ready to monitor the concentration of creatinine in the dialysate.

Example 11. A device for the automated determination of an analyte in the liquid phase according to the invention was manufactured, in variant II, with a horizontally oriented cylinder (1) mounted as in example 10, with the difference that a two-element housing block (5) was made for the cylinder (1) made of quartz. The functional diagram of this variant is shown in FIG. 21, and its functional elements are shown in FIG. 25.

The housing block (5) with a cuboid shape, 94 mm long, 28 mm wide and 27 mm high, was made of aluminium using classical machining techniques. In the housing block (5), along its longitudinal axis, perpendicular to its side walls, a through hole was drilled with an inside diameter of 12 mm to embed the cylinder (1). In the housing block (5), a transverse, horizontal, circular opening for the detection chamber (6) was drilled, with a diameter of 4 mm, perpendicular to the front wall of the housing block (5) and perpendicular to the opening for the cylinder (1), which axes crossed in the area of their intersection. On the front wall and rear wall of the housing block (5), around the through opening of the detection chamber (6), two M2 threaded holes, 5 mm deep, were drilled for mounting screws of the optical detection system elements (70), located in opposite corners of a square with of a side length of 8.5 mm, which geometrical centre lied at the axis of the detection chamber (6). In the housing block (5), in the vertical axis, four through holes with a diameter of 4 mm were drilled, ended with undercuts for the screw head and nut, located in the corners of the housing block (5), for screwing together its two parts when the cylinder (1) with the gasket (9) embedded. Then, the housing block was cut in a horizontal plane passing through the axis of the opening for the cylinder (1) and the opening for the detection chamber (6). Then, a quartz rod with an outer diameter of 10 mm and a length of 94 mm was placed in the hole for the cylinder (1) covered form the bottom and form the top with the fragments of the gasket (9) of the dimensions 10×94 mm, and then the elements of the housing block (5) were set together and secured with the mounting screws passing through the dedicated through holes. Then, in the housing block (5), perpendicularly to its base, in a vertical plane passing through the axis of the cylinder (1), channels (11,13,15,17) with an internal diameter of 1 mm were drilled, with four channels (11) drilled from the top of the housing block (5), and channels (13,15,17) from the bottom. The channels were drilled in the sequence 10A-12-10B-14-10C-16-10D of projections of their on the axis of the cylinder (1) with a spacing of 11 mm of these projections. The channels (11,13,15,17) were drilled in such a way that they did not drill through the quartz rod, but only reached its axis, drilling through the gasket (9). Then, a through hole with an internal diameter of 6 mm was drilled in the axis of the quartz rod, creating a quartz cylinder (1) with an internal diameter of 6 mm and a wall thickness of 2 mm, equipped with holes (10,12,14,16) in its walls, respectively opening the previously drilled channels (11,13,15,17), with an 11 mm spacing of their axes on the axis of the cylinder (1) and the sequence 10A-12-10B-14-10C-16-10D. Other manipulations were as in Example 10.

Example 12. An inverse cartridge (30) was manufactured for creatinine determination in the dialysate. For this purpose, the housing (31,32) of the cartridge (30) was taken, manufactures in Example 10, and then a syringe (20) with a piston (21) with a volume of 10 ml, made of polypropylene (PP), with a dispensing tip (22) of the LUER type located centrally in the axis of the syringe (20), with a valid medical device certificate, was placed in each of the LUER sockets (23). The syringes were filled according to the scheme ST-R1-R2-MIX:
(20A): 1800 μl aqueous solution of creatinine, 700 μM [CAS: 60-27-5]
(20B): 7100 μl aqueous solution of picric acid, 25 mM [CAS: 88-89-1]
(20C): 4100 μl aqueous solution of sodium hydroxide: 200 mM [CAS: 1310-73-2]
(20D): left empty with a piston in lower position (empty mixer)

Example 13. An inverse cartridge (30) was prepared for the determination of urea in the dialysate. For this purpose, the housing of the (30) manufactures in Example 10 was taken and prepared as in Example 12. Syringes were filled according to the scheme ST-R1-R2-MIX:
(20A): 1500 μl aqueous solution of urea, 16.0 mM [CAS: 54-13-6]
(20B): 3600 μl water/ethanol solution containing
4-(dimethylamine)benzaldehyde, 120 mM [CAS: 100-10-7]
78.5% v/v ethyl alcohol [CAS: 64-17-5], 72 mM hydrochloric acid [CAS: 7647-01-0]
(20C): 5300 μl aqueous solution of hydrochloric acid, 400 mM [CAS: 7647-01-0]
(20D): left empty with a piston in lower position (empty mixer)

Example 14. An inverse cartridge (30) was prepared for the determination of phosphates in the dialysate. For this purpose, the housing of the (30) manufactures in Example 10 was taken and prepared as in Example 12. Syringes were filled according to the scheme ST-R 1-R2-MIX:
(20A): 800 μl aqueous solution of sodium phosphate, 1000 μM [CAS:7601-54-9]
(20B) 3600 μl aqueous solution containing
ammonium ortomolibdenate, 20 mM [CAS: 236-031-3],
potassium antimony tartrate, 2.10 mM [CAS: 28300-74-5]
sulphuric(VI) acid, 800 mM [CAS: 76664-93-9]
(20C): 10700 μl aqueous solution of ascorbate acid, 120 mM [CAS: 50-81-7]
(20D): left empty with a piston in lower position (empty mixer)

Example 15. Dialysis progress was monitored by cyclic determination of creatinine concentration in the post-dialysis fluid of a patient with a terminal stage renal failure during a routine haemodialysis treatment (patient 16, dialysis 8) using a standard haemodialysis machine and the device according to the present invention. The device in the preferred embodiment described in Example 10 with an inverse cartridge (30) containing a reagent kit for the determination of creatinine in dialysate, described in Example 12.

The airlock (50) was connected to the waste channel (60) of the haemodialysis machine. The measurement procedure was preceded by washing the system. After switching on the haemodialysis machine, a portion of pure dialysis fluid (without the patient's uremic toxins) was collected in the accumulation reservoir (52) of the airlock (50), capable of retaining 60 ml of the solution. Then, a portion of this solution was taken through the hose (41) directly to the cylinder (1), filling the hose with a fresh portion of the dialysis fluid, which was then pumped through the cylinder (1) to the mixer (20D), and then discharged into the waste channel (61) alternately through the hoses (44,46), washing the walls of the entire system. The washing procedure was repeated 10 times. A two-point calibration was then performed using a portion of pure dialysate (zero point) and a standard form the reservoir (20A) (ST: 88 μl; R1: 141 μl; R2: 81 μl) according to the differential measurement procedure described below. After starting the dialysis process, periodically (every 5 min @0-100 min, every 10 min @100-180 min, every 15 min @100-180 min), a portion of 60 ml of dialysate was retained in the accumulation reservoir (52) of the airlock (50) for temporary storage. From this volume, 88 μl portions of dialysate were sampled through the hose (41) directly into the cylinder (1) and then pumped into the mixer (20D). Then, at every determination, portions of chemical reagents (R1: 141 μl, R2: 81 μl) were taken sequentially from the reservoir (20B,20C) to the cylinder (1) and pumped to the mixer (20D). The resulting reaction solution with a volume of 310 μl was mixed by pumping it between the mixer (20D) and the cylinder (1). Then, a 240 μl portion of the reaction solution was pumped into the detection chamber (6) in the cylinder (1). Twice the volume of the detection chamber (6) (113 μl) was used to wash the cylinder (1) in the area of the detection chamber (6) and completely fill it with the analysed reaction solution. 30 seconds after filling the detection chamber (6), the absorbance of the reaction solution was measured using the 525 nm diode of the light source (71) and the green channel of the detector (74). After another 60 seconds, the absorbance measurement of the reaction solution was repeated in the same detection system. The analytical signal was the difference between the first and second measurement, and the obtained value was converted to concentration based on the calibration curve and plotted in the function of time. After completion of the determination, the post-reaction solution was pumped out of the cylinder (1) through the channel (15), hose (46) and main reservoir (51) of the airlock (50) to the waste channel (61). Then, the rest of the post-reaction solution from the mixer (20D) was pumped out through the hole (16), the channel (17), the hose (44) and the main reservoir (51) of the airlock (50) to the waste channel (61). In case the analyte concentration readings were in line with the analytical predictions, the accumulation reservoir (52) was emptied and another 60 ml portion of dialysate was collected after a given time. In case the readings of the analyte concentration indicated a deviation from the expectations in relation to a given measurement of creatinine concentration, the analysis of a given portion of the retained dialysate was repeated—after the reading was confirmed, this point was marked on the time axis (e.g. 180 min in FIG. 34), and when it was excluded it was skipped. In the event of an extreme deviation of the reading from the analytical predictions, the device was ready to activate the alarm system (80).

Monitored dialysis lasted 240 minutes. The graph of the dynamics of changes of the concentration of creatinine in the dialysate, obtained using the device according to the invention, corresponds to the decrease in the concentration of creatinine in the patient's blood, monitored by the classic method of hospital analysis (Hitachi-Roche Cobas 6000), which confirms the correctness of the current method of monitoring the progress of the dialysis process. The results obtained in Example 15 are shown in FIG. 34 (right graph).

In this way, 273 actual dialysate samples were tested according to the invention. The observed creatinine concentrations were correlated with the values obtained by classical recommended methods (Hitachi-Roche Cobas 6000): y=(0.95±0.01)x+(0.55±0.03); R2=0.94; n=273. The obtained results are presented in the summary chart in FIG. 34 (left chart).

Example 16. Dialysis progress was monitored by cyclic determination of urea concentration in the post-dialysis fluid of a patient with termination-stage renal failure during a routine haemodialysis treatment (patient 1, dialysis 8) using a standard haemodialysis machine and the device according to the present invention. A device in the preferred embodiment described in Example 10 and an inverse cartridge (30) containing a reagent kit for the determination of urea in dialysate, described in Example 13.

Dialysis monitoring was performed as in Example 15, except that different volumes of standard/sample and reagents were used (ST: 71 μl; R1: 71 μl; R2: 106 μl) resulting in a reaction solution of a volume of 248 μl. During dialysis, deviations from the analytical predictions were observed, which resulted in a one-time measurement repetition (e.g. 120 min in FIG. 35).

Monitored dialysis lasted 240 minutes. The graph of the dynamics of changes in the concentration of urea in the dialysate, obtained using the device according to the invention, corresponds to the decrease in the concentration of urea in the patient's blood, monitored by the classic method of hospital analysis (Hitachi-Roche Cobas 6000), which confirms the correctness of the current method of monitoring the progress of the dialysis process. The results obtained in Example 16 are shown in FIG. 35 (right graph).

Thus, according to the invention, 270 actual dialysate samples were tested. The observed urea concentrations were correlated with the values obtained by classical recommended methods (Hitachi-Roche Cobas 6000): y=(1.05±0.01)x+(4.39±0.51); R2=0.96; n=270. The obtained results are presented in the summary chart in FIG. 35 (left chart).

Example 17. Dialysis progress was monitored by cyclic determination of phosphate ions concentration in the post-dialysis fluid of a patient with termination-stage renal failure during a routine haemodialysis treatment (patient 20, dialysis 7) using a standard haemodialysis machine and the device according to the present invention. A device in the preferred embodiment described in Example 10 and an inverse cartridge (30) containing a reagent kit for the determination of phosphate ions in dialysate, described in Example 14.

Dialysis monitoring was performed as in Example 15, except that different volumes of standard/sample and reagents were taken (ST: 35 μl; R1: 71 μl; R2: 212 μl) resulting in a reaction solution of a volume of 318 μl. No deviations from the analytical predictions were observed during the dialysis.

Monitored dialysis lasted 240 minutes. The graph of the dynamics of changes in the concentration of phosphates in the dialysate, obtained using the device according to the invention, corresponds to the decrease in the concentration of phosphates in the patient's blood, monitored by the classic method of hospital analysis (Hitachi-Roche Cobas 6000), which confirms the correctness of the current method of monitoring the progress of the dialysis process. The results obtained in Example 17 are shown in FIG. 36 (right graph).

In this way, 242 actual dialysate samples were tested according to the invention. The observed phosphate concentrations were correlated with the values obtained using the classical recommended methods (Hitachi-Roche Cobas 6000): y=(0.97±0.02)+(0.09±0.02); R2=0.92; n=242. The obtained results are presented in the summary chart in FIG. 36 (left chart).

Example 18. A device for the automated determination of an analyte in the liquid phase according to the invention was manufactures, in the preferred variant III, with a horizontally oriented cylinder (1) embedded in the device, the model and functional diagram of which are shown in FIG. 37, FIG. 38 and FIG. 39, and its functional elements are shown in FIG. 41, FIG. 42, FIG. 43, FIG. 48, FIG. 49, FIG. 33 and FIG. 56.

The housing block (5) with a cuboid shape, 94 mm long, 28 mm wide and 27 mm high, was made of peek using classical machining techniques. In the housing block (5), along its longitudinal axis, perpendicularly to its side walls, a through hole with an internal diameter of 6 mm was drilled, constituting a cylinder (1), perpendicular to the side walls of the housing block (5). Channels (11,13,15,17) with an internal diameter of 1 mm were also drilled, ending with holes (10,12,14,16) in the wall of cylinder (1) with a spacing of 11 mm on the axis of the cylinder (1) and sequence 10A-12-10B-14-10C-16-10D. Channels (11,13,15,17) were drilled in the housing block (5) perpendicularly to its surface, in a vertical plane passing through the axis of the cylinder (1), with the channels (11) drilled from the top of the housing block (5), and the channels (13,15,17) drilled from below. As pistons (2) a two-piece system was used, consisting of a PTFE piston rod with a diameter of 6.2 mm and a length of 30 mm, with a brass guide holder embedded, further connected through connectors (4), with the nuts on the lead screws (Tr 8×1) of the stepper motors (3). From the bottom of the housing block (5), four M3 threaded holes with a depth of 12 mm were drilled, receiving the mounting screws of the detection block (7), located in the corners of a square with a side length of 18 mm, with the geometrical centre lying on the axis of the channel (15). From the bottom of the housing block (5), four M4 threaded through holes were also drilled, for mounting on the device's supporting plate, located in the corners of a 53×20 mm rectangle, with the geometrical centre lying at the axis of the channel (15). From the top of the housing block (5), at the exit of each of the four holes (11), ports (28) with a diameter of 6.2 mm and a depth of 17.2 mm were drilled, with stepped undercuts (29) of a diameter of 10.25 mm and a depth of 5 mm, receiving the gaskets (27) in the shape of an O-RING with a diameter D/d of 6/2 mm. From the top of the housing block (5), between the ports (28A,28B) and (28C,28D), M4 threaded holes with a depth of 8 mm were drilled, receiving the pillars (38) positioning the cartridge (30) in relations to the housing block (5). From the top of the housing block (5), on its transverse axis, at a spacing of 21.8 mm, M3 threaded holes with a depth of 5.3 mm were drilled, located at the bottom of square depressions with a depth of 3.2 mm and a side length of 8 mm, centred in relation to the axis of the M3 holes, receiving the screws stabilising the position of the lid (26) in the vertical axis. From the bottom of the housing block (5), at the outlet of the channels (13,17), in the 3 mm thick protrusions, R1/8 threaded holes with a depth of 7 mm were drilled, accepting FESTO/SMC quick connect fittings. The housing block (5) in this variant is shown in FIG. 39, FIG. 41 and FIG. 43.

The compression lid (26) 94 mm long and 28 mm wide was made of PEEK using machining techniques. In the lid (26) through holes with an internal diameter of 6.2 mm and a height of 12.7 mm were milled, the wall thickness of the lid (26) was 2.5 mm, and the through holes protruded beyond the outline of the lid's plane at 6 mm from the top and 4.2 mm from the bottom. In the lid (26), on its transverse axis, at a spacing of 21.8 mm, through holes with a diameter of 3.5 mm were drilled, located in a square depression with a side length of 7 mm, protruding from the bottom beyond the plane of the lid (26) by 2.5 mm, so that the heads of the stabilising screws did not extend beyond the plane of the lid (26). The lid (26) in this variant is shown in FIG. 39, FIG. 41 and FIG. 43.

The detection block (7) with a length of 28 mm, a width of 28 mm and a height of 30 mm was made of PEEK using lossy machining techniques. The detection block (7) had a single horizontal three-compartment circular through opening, perpendicular to the side walls of the detection block (7), where the middle compartment was the detection chamber (6) with an internal diameter of 4 mm and a length of 5 mm, and the side compartments with a diameter of 8 mm and length of 11.5 mm served technical functions. The detection chamber (6) was closed with transparent windows (8) 1 mm thick and 8 mm in diameter, made of PMMA, placed in the side compartments, pressed against the detection chamber (6) with PEEK sleeves with an outer diameter of 8 mm and an inner diameter of 4 mm. In the detection block (5) a vertical channel (15,18) with an internal diameter of 1 mm was drilled along its vertical axis, passing through the centre of the detection chamber (6). In the detection block (7), four through-holes for mounting screws to the housing block (5) were drilled, located in the corners of a square with a side length of 18 mm, with the geometrical centre lying at the axis of the channels (15,18). The diameter of the mounting holes was 3.2 mm in the upper 17 mm long fragment, and 6 mm on the lower 13 mm long fragment, which task was to receive the heads of the assembly screws. From the bottom of the housing block (5), at the outlet of the channel (18), threaded hole R1/8 with a depth of 7 mm were drilled, accepting the FESTO/SMC quick connect fitting. On the side walls of the detection block (7), around the outlet of the opening containing the detection chamber (6), two M2 threaded holes, 5 mm deep, were drilled for the mounting screws of the optical detection system elements (70), located in opposite corners of the square with a side of 8.5 mm, with the geometrical centre lying at the axis of the detection chamber (6). The detection block (7) in this variant is shown in FIG. 39, FIG. 41 and FIG. 43.

The optical detection system (70) was combined with a light source (71) in the form of an integrated SMD LED diode, LUMIXTAR WL-1.5P5054EP120C3b1-RGV, and one universal detector (74) in the form of a CCD matrix with an RGB filter, HAMAMATSU 513683, with 40 zones, four channels, operating in RGB channels and a correction channel. The light source (71) was capable of emitting monochromatic light of three wavelengths: 525 nm (for the determination of creatinine), 415 nm (for the determination of urea) and 625 nm (for the determination of phosphates). The emitter (71) was placed on a 32×12 mm mounting plate with through-holes for M2 mounting screws, compatible with the mounting holes on the side walls of the detection block (7). The emitter (71) was mounted on the detection block (7) and centred on the axis of the detection chamber (6). The detector (74) was placed on a 32×12 mm mounting plate with through-holes for M2 mounting screws, compatible with the mounting holes on the side walls of the detection block (7). The mounting boards acted also as printed circuit boards equipped with connection paths for the SMD LED and the CCD matrix to the JST connector embedded on each board. The detector (74) was mounted on the detection block (7), on its other side in relation to the light source (71), on the axis of its optical path (72), and centred at the axis of the detection chamber (6). The optical detection system (70) in this variant is shown in FIG. 48 and FIG. 49.

The cartridge (30), 136 mm long, 41 mm wide and 95 mm high, with a cuboidal body of dimensions of 100×41×95 mm, with a wall thickness of 2 mm, equipped with side protrusions forming sockets (34), was made of ABS by injection technique. The sockets (34) on the two side walls of the cartridge housing (30) receiving the forks (35) of the lift (36) of the device were 4 mm wide and 27 mm high, and had a form of through undercuts on the inner planes of the side protrusions. The cartridge (30) consisted of a housing (31) and a lid (32), which were joined at the middle of the cartridge (30) by a one-time snap lock (33). The housing (31) (lower part) had LUER sockets (23) on the lower inner surface, receiving the LUER dispensing tips (22) of four reservoirs (20A,20B,20C,20D) in the form of syringes with a capacity of 10 ml each. The sockets (23) were connected by channels (24) of an internal diameter of 1 mm with through pins (25) extending beyond the lower outer surface of the cartridge (30), compatible with the ports (28) at the outlet of the channels (11) in the outer surface of the housing block (5) of the cylinder (1). The through pins (25) had an inner diameter of 1 mm, an outer diameter of 6 mm and a height of 16 mm. The outlet of each of the through pins (25A,25B,25C,25D) was secured from the bottom with a tear-off plate, protecting the content of the cartridge (30) against contamination. The cartridge (30) was equipped with an electronic circuit with non-volatile RFID NTAG213 memory with an 8×18 mm antenna, on an 11×21 mm adhesive backing, which was placed in a dedicated internal niche in the lower part of the housing (31). The cartridge (30) in this variant is shown in FIG. 41 i FIG. 42.

The housing block (5) and the detection block (7) were assembled by screwing them together with M3 screws with a length of 26 mm, using a 1 mm thick silicone gasket between the blocks (5,7), laser cut, equipped with holes coaxial with the channel (15) and with the holes for the mounting screws. The assembled blocks (5,7) were mounted on the supporting plate of the device with M4 screws 17 mm long, fixed in the through holes of the housing block (5). From the bottom, in the threaded holes at the outlet of the channels (13,17,18), FESTO/SMC quick connect fittings were placed, and the hoses (41,44,46) connecting the device with the airlock (50) were embedded in the fittings. Positioning pillars (38) with a diameter of 4.85 mm and a length of 25.6 mm, equipped with an M4 thread with a length of 6.20 mm, were mounted on the housing block (5). O-RING gaskets with a diameter of D/d 6/2 mm were placed in the ports (28). A lid (26) was put on the housing block and fixed with 8 mm M3 screws. Pistons (2) were inserted into the cylinder (1) in the housing block (5). Driving units with stepper motors (3) were mounted on the carrier plate and connected to the mini pistons (2) by means of connectors (4). An elevator (36) was assembled with forks (35) on vertical guiding pillars and connected to a drive equipped with a dedicated stepper motor. The whole setup was placed in a dedicated housing. The electronic main controller (88), equipped with antennas for remote communication, in a compact form and DIN standard housing was placed in a separate compartment in the back of the device. The electronic main controller (88) was connected with dedicated wires to the drive system of the pistons (2), the drive system of the lift (36), the RFID antenna, the optical detection system (70) and the airlock (50). A cartridge (30) filled with a set of reagents dedicated to determination of creatinine (Example 20) was taken, the protective tear-off plates were removed from its through pins (25A,25B,25C,25D) and placed on the fork (35) of the lift (36). The device prepared in this way was ready to monitor the concentration of creatinine in the dialysate.

Example 19. A device for automated determination of an analyte in the liquid phase according to the invention was manufactures, in the preferred embodiment, with a horizontally oriented cylinder (1) mounted as in example 18, with the difference that a one-piece housing block (5) was made integrated with the detection block (7). The functional diagram of this variant is shown in FIG. 40, and its functional elements are shown in FIG. 44.

The housing block (5) with a cuboidal shape, 94 mm long, 28 mm wide and 57 mm high, was made of PEEK using lossy machining techniques. In the housing block (5), along its longitudinal axis, a through hole with an internal diameter of 6 mm was drilled, constituting a cylinder (1), and channels (11,13,15,17,18) with an internal diameter of 1 mm, ended with holes (10,12,14,16) in the wall of the cylinder (1) with the sequence 10A-12-10B-14-10C-16-10D and 11 mm spacing of their projections on the axis of the cylinder (1), while the channels (15,18) were coaxial. Below the cylinder (1), in the housing block (5), one transverse horizontal three-compartment circular through opening was drilled, perpendicular to the front wall of the housing block (5) and perpendicular to the axis of the cylinder (1), where the middle compartment was the detection chamber (6) with an internal diameter of 4 mm and the length of 5 mm long, while side compartments with a diameter of 8 mm and a length of 11.5 mm served technical functions. The detection chamber (6) was closed with transparent windows (8) 1 mm thick and 8 mm in diameter, made of PMMA, placed in the side compartments, pressed against the detection chamber (6) with PEEK muffs with an outer diameter of 8 mm and an inner diameter of 4 mm. The other manipulations were as in Example 18.

Example 20. An inverse cartridge (30) was manufactured for creatinine determination in the dialysate. For this purpose, the housing (31,32) of the cartridge (30) was taken, manufactures in Example 18, and then a syringe (20) with a piston (21) with a volume of 10 ml, made of polypropylene (PP), with a dispensing tip (22) of the LUER type located centrally in the axis of the syringe (20), with a valid medical device certificate, was placed in each of the LUER sockets (23). The syringes were filled according to the scheme ST-R1-R2-MIX:
(20A): 1800 μl aqueous solution of creatinine, 700 μM [CAS: 60-27-5]
(20B): 7100 μl aqueous solution of picric acid, 25 mM [CAS: 88-89-1]
(20C): 4100 μl aqueous solution of sodium hydroxide, 200 mM [CAS: 1310-73-2]
(20D): left empty with a piston in lower position (empty mixer)

Example 13. An inverse cartridge (30) was prepared for the determination of urea in the dialysate. For this purpose, the housing of the (30) manufactures in Example 18 was taken and prepared as in Example 20. Syringes were filled according to the scheme ST-R1-R2-MIX:
(20A): 1500 μl aqueous solution of urea, 16.0 mM [CAS: 54-13-6]
(20B): 3600 μl water/ethanol solution containing:
4-(dimethylamine)benzaldehyde, 120 mM [CAS: 100-10-7]
78.5% v/v ethyl alcohol [CAS: 64-17-5], 72 mM hydrochloric acid [CAS: 7647-01-0]
(20C): 5300 μl aqueous solution of hydrochloric acid, 400 mM [CAS: 7647-01-0]
(20D): left empty with a piston in lower position (empty mixer)

Example 14. An inverse cartridge (30) was prepared for the determination of phosphates in the dialysate. For this purpose, the housing of the (30) manufactures in Example 18 was taken and prepared as in Example 20. Syringes were filled according to the scheme ST-R1-R2-MIX:
(20A): 800 μl aqueous solution of sodium phosphate, 1000 μM [CAS:7601-54-9]
(20B) 3600 μl aqueous solution containing:
ammonium ortomolibdenate, 20 mM [CAS: 236-031-3],
potassium antimony tartrate, 2.10 mM [CAS: 28300-74-5]
sulphuric(VI) acid, 800 mM [CAS: 76664-93-9]
(20C): 10700 μl aqueous solution of ascorbate acid, 120 mM [CAS: 50-81-7]
(20D): left empty with a piston in lower position (empty mixer)

Example 23. Dialysis progress was monitored by cyclic determination of creatinine concentration in the post-dialysis fluid of a patient with a terminal stage renal failure during a routine haemodialysis treatment (patient 23, dialysis 10) using a standard haemodialysis machine and the device according to the present invention. The device in the preferred embodiment described in Example 10 with an inverse cartridge (30) containing a reagent kit for the determination of creatinine in dialysate, described in Example 20.

The airlock (50) was connected to the waste channel (60) of the haemodialysis machine. The measurement procedure was preceded by washing the system. After switching on the haemodialysis machine, a portion of pure dialysis fluid (without the patient's uremic toxins) was collected in the accumulation reservoir (52) of the airlock (50), capable of retaining 60 ml of the solution. Then, a portion of this solution was taken through the hose (41) directly to the cylinder (1), filling the hose with a fresh portion of the dialysis fluid, which was then pumped through the cylinder (1) to the mixer (20D), and then discharged into the waste channel (61) through the detection chamber (6) and the hoses (44,46), washing the walls of the entire system. The washing procedure was repeated 10 times. A two-point calibration was then performed using a portion of pure dialysate (zero point) and a standard form the reservoir (20A) (ST: 88 μl; R1: 141 μl; R2: 81 μl) according to the differential measurement procedure described below. After starting the dialysis process, periodically (every 5 min @0-100 min, every 10 min @100-180 min, every 15 min @100-180 min), a portion of 60 ml of dialysate was retained in the accumulation reservoir (52) of the airlock (50) for temporary storage. From this volume, 88 μl portions of dialysate were sampled through the hose (41) directly into the cylinder (1) and then pumped into the mixer (20D). Then, at every determination, portions of chemical reagents (R1: 141 μl, R2: 81 μl) were taken sequentially from the reservoir (20B,20C) to the cylinder (1) and pumped to the mixer (20D). The resulting reaction solution with a volume of 310 μl was mixed by pumping it between the mixer (20D) and the cylinder (1). Then, a 240 μl portion of the reaction solution was pumped into the detection chamber (6) through the hole (14) and the channel (15). Four times the volume of the detection chamber (6) (63 μl) was used to wash the cylinder (1) in the area of the detection chamber (6) and completely fill it with the analysed reaction solution. 30 seconds after filling the detection chamber (6), the absorbance of the reaction solution was measured using the 525 nm diode of the light source (71) and the green channel of the detector (74). After another 60 seconds, the absorbance measurement of the reaction solution was repeated in the same detection system. The analytical signal was the difference between the first and second measurement, and the obtained value was converted to concentration based on the calibration curve and plotted in the function of time. After completion of the determination, the post-reaction solution was pumped out of the cylinder (1) through the channel (15,18), hose (46) and main reservoir (51) of the airlock (50) to the waste channel (61). Then, the rest of the post-reaction solution from the mixer (20D) was pumped out through the hole (16), the channel (17), the hose (44) and the main reservoir (51) of the airlock (50) to the waste channel (61). In case the analyte concentration readings were in line with the analytical predictions, the accumulation reservoir (52) was emptied and another 60 ml portion of dialysate was collected after a given time. In case the readings of the analyte concentration indicated a deviation from the expectations in relation to a given measurement of creatinine concentration, the analysis of a given portion of the retained dialysate was repeated—after the reading was confirmed, this point was marked on the time axis (e.g. 40 min, 55 min, 110 min, 120 min, 210 min in FIG. 52), and when it was excluded it was skipped. In the event of an extreme deviation of the reading from the analytical predictions, the device was ready to activate the alarm system (80).

Monitored dialysis lasted 240 minutes. The graph of the dynamics of changes of the concentration of creatinine in the dialysate, obtained using the device according to the invention, corresponds to the decrease in the concentration of creatinine in the patient's blood, monitored by the classic method of hospital analysis (Hitachi-Roche Cobas 6000), which confirms the correctness of the current method of monitoring the progress of the dialysis process. The results obtained in Example 23 are shown in FIG. 52 (right graph).

In this way, 953 actual dialysate samples were tested according to the invention. The observed creatinine concentrations were correlated with the values obtained by classical recommended methods (Hitachi-Roche Cobas 6000): y=(0,85±0,01)x+(0,45±0,02); R²=0,93; n=953. The obtained results are presented in the summary chart in FIG. 52 (left chart).

Example 24. Dialysis progress was monitored by cyclic determination of urea concentration in the post-dialysis fluid of a patient with termination-stage renal failure during a routine haemodialysis treatment (patient 8, dialysis 13) using a standard haemodialysis machine and the device according to the present invention. A device in the preferred embodiment described in Example 18 and an inverse cartridge (30) containing a reagent kit for the determination of urea in dialysate, described in Example 21.

Dialysis monitoring was performed as in Example 23, except that different volumes of standard/sample and reagents were used (ST: 71 μl; R1: 71 μl; R2: 106 μl) resulting in a reaction solution of a volume of 248 μl. During dialysis, deviations from the analytical predictions were observed, which resulted in a one-time measurement repetition (e.g. 15 min, 40 min, 80 min, 160 min, 210 min in FIG. 53).

Monitored dialysis lasted 240 minutes. The graph of the dynamics of changes in the concentration of urea in the dialysate, obtained using the device according to the invention, corresponds to the decrease in the concentration of urea in the patient's blood, monitored by the classic method of hospital analysis (Hitachi-Roche Cobas 6000), which confirms the correctness of the current method of monitoring the progress of the dialysis process. The results obtained in Example 24 are shown in FIG. 53 (right graph).

Thus, according to the invention, 1115 actual dialysate samples were tested. The observed urea concentrations were correlated with the values obtained by classical recommended methods (Hitachi-Roche Cobas 6000): y=(0,92±0,01)x−(2,47±0,31); R²=0,94; n=1115. The obtained results are presented in the summary chart in FIG. 53 (left chart).

Example 25. Dialysis progress was monitored by cyclic determination of phosphate ions concentration in the post-dialysis fluid of a patient with termination-stage renal failure during a routine haemodialysis treatment (patient 27, dialysis 16) using a standard haemodialysis machine and the device according to the present invention. A device in the preferred embodiment described in Example 18 and an inverse cartridge (30) containing a reagent kit for the determination of phosphate ions in dialysate, described in Example 22.

Dialysis monitoring was performed as in Example 15, except that different volumes of standard/sample and reagents were taken (ST: 35 μl; R1: 71 μl; R2: 212 μl) resulting in a reaction solution of a volume of 318 μl. During dialysis, deviations from the analytical predictions were observed, which resulted in a one-time measurement repetition (e.g. 15 min, 60 min, 80 min, 120 min, 210 min in FIG. 54).

Monitored dialysis lasted 240 minutes. The graph of the dynamics of changes in the concentration of phosphates in the dialysate, obtained using the device according to the invention, corresponds to the decrease in the concentration of phosphates in the patient's blood, monitored by the classic method of hospital analysis (Hitachi-Roche Cobas 6000), which confirms the correctness of the current method of monitoring the progress of the dialysis process. The results obtained in Example 25 are shown in FIG. 54 (right graph).

In this way, 838 actual dialysate samples were tested according to the invention. The observed phosphate concentrations were correlated with the values obtained using the classical recommended methods (Hitachi-Roche Cobas 6000): y=(1.07±0.01)x−(0.04±0.01); R²=0,90; n=838. The obtained results are presented in the summary chart in FIG. 54 (left chart).

### LIST OF DESIGNATORS OF FUNCTIONAL ELEMENTS OF THE DEVICE

1—cylinder
2—pistons of cylinder (1)
3—stepper motors with lead screws and positioning nuts to connect connectors (4)
4—connectors linking stepper motors (3) with pistons (2) of cylinders (1)
5—housing block
6—detection chamber
7—detection block (for variant III)
8—transparent windows sealing detection chamber (6) (for variant III)
9—gasket between the cylinder (1) and housing block (5) (for variant II)
10—hole in the wall of cylinder (1) towards reservoirs (20) in cartridge (30)
   A—hole towards reservoir (20A/95A) for the standard solution (ST)
   B—hole towards reservoir (20B/95B) for the first reagent (R1)
   C—hole towards reservoir (20C/95C) for the second reagent (R2)
   D—hole towards reservoir (20D) for mixing (MIX) or (95D) for matrix solution (MATRIX)
11—channel in housing block (5) as the extension of hole (10) in the wall of cylinder (1)
12—hole in the wall of cylinder (1) towards sample source (60,62,50)
13—channel in housing block (5) as the extension of hole (12) in the wall of cylinder (1)
14—hole in the wall of cylinder (1) towards detection chamber (6) or waste channel (61)
15—channel in housing block (5) as the extension of hole (14) in the wall of cylinder (1)
16—hole in the wall of cylinder (1) towards waste channel (sewage) (61)
17—channel in housing block (5) as the extension of hole (16) in the wall of cylinder (1)
18—channel in detection block (7) linking detection chamber (6) with waste channel (61) (for variant III)
19—gasket between housing block (5) and detection block (7) (for variant III)
20—reservoir in cartridge (30)—preferably: a medical syringe (for variants II and III)
   A—reservoir for the standard solution (ST)
   B—reservoir for the first reactant (R1)
   C—reservoir for the second reactant (R2)
   D—reservoir for mixing the reactants, i.e. mixer (MIX)
21—piston of reservoir (20)—preferably: a medical syringe piston
22—dispensing tip of reservoir (20)—preferably: LUER tip of a medical syringe
23—socket in the bottom part of the housing (31) of cartridge (30)—preferably: LUER or LUER LOCK
24—channel in the bottom part of the housing (31) linking socket (23) with pin (25)
25—through pin at the outlet of channel (24), compatible with port (28) in housing block (5)
26—lid compressing the gasket (27) when docking cartridge (30)
27—O-RING gasket side sealing for connecting pin (25) with port (28)
28—port in housing block (5) at the outlet of channel (11) receiving pin (25) of cartridge (30)
29—stepped undercut in port (28) receiving gasket (27)—preferably O-RING
30—replaceable inverse cartridge with reservoirs (20) (for variants II and III)
31—housing of cartridge (30) or bottom part of the housing of cartridge (30)
32—cap of cartridge (30) or upper part of the housing of cartridge (30)
33—lock connecting housing (31) and cover (32) of cartridge (30)
34—sockets in housing (31,32) receiving forks (35) of elevator (36) docking cartridge (30)
35—forks of lift (36) docking cartridge (30) to ports (28) at housing block (5) of cylinder (1)
36—lift docking the cartridge (30) to ports (28) at housing block (5) of cylinder (1)
37—holes receiving positioning pillars (37) of housing block (5)
38—pillars positioning cartridge (30) in relation to housing block (5)
39—electronic circuit of cartridge (30)
40—quick connect fitting with a gasket at the outlet of channel (13)—preferably: FESTO/SMC
41—sampling hose linking cylinder (1) with sample source (60,62,50)
42—ending of the sampling hose
43—quick connect fitting with a gasket at the outlet of channel (17)—preferably: FESTO/SMC
44—waste hose linking cylinder (1) with waste channel (sewage) (61)
45—quick connect fitting with a gasket at the outlet of channel (15/18)—preferably: FESTO/SMC
46—waste hose linking detection chamber (6) with waste channel (sewage) (61) or waste hose (44)
47—gas hose for pressure equalisation in the hydraulic system (for variant I)
50—airlock
51—main reservoir of airlock (50)
52—accumulation reservoir of airlock (50)
53—overflow channel of airlock (50)
54—valve at waste channel of accumulation reservoir (52)
55—valve at waste channel of main reservoir (51)
56—pressure sensor of airlock (50)
57—pneumatic hose of pressure sensor (56)
58—temperature sensor of airlock (50)
60—pipe with sample stream
61—waste channel (sewage)
62—automatic sample changer
63—gas source for pressure equalisation inside cylinder (1) (for variant I)
70—optical detection system
71—light source of optical detection system (70)
72—optical path of light source (71)
73—optical path perpendicular to optical path (72) of light source (71)
74—first detector
75—second detector
80—alarm system
81—speaker of alarm system (80)
82—light source of alarm system (80)
83—means of remote communication of alarm system (80)
88—electronic main controller of the device, equipped with antennas for remote communication
90—replaceable normal cartridge with reservoirs (95) (for variant I)
91—frame of cartridge (90)
92—upper part of frame of cartridge (90)
93—guides receiving frame (91) of cartridge (90)
94—lift of cartridge (90)
95—reservoir in cartridge (90): A (ST), B (R1), C (R2), D (MATRIX)—preferably conical vials
96—closing the reservoirs (95)
97—sockets for cups (96) of reservoirs (95)
98—tips receiving the endings of hoses (99)
99—hoses linking reservoirs (95) with holes (10) of cylinder (1)

1. A device for the automated determination of an analyte in the liquid phase by conducting:
a specific chemical reaction and subsequent optical measurement of the concentration of its products, containing a hydraulic system equipped with a set of hoses for pumping liquid solutions and reactants, a system for sampling the tested solution, a reservoir for a standard solution, reservoirs for selected chemical reagents, and an optical detection system for the determination of the product of the specific reaction, where the reservoirs for liquids are placed in a replaceable cartridge, which device has a reaction space to mix the sampled portion of the tested solution or the reference solution or the standard solution with selected chemical reagents, as well as a space for optical detection of the products of the specific reaction, and the device is electronically controlled by the main controller, equipped with means for communication and information transfer with external electronic devices, characterised in that it has a reaction-detection system,
where the reaction space takes the form of a cylinder (1) with pistons (2) moved by stepper motors (3), utilised to determine the selected analyte by conducting a specific chemical reaction and subsequent determination of its product using spectroscopic techniques,
equipped with a detection space (6) for quantitative determination of the product of the specific reaction in the post-reaction mixture,
equipped with an automated hydraulic system, allowing for precise sampling of the tested solution from the sample source (50,60,62), quantitative dosing of the tested solution and other reagents into the reaction space (1) and the detection space (6), as well as efficient gravitational pumping of the used liquid substances to the waste channel (61), whereby the flow of liquid in the hydraulic system is carried out pneumatically by changing the relative position of the pistons (2),
and equipped with a replaceable cartridge (30/90) with reservoirs (20/95) to store reagents and standards, an optical detection system (70), an airlock (50) protecting the sample source against microbial contamination, and an alarm system (80) for verbalising messages regarding the determination process, equipped with a speaker (81), a light source (82) and means for remote communication (83).

2. The device according to claim 1, characterised in that the cylinder (1), with two pistons (2) moved by means of stepper motors (3) connected to them via the connectors (4), constitutes the reaction space in the housing block (5), which cylinder (1) has holes (10, 12,14,16) connected with channels (11,13,15,17) in the housing block (5) respectively, wherein these holes and channels have in pairs (10-11, 12-13, 14-15, 16-17) the same diameter, and each of the at least four openings (10) and corresponding channels (11) is detachably connected to one reservoir (20) in the cartridge (30) via ports (28) with stepped undercuts (29) equipped with side sealing gaskets (27) and a pressing lid (26), which detachably receive through pins (25) of the cartridge (30), connected by channels (24) to the sockets (23) detachably receiving the dispensing tips (22) of the reservoirs (20), where the hole (12) and the channel (13) equipped with a quick connect fitting (40) are detachably connected by a sampling hose (41) to the source of the sample, i.e. to the automatic sampling system (62), the pipe (60) with the sample stream or the airlock (50) on the pipe (60), while the hole (16) and the channel (17) equipped with a quick connect fitting (43) are detachably connected by a waste hose (44) to the waste channel (61), while the hole (14) and the channel (15) are connected to the detection chamber (6) in the form of a transverse through opening in the detection block (7), sealed from the outside with transparent windows (8) cooperating with the elements of the optical detection system (70), while the detection chamber (6) through the channel (18) equipped with the quick connect fitting (45) is detachably connected by a waste hose (46) to the hose (44) or to the waste channel (61).

3. The device according to claim 2, characterised in that in the housing block (5), taking the form of a cuboid 85-105 mm long, 25-40 mm wide and 25-80 mm high, preferably with dimensions of 94×28×27 mm in the version with a separate detection block (7) or with dimensions of 94×28×57 mm in the version with an integrated detection block (7), the cylinder (1) is a through horizontal cavity, preferably with a circular cross-section, with an internal diameter in the range of 3-8 mm, preferably 4-7 mm, most preferably 6 mm, and a length in the range of 85-105 mm, preferably 94 mm, while its pistons (2), with a compatible outer diameter in the range of 3.2-8.2 mm, preferably 4.2-7.2 mm, most preferably 6.1 mm, tightly placed inside the cylinder (1), have piston rods made of a chemically inert, rigid plastic material such as polyethylene terephthalate (PTFE), polyetheretherketone (PEEK), poly(acrylonitrile-co-butadiene-co-styrene) (ABS), polyamide (PA) or polypropylene (PP), optionally in configuration with a gasket, preferably flat, and the guide holders of the piston rods made of metal such as brass, aluminium or steel, while the detection block (7) is in the form of a cuboid 30-50 mm long, 25-40 mm wide and 25-40 mm high, preferably with dimensions of 28×28×30 mm, or the detection block (7) is in the form of a cylinder with a diameter of 20-50 mm and a height of 25-40 mm, preferably 28 mm in diameter and 30 mm high, wherein the detection block (7) has at least one through opening constituting a detection chamber (6), preferably with a circular cross-section, with an internal diameter in the range of 3-10 mm, preferably 4-6 mm, most preferably 4 mm, sealed with transparent windows (8) made of a chemically inert material transparent in the range of determination of the product of specific reaction, preferably made of acrylic glass (PMMA), polycarbonate (PC) or polystyrene (PS), as well as a through channel (15) connecting the cylinder (1) with the detection chamber (6) and a through channel (18) connecting the detection chamber (6) with the quick connect fitting (45), wherein the channels (15,18) have a diameter in the range of 0.8-2 mm, preferably 1 mm, and preferably are perpendicular to the axis of the detection chamber (6), wherein the housing block (5) and the detection block (7) are made of a chemically inert, rigid material, preferably polyetheretherketone (PEEK), acrylic glass (PMMA), polyamide (PA) poly(acrylonitrile-co-butadiene-co-styrene) (ABS), aluminium or stainless steel, and preferably detection block (7) is rigidly and detachably connected to the housing block (5), and the detection chamber (6) is formed by two perpendicular, through openings, preferably perpendicular to the channels (15,18).

4. The device according to claim 1, characterised in that the cylinder (1), with two pistons (2) moved by means of stepper motors (3) connected to them via the connectors (4), is preferably horizontally oriented and tightly embedded inside the housing block (5) and tightly connected with it by its outer surface, is made of glass or quartz and equipped with at least one gasket (9), or is made of acrylic glass (PMMA), polystyrene (PS), polycarbonate (PC), poly(ethylene terephthalate) (PET) or polypropylene (PP), has holes (10, 12,14,16) connected with channels (11,13,15,17) in the housing block (5) respectively, wherein these holes and channels have in pairs (10-11, 12-13, 14-15, 16-17) the same diameter, and each of the at least four openings (10) and corresponding channels (11) is detachably connected to one reservoir (20) in the cartridge (30) via ports (28) with stepped undercuts (29) equipped with side sealing gaskets (27) and a pressing lid (26), which detachably receive through pins (25) of the cartridge (30), connected by channels (24) to the sockets (23) detachably receiving the dispensing tips (22) of the reservoirs (20), where the hole (12) and the channel (13) equipped with a quick connect fitting (40) are detachably connected by a sampling hose (41) to the source of the sample, i.e. to the automatic sampling system (62), the pipe (60) with the sample stream or the airlock (50) on the pipe (60), while the holes (14,16) and the channels (15,17) equipped with quick connect fittings (45,43) are detachably connected by a waste hose (46,44) to the waste channel (61), and furthermore, the housing block (5) has at least one a transverse through opening with a circular cross-section, constituting a detection chamber (6), revealing the transparent walls of the cylinder (1), allowing for the assembly of the elements of the optical detection system (70) on both its sides, while the detection chamber (6) and cylinder (1) are perpendicular to each other and their axes intersect, preferably directly above the outlet of the hole (14) and the channel (15).

5. The device according to claim 4, characterised in that in the housing block (5) in the form of a cuboid 85-105 mm long, 25-40 mm wide and 25-80 mm high, preferably with dimensions of 94×28×33 mm, there is a cylindrical through opening with an internal diameter in the range of 5-15 mm receiving cylinder (1), preferably 10 mm or 12 mm per cylinder (1) or cylinder (1) with a seal (9), respectively, and the cylinder (1) has a length in the range of 85-105 mm, preferably 94 mm, and an internal diameter in the range of 3-8 mm, preferably 4-7 mm, most preferably 6 mm, and an outer diameter in the range of 4-14 mm, preferably 10 mm, where the preferred wall thickness of the cylinder (1) is 2 mm, while its pistons (2) having a compatible outer diameter in the range of 3.0-8.2 mm, preferably 3.15-7.2 mm, most preferably 6.1 mm, tightly and firmly seated inside the cylinder (1), have piston rods made of a chemically inert, rigid plastic material such as polyethylene terephthalate (PTFE), polyetheretherketone (PEEK), poly(acrylonitrile-co-butadiene-co-styrene) (ABS), polyamide (PA) or polypropylene (PP), optionally in configuration with a gasket, preferably flat, and the guide holders of the piston rods made of metal such as brass, aluminium or steel, while the housing block (5) has at least one through opening constituting the detection chamber (6), preferably with a circular cross-section, with an internal diameter in the range of 3-10 mm, preferably 4-6 mm, most preferably 4 mm, where the through channel (15) connects the cylinder (1) with the quick coupler (45), wherein the channel (15) has a diameter in the range of 0.8-2 mm, preferably 1 mm, while the housing block (5) is made of a rigid chemically inert material, preferably polyetheretherketone (PEEK), acrylic (PMMA), polyamide (PA), poly(acrylonitrile-co-butadiene-co-styrene) (ABS), aluminium or stainless steel.

6. The device according to claim 2 or 4, characterised in that the inverse cartridge (30) has at least four reservoirs (20A,20B,20C,20D), preferably in the form of syringes with pistons (21), made of chemically inert materials, with a volume in the range of 5-12 ml, preferably 10 ml, with dispensing tips (22), preferably LUER, with outlets oriented downwards are embedded detachably in the sockets (23), preferably LUER or LUER LOCK, at the bottom of the housing (31) of the cartridge (30), wherein the cartridge (30) has a form of a container consisting of consists of at least a housing (31), a cover (32) and a lock (33), preferably a one-time lock, where the housing (31,32) of the cartridge (30) additionally has side sockets (34) for the forks (35) of the lift (36), made of one bent metal element fixed in four points on the lift (36), wherein the construction material of the cartridge (30) is thermoplastic, and additionally the cartridge (30) has an electronic system (39) equipped with a non-volatile memory (NFC RFID chip), wirelessly connected to the antenna of the electronic main controller (88) of the device when cartridge (30) docked in the device, which memory is recognised by the electronic main controller (88) to permit a single use of the cartridge (30).

7. The device according to claim 1, characterised in that the reaction space and the optical detection space is a cylinder (1) with transparent walls the range of determination of the product of the specific reaction, equipped with two opposing coaxial pistons (2) tightly sealing in on each side, moved by electronically controlled stepper motors (3), driving the pistons (2) in linear movement inside the cylinder (1), which is equipped with: a set of at least four hoses (99A,99B,99C,99D) embedded in the holes (10) in the wall of the cylinder (1), supplying liquid substances from at least four reservoirs (95A,95B,95C,95D) directly to the interior of the cylinder (1), including the tested solution from the sample source (50,60,62), as well as a hose (44) embedded in the hole (16) in the wall of the cylinder (1), removing liquid substances to the waste channel (61) directly from the interior of the cylinder (1), and at least one hose (47) embedded in the hole (14) in the wall of the cylinder (1), used to transfer gas and equalize the pressure inside the cylinder (1), while the fluid flow in the hydraulic system is carried out pneumatically by changing the relative mutual position of the pistons (2) generating gas pressure changes in a specific part of this system, forcing fluid movement to balance these changes, as well as at least one optical detection system (70), which components are placed around the cylinder (1) so that the optical path (72) connecting the light source (71) and the detector (74) passes through the interior of the cylinder (1).

8. The device according to claim 7, characterised in that the normal cartridge (90) has at least four reservoirs (95A,95B,95C,95D), preferably in the form of conical vials, with a volume of 10-50 ml, preferably 25 ml, 50-100 mm high, preferably 78 mm, with the reservoirs (95) having a closure (96) at the top, preferably in the form of a cap or cork, with a hole of a diameter of 4-20 mm, preferably 6-14 mm, preferably closed with septum, having a 1 mm hole for pressure equalisation, where the reservoirs (95) are rigidly placed with the opening upwards in the sockets (97) receiving the closure (96) from the bottom, where the internal diameter of the sockets (97) corresponds to the external diameter of the closures (96), while the sockets (97) are preferably equipped with rigid tips (98) stabilising the outlets of the hoses (99) connecting the reservoirs (95) with the holes (10) in the cylinder (1), wherein the cartridge (90) has a form of a container having a frame (91) and elements (92,97) positioning the reservoirs (95), where the frame (91) cooperates with the guides (93) and the lift (94) for docking the cartridge (90) in the device, preferably the cartridge (90) consists of two interconnecting parts, where the first includes frame (91), cylinder (1) with pistons (2), connectors (4), hoses (99), gas hose/hoses (47), sockets (97) and guides with a stabilising element accommodating the second part (92) containing the positioning elements of the reservoirs (95), wherein the parts (91,92) are detachably connected to each other in an unambiguous manner, ensuring the concentricity of the reservoirs (95) and the sockets (97), thanks to which placing the reservoirs (95) is carried out in a repetitive manner, whereby the hose (41) connecting the cylinder (1) with the sample source (50,60,62) is divided into two fragments, one of which (41A), tightly seated in the hole (12), is located in the cartridge (90) and terminated with a connector (41B), and the other (41D) starts with a connector (41C) compatible with the connector (41B) and continues in the body of the device connecting to the sample source (50,60,62), while the hose (44), connecting the cylinder (1) with the waste channel (61), is divided into two fragments, one of which (44A), tightly seated in the hole (16), is located in the cartridge (90) and ends with a connector (44B), and the second one (44D) starts with a connector (44C), compatible with the connector (44B), and continues in the body of the device connecting to the waste channel (61).

9. The device according to claim 2 or 4 or 7, characterised in that the optical detection system (70) consists of a light source (71), for example in the form of a diode, a fluorescent lamp or a light bulb, oriented with the front towards the interior of the detection space, and optionally one detector (74) or two detectors (74,75), for example in the form of a diode, photodiode, photoresistor, photomultiplier tube, CCD array or CMOS array, one of which (74), for photometric or turbidimetric detection, facing the interior of the detection space, is located on the axis of the optical path (72) of the light source (71) on the opposite side of the detection space, while the other detector (75), for fluorimetric or nephelometric detection, oriented with the front towards the interior of the detection space, is located on the axis of the optical path (73) crossing at 90° with the optical path (72) of the light source (71), wherein the light source (71) and detectors (74,75) can be guided to a desired location via optical fibres, wherein the light source (71) emitting light of adjustable wavelength, preferably equipped with a monochromator, or white light with a continuous spectrum, or monochromatic light in the range of absorption or excitation of the product of the specific reaction, or monochromatic light of several wavelengths in the range of absorption or excitation of the products of the specific reactions, while the detectors (74,75) are adapted to a specific analyte and a specific light source (71), and in particular the radiation of the light source (71) and the detectors (74,75) are adapted to the determination of creatinine, urea and phosphate ions, while monitoring the progress of the toxin removal process during the dialysis.

10. The device according to claim 2 or 4 or 7, characterised in that the sample source is a classic sampling system (62) in the form of an automatic sample changer, or the sample source is a pipe with sample stream (60), or the sample source is an airlock (50) through which the sample stream is passing through the pipe (60), preferably the sample is taken from the accumulation reservoir (52) of the airlock (50) or its waste channel, wherein the airlock (50) is an open system and preferably the walls of the sample stream pipe (60) are not in contact with the housing of the main reservoir (51) and during the monitoring of the progress of the dialysis process, the sample source is an airlock (50) mounted on the pipe (60) with the dialysate stream flowing directly from the dialyser.

11. A method of automated determination of an analyte in the liquid phase by conducting a specific chemical reaction and subsequent optical measurement of the concentration of its products, characterised in that it uses the device for automated determination of an analyte in the liquid phase with a reaction-detection unit equipped with a replaceable cartridge (30/90), in particular for monitoring the progress of the dialysis process, described in claims 1-10, selecting a specific chemical reaction matching to a specific analyte and the wavelength for determining the product of this specific reaction, after which the device is adapted to the selected determination by adjusting the optical detection system (70), and adjusting the content of the cartridge (30/90) filling its first reservoir (20A/95A) with a standard solution, and two subsequent reservoirs (20B/95B,20C/95C) with chemical reagents necessary to carry out the specific reaction, where the fourth reservoir (20D) in the inverse cartridge (30) acts as a mixer, and the fourth reservoir (95D) in the normal cartridge (90) is used to store the matrix solution, wherein and then the cartridge (30/90) is placed in the device, and then the solution to be determined is taken by sucking its portion into the cylinder (1) through the sampling hose (41) from the sample source (60,62,50) or the standard solution from the reservoir (20A) or the matrix solution from the sample source (60,62,50) or the reservoir (95D) and then portions of chemical reagents are sequentially sampled from the two reservoirs (20B/95B,20C/95C) of the cartridge (30/90), into the cylinder (1), after which the reaction solution is mixed, and then the reaction solution is transferred to the area of the optical detection system (70), ensuring the liquid level in this area allowing the optical path (72) of the light source (71) to pass through the solution to be determined, preferably the level completely covering the optical path (72), and after a certain time the concentration of the product of the specific reaction is optically determined using the optical detection system (70) by photometric, turbidimetric, fluorimetric, nephelometric measurement, or their combination, and then the post-reaction solution is pumped out to the waste channel (61), cylinder (1) is cleaned by washing it with a fresh portion of the tested solution drawn into the cylinder (1) through the sampling hose (41), which is then pumped out from the cylinder (1) into the waste channel (61), wherein fluid flow in the hydraulic system is generated pneumatically by changing the relative position of the pistons (2) in the cylinder (1), and in cases where it is necessary to move the reaction solution to the desired area of the cylinder (1), the pistons (2) are moved in the hydraulic and pneumatic neutral mode with the same speed, direction and sense inside the cylinder (1), wherein while monitoring the progress of dialysis, when the analyte concentration readings indicate that the toxins have reached the normative level, which would be observed for a healthy person, indicating the effective purification of the patient's blood, the alarm system (80) is automatically activated, informing about the possibility of termination of the dialysis, or when the toxin level behaves abnormally, the alarm system (80) is automatically activated, informing about the possible errors in the dialysis process, and in both cases, the alarm system (80) automatically activates a sound and a light signal on the device, and sends information to peripheral devices such as a display on the device or the operator's phone, and the measurement for the proper sample is preceded by calibration measurements using the standard solution and the matrix solution.

12. The method according to claim 11, characterised in that using the device in the variant described in claims 1,2,3,6,9,10 (so-called variant III), immediately after sequential uptake of the tested sample and reagents from the reservoirs (20B,20C) of the cartridge (30) into the cylinder (1), they are sequentially pumped into the mixer (20D), and after taking and pumping all the solutions to the fourth reservoir (20D), the obtained reaction solution is mixed by its pumping between the cylinder (1) and the reservoir (20D), where the volume of the tested sample equals 30-90 μl, the volume of the reagents used equals 50-250 μl, which gives a reaction mixture of a volume of 240-320 μl, and after mixing the reaction solution, its portion, preferably 240 μl, is pumped from the mixer (20D) to the cylinder (1), and then through the hole (14) and channel (15) to the detection chamber (6), where the optical measurement is carried out allowing for the quantitative determination of the analyte, and after the determination, the reaction solution is pumped out form the detection chamber (6) and the reservoir (20D) through the channel (18) and a channel (17), respectively, to the waste channel (61).

13. The method according to claim 11, characterised in that using the device in the variant described in claims 1,4,5,6,9,10 (so-called variant II), immediately after sequential uptake of the tested sample and reagents from the reservoirs (20B,20C) of the cartridge (30) into the cylinder (1), they are sequentially pumped into the mixer (20D), and after taking and pumping all the solutions to the fourth reservoir (20D), the obtained reaction solution is mixed by its pumping between the cylinder (1) and the reservoir (20D), where the volume of the tested sample equals 30-90 μl, the volume of the reagents used equals 50-250 μl, which gives a reaction mixture of a volume of 240-320 μl, and after mixing the reaction solution, its portion, preferably 240 μl, is pumped from the mixer (20D) to the cylinder (1), and then moved between the pistons (2) to the area of the detection chamber (6) in the cylinder (1), where the optical measurement is carried out allowing for the quantitative determination of the analyte, and after the determination, the reaction solution is pumped out form the detection chamber (6) and the reservoir (20D) through the channel (15) and a channel (17), respectively, to the waste channel (61).

14. The method according to claim 11, characterised in that using the device in the variant described in claims 1,7,8,9,10 (so-called variant I), after sequential uptake of the test sample and reagents from the reservoirs (95B,95C) of the cartridge (90) into the cylinder (1) while replenishing or reducing the gas content in the cylinder (1) between the pistons (2) using the hole (14) and the gas hose (47), the obtained reaction solution is mixed by passing gas taken from the hose (47) through its volume, wherein the volume of the reaction solution being preferably 428-1040 μl in the system with a horizontal cylinder (1) or 565-1040 μl in the system with a vertical cylinder (1), where the width of the optical path (72) equals 5 mm and the distance between the projections of the holes (10,12,14,16) on the cylinder (1) axis equals 5 mm, and after mixing the reaction solution, a portion of the reaction solution is moved between the pistons (2) to the detection area in the cylinder (1), where the analyte is determined, and after the determination, the reaction solution is pumped out from the cylinder (1) from in the detection area through the opening (16) and the hose (44) to the waste channel (61).

15. The method according to claim 11, characterised in that to track the progress of blood dialysis by examining the changes in the level of the toxins in the stream of the post-dialysis fluid flowing out form artificial kidney through its waste channel (60), it is used
a device having one optical detection system (70) containing a light source (71) emitting light of adjustable wavelength or white light of a continuous spectrum, and a universal detector (74), or
a device having at least 3 optical detection systems (70) containing various light sources (71) emitting monochromatic light of a wavelength of 500-550 nm, preferably 525 nm, 410-460 nm, preferably 415 nm, and 550-900 nm, preferably 625 nm, and various detectors (74) of a wavelength of 525 nm, 460 nm and 625 nm, respectively, and identical detectors (75) of a wavelength of 625 nm, or
a device having a optical detection system (70) comprising a combined light source (71) emitting monochromatic radiation of a wavelength of 500-550 nm, preferably 525 nm, 410-460 nm, preferably 415 nm, and 550-900 nm, preferably 625 nm, and a universal detector (74),
wherein, before monitoring the progress of dialysis, the analyte (toxin) for the determination is selected from: creatinine, urea and phosphate ions, and then a standard solution is placed in the reservoir (20A/95A), and chemical reagents to conduct the specific reaction are placed in the reservoirs (20B/95B,20C/95C), respectively:
when determining creatinine: standard creatinine aqueous solution [CAS 60-27-5], picric acid aqueous solution [CAS 88-89-1], and aqueous solution of NaOH[CAS 1310-73-2],
for the determination of urea: standard aqueous solution of urea [CAS 54-13-6], aqueous-ethanolic solution of 4-(dimethylamine)benzaldehyde [CAS 100-10-7] and hydrochloric acid [CAS 7647-01-0], and aqueous solution of HCl [CAS 7647-01-0],
and for the determination of phosphate ions: a standard aqueous solution of sodium phosphate [CAS:7601-54-9], an aqueous solution containing ammonium orthomolybdate [CAS 236-031-3], potassium antimonyl tartrate [CAS 28300-74-5], sulfuric acid [CAS 76664-93-9] and an aqueous solution of ascorbic acid [CAS: 50-81-7],
and then the cartridge (30/90) is mounted in the device, wherein the matrix solution is the pure dialysis fluid, which before starting the actual dialysis is sampled from the waste stream of the artificial kidney (60) through the airlock (50), after which a preliminary calibration measurement is carried out, and then the post-dialysis fluid is sampled from the waste stream of the artificial kidney (60) through the airlock (50), at given intervals, e.g. every 5-15 minutes, and the temporary concentration of the analyte in the dialysate stream (60) is determined, preferably washing the cylinder (1) between the sequential samplings and determinations of the analyte with a portion of the dialysate stream of the current composition, and preferably by carrying out calibration measurements between successive samplings and determinations of the analyte, using the standard solution from the reservoir (20A), simultaneously tracking on an external electronic device the decrease in the toxin content in the dialysate in the function of time.
